(19) United States
(12) Patent Application Publication
(10) Pub. No.: US 2025/0171506 A1
(43) Pub. Date: May 29, 2025

(54) CHIMERIC PAPILLOMAVIRUS L1 PROTEIN

(71) Applicant: SINOCELLTECH LTD., Beijing (CN)

(72) Inventors: Liangzhi Xie, Beijing (CN); Chunxia Luo, Beijing (CN); Wei Zhang, Beijing (CN); Xiaoyan Suo, Beijing (CN); Lin Pang, Beijing (CN); Ping Hu, Beijing (CN)

(21) Appl. No.: 18/904,623

(22) Filed: Oct. 2, 2024

Related U.S. Application Data
(63) Continuation of application No. 17/579,447, filed on Jan. 19, 2022, now Pat. No. 12,139,514.
Application No. 17/579,447 is a continuation of application No. PCT/CN2020/102603, filed on Jul. 17, 2020.

(30) Foreign Application Priority Data
Jul. 19, 2019 (CN) 201910656278.7

(51) Int. Cl. C07K 14/025; A61K 39/00; A61K 39/12; A61P 31/20; C12N 7/00

(52) U.S. Cl. CPC C07K 14/025; A61K 39/12; A61P 31/20; C12N 7/00; A61K 2039/5258; C12N 2710/20022; C12N 2710/20023; C12N 2710/20034

(57) ABSTRACT

The present invention relates to chimeric papilloma virus L1 proteins and polynucleotides encoding thereof, and also to HPV virus-like particles and the preparation methods thereof. Said chimeric papilloma virus L1 protein comprises an N-terminal fragment derived from L1 protein of the first papilloma virus type, said N-terminal fragment maintains the immunogenicity of the L1 protein of the corresponding type of HPV; and a C-terminal fragment derived from L1 protein of the second papilloma virus type, said L1 protein of the second papilloma virus type has a better expression level and a better solubility compared to the L1 proteins of other HPV types; wherein said chimeric papilloma virus L1 proteins have the immunogenicity of the L1 proteins of the corresponding HPV types. Said chimeric papilloma virus L proteins have better expression amount and solubility for mass production of vaccines.

### CROSS-REFERENCE SECTION

This application is a continuation of and claims priority to U.S. application Ser. No. 17/579,447 filed Jan. 19, 2022, which itself is a continuation of and claims priority to PCT Application No. PCT/CN2020/102603 filed Jul. 17, 2020, which itself claims priority to Chinese Patent Application No. 201910656278.7 filed Jul. 19, 2019. The contents from all the above are hereby incorporated in their entirety by reference.

### REFERENCE TO AN ELECTRONIC SEQUENCE LISTING

The contents of the electronic sequence listing (92568707 Sequence Listing_V2.xml; Size: 343 kilobytes; and Date of Creation: Oct. 1, 2024) is herein incorporated by reference in its entirety.

### FIELD OF THE INVENTION

The present invention relates to the papilloma virus (HPV) L1 protein and the polynucleotide encoding the same, and also to HPV virus-like particles and the preparation methods thereof.

### BACKGROUND OF THE INVENTION

Papilloma virus (PV) belongs to the Papillomaviridae Family and causes papillomas in humans, cattles, dogs, and rabbits. One of its member, human papilloma virus (HPV), is a non-enveloped DNA virus. The genome of this virus is a double-stranded closed circular DNA, about 7.2-8 kb in size, with 8 open reading frames, which can be divided into three regions according to their functions: (1) early region (E), about 4.5 kb, encoding E1, E2, E4-E7, a total of 6 non-structural proteins related to viral replication, transcription and transformation; (2) late region (L), about 2.5 kb, encoding the major capsid protein L1 and the minor capsid protein L2; (3) long regulatory region (LCR), which is located between the end of the L region and the beginning of the E region, is about 800-900 bp long and does not encode any protein, serving as DNA replication and expression regulatory elements.

The L1 proteins and the L2 proteins are synthesized late in the HPV infection cycle. The L1 protein is the major capsid protein and has a molecular weight of 55-60 kDa. The L2 protein is the minor capsid protein. 72 L1 protein-pentamers form the outer shell of the icosahedral HPV particle (45-55 nm in diameter), which encloses closed circular double-stranded DNA. The L2 protein is located on the inner side of the L1 protein (Structure of Small Virus-like Particles Assembled from the L1 Protein of Human Papillomavirus 16 Chen, X. S., R. L. Garcea, Mol.Cell.5 (3): 557-567, 2000).

The ORF of the L1 protein, the most conserved gene in the PV genome, can be used to identify new PV types. A new PV type is identified if its complete genome is cloned and its L1 ORF DNA sequence differs from the closest known PV type by more than 10%. Homologies with differences between 2% and 10% are defined as different subtypes, and differences of less than 2% are defined as different variants of the same subtype (E.-M. de Villiers et al./Virology 324 (2004) 17-27).

At late stages of HPV infection, newly synthesized L1 proteins in the cytoplasm are transported to the nucleus of terminally differentiated keratin where, together with L2 proteins, package the replicated HPV genomic DNA to form infectious viruses (Nelson, L. M, et al. 2002. Nuclear import strategies of high risk HPV16 L1 major capsid protein. J. Biol. Chem. 277:23958-23964). This suggests that nuclear import of the L1 protein plays a very important role in HPV infection and production. The ability of the virus to enter the nucleus is determined by the nuclear localization signal (NLS) at the C-terminus of the HPV L1 protein, the NLS is characterized by its abundance of basic amino acids (Garcia-Bustos, J., et al. 1991. Nuclear protein localization. Biochimica et Biophysica Acta 1071:83-101).

15 high-risk (HR) HPV types can lead to cancers of cervix, anus, penis, vagina, vulva and oropharynx, among which HPV-16 and HPV-18 are by far the most common causes of cancers, accounting for approximately 70% of cervical cancers, and the other HR-HPV types (Types 31, 33, 35, 39, 45, 51, 52, 56, 58, 59, 68, 73, and 82) cause the rest. HPV-16 accounts for approximately 95% of HPV-positive oropharyngeal cancers (OPCs). The persistent low-risk genotypes HPV-6 and HPV-11 cause most anogenital warts and respiratory papillomas, but are rarely associated with cancers (Human Papillomavirus in Cervical Cancer and Oropharyngeal Cancer: One Cause, Two Diseases Tara A. Bermanand John T. Schiller, PhD2 Cancer 2017; 123:2219-29).

The L1 protein can be recombinantly expressed by poxvirus, baculovirus, or yeast systems and then self-assembles to form virus-like particles (VLP) containing approximately 72 L1 proteins, similar to the virus capsid. VLP has no indication. VLP induces neutralizing antibodies in inoculated animals and protects experimental animals from subsequent attack by infectious viruses. Thus, VLP appears to be an excellent candidate for papilloma virus vaccines (Structure of Small Virus-like Particles Assembled from the L1 Protein of Human Papillomavirus 16 Chen, X. S., R. L. Garcea, Mol. Cell. 5 (3): 557-567, 2000).

Glaxo's CERVARIXR, a bivalent recombinant HPV vaccine, contains HPV Type 16 recombinant L1 protein and HPV Type 18 recombinant L1 protein. The L1 protein is obtained by expression of a recombinant baculovirus expression vector system in insect cells of the nocturnal moth (Trichoplusia ni). The L1 protein self-assembles into virus-like particles for the prevention of cervical cancer, Grade 2 or 3 cervical intraepithelial neoplasia and adenocarcinoma in situ caused by HPV Types 16 and 18, and Grade 1 cervical intraepithelial neoplasia (oncogenic) in women aged 9-25 years.

GARDASIL® is a human papilloma virus quadrivalent (Types 6, 11, 16 and 18) recombinant vaccine from Merck for the prevention of cervical cancer, genital warts (condyloma acuminata) and precancerous or proliferative abnormal lesions caused by HPV Types 6, 11, 16 and 18 in girls and women aged 9-26 years; and for the prevention of anal cancer, genital warts (condyloma acuminatum) and pre-cancerous or abnormal developmental lesions caused by HPV Types 6, 11, 16, and 18 in boys and men aged 9-26.

GARDASIL® 9 is a nine-valent recombinant human papilloma virus vaccine from Merck that contains virus-like particles of L1 proteins of HPV Types 6, 11, 16, 18, 31, 33, 45, 52 and 58, the L1 protein is produced by fermentation of Saccharomyces cerevisiae and self-assembles into VLP. It is used in girls and women aged 9-45 years for the prevention of cervical cancer, vulvar cancer, vaginal cancer and anal cancer caused by HPV Types 16, 18, 31, 33, 45, 52 and 58, genital warts (condyloma acuminata) caused by HPV Types 6 and 11, and precancerous or proliferative abnormalities caused by HPV Types 6, 11, 16, 18, 31, 33, 45, 52 and 58; and in boys and men aged 9-45 years for the prevention of anal cancer caused by HPV Types 16, 18, 31, 33, 45, 52 and 58, genital warts (condyloma acuminatum) caused by HPV Types 6 and 11 and pre-cancerous or developmentally abnormal lesions caused by HPV Types 6, 11, 16, 18, 31, 33, 45, 52 and 58.

The instruction for GARDASIL® 9 announced that HPV Types 16 and 18 are the cause of about 70% of cervical cancers, with the remaining 20% of cases attributed to Types 31, 33, 45, 52 and 58, therefor GARDASIL®9 prevents 90% of cervical cancers.

Industrial production of virus-like particles is critical to HPV vaccine development. The common systems for producing virus-like particles are mainly classified into the eukaryotic expression system and the prokaryotic expression system.

Commonly used eukaryotic expression systems include poxvirus expression systems, insect baculovirus expression systems, and yeast expression systems. The HPV L1 protein expressed in eukaryotic expression systems can be spontaneously assembled to virus-like particles as its natural conformation is less disrupted, but the yield thereof is low. The HPV L1 protein expressed in prokaryotic expression systems, mainly E. coli expression systems, is of high yields but mostly in the form of inclusion bodies, this form of protein can not be easily purified thus makes the production process complicated.

Therefore, there is still a need to obtain high yields of HPV virus-like particles.

### SUMMARY OF THE INVENTION

In one aspect, the present invention provides a chimeric papilloma virus L1 protein comprising, from its N-terminus to C-terminus orientation, a. an N-terminal fragment derived from L1 protein of the first papilloma virus type, said N-terminal fragment maintains the immunogenicity of the L1 protein of the corresponding human papilloma virus type; and b. a C-terminal fragment derived from L1 protein of the second papilloma virus type, said L1 protein of the second papilloma virus type has a better expression level and solubility compared to the L1 proteins of other types; wherein said chimeric papilloma virus L1 protein has the immunogenicity of the L1 protein of the first papilloma virus type.

In another aspect, the present invention provides a papilloma virus-like particle comprising a chimeric papilloma virus L1 protein.

In another aspect, the present invention provides an immunogenic composition for the prevention of papilloma virus-associated diseases or infections, comprising papilloma virus-like particles and adjuvants as previously described.

In another aspect, the present invention provides an isolated polynucleotide encoding a chimeric papilloma virus L1 protein.

In a further aspect, the present invention provides a vector comprising a polynucleotide encoding a chimeric papilloma virus L1 protein.

In a further aspect, the present invention provides a baculovirus comprising a polynucleotide encoding a chimeric papilloma virus L1 protein.

In another aspect, the present invention provides a host cell comprising said polynucleotides, said vectors, or said baculovirus as previously described.

In another aspect, the present invention provides a method of preparing a papilloma virus-like particle, comprising culturing host cells as previously described to express said chimeric papilloma virus L1 proteins and to assemble into virus-like particles; and purifying said papilloma virus-like particles.

### BRIEF DESCRIPTION OF THE DRAWINGS

FIG. 1A: Expression of L1 protein of HPV 6 L1: 33C. M: Marker; L: cell lysate; E-S: supernatant collected after centrifugation of the lysate.

FIG. 1B: Expression of L1 protein of HPV 11 L1: 33C. M: Marker; L: cell lysate; E-S: supernatant collected after centrifugation of the lysate.

FIG. 1C: Expression of L1 protein of HPV 16 L1: 33C. M: Marker; L: cell lysate; E-S: supernatant collected after centrifugation of the lysate.

FIG. 1D: Expression of L1 protein of HPV 18 L1: 33C. M: Marker; L: cell lysate; E-S: supernatant collected after centrifugation of the lysate.

FIG. 1E: Expression of L1 protein of HPV 31 L1: 33C. M: Marker; L: cell lysate; E-S: supernatant collected after centrifugation of the lysate.

FIG. 1F: Expression of L1 protein of HPV 35 L1: 33C. M: Marker; L: cell lysate; E-S: supernatant collected after centrifugation of the lysate.

FIG. 1G: Expression of L1 protein of HPV 39 L1: 59C. M: Marker; L: cell lysate; E-S: supernatant collected after centrifugation of the lysate.

FIG. 1H: Expression of L1 protein of HPV 45 L1: 33C. M: Marker; L: cell lysate; E-S: supernatant collected after centrifugation of the lysate.

FIG. 1I: Expression of L1 protein of HPV 51 L1: 33C. M: Marker; L: cell lysate; E-S: supernatant collected after centrifugation of the lysate.

FIG. 1J: Expression of L1 protein of HPV 52 L1: 33C. M: Marker; L: cell lysate; E-S: supernatant collected after centrifugation of the lysate.

FIG. 1K: Expression of L1 protein of HPV 56 L1: 33C. M: Marker; L: cell lysate; E-S: supernatant collected after centrifugation of the lysate.

FIG. 1L: Expression of L1 protein of HPV 58 L1: 33C. M: Marker; L: cell lysate; E-S: supernatant collected after centrifugation of the lysate.

FIG. 2A: Transmission electron microscopy of HPV 6 L1: 33C virus-like particles.

FIG. 2B: Transmission electron microscopy of HPV 11 L1: 33C virus-like particles.

FIG. 2C: Transmission electron microscopy of HPV 16 L1: 33C virus-like particles.

FIG. 2D: Transmission electron microscopy of HPV 18 L1: 33C virus-like particles.

FIG. 2E: Transmission electron microscopy of HPV 31 L1: 33C virus-like particles.

FIG. 2F: Transmission electron microscopy of HPV 35 L1: 33C virus-like particles.

FIG. 2G: Transmission electron microscopy of HPV 39 L1: 59C virus-like particles.

FIG. 2H: Transmission electron microscopy of HPV 45 L1: 33C virus-like particles.

FIG. 2I: Transmission electron microscopy of HPV 51 L1: 33C virus-like particles.

FIG. 2J: Transmission electron microscopy of HPV 52 L1: 33C virus-like particles.

FIG. 2K: Transmission electron microscopy of HPV 56 L1: 33C virus-like particles.

FIG. 2L: Transmission electron microscopy of HPV 58 L1: 33C virus-like particles.

FIG. 3: Expression of C-terminal truncated HPV 16L1 (1-474). M: Marker; L: cell lysates; E-S: supernatant collected after centrifugation of the lysates.

### SPECIFIC EMBODIMENTS

In one aspect, the present invention provides a chimeric papilloma virus L1 protein comprising, from its N-terminus to C-terminus orientation: a. an N-terminal fragment derived from L1 protein of the corresponding human papilloma virus type, said N-terminal fragment maintains the immunogenicity of the L1 protein of the corresponding type of HPV; and b. a C-terminal fragment derived from L1 protein of the second papilloma virus type, said L1 protein of the second papilloma virus type has a better expression level and solubility compared to L1 proteins of other types; wherein said chimeric papilloma virus L1 protein has the immunogenicity of L1 protein of the first papilloma virus type.

In one embodiment, said N-terminal fragment is a fragment obtained by truncating the C-terminus of the natural sequence of said L1 protein of the first papilloma virus type at any amino acid position within its α5 region, and a fragment having at least 98% identity therewith; said C-terminal fragment is a fragment obtained by truncating the N-terminus of the natural sequence of L1 protein of the second papilloma virus type at any amino acid position within its α5 region and functional variants resulting from further mutations, deletions and/or additions to the fragment.

In another embodiment, said N-terminal fragment has at least 98.5%, 99%, 99.5% or 100% identity to a fragment obtained by truncating the C-terminus of the natural sequence of said L1 protein of the first papilloma virus type at any amino acid position within its α5 region.

In one embodiment, said C-terminal fragment comprises one or plurality of nuclear localization sequences.

In one embodiment, said papilloma virus L1 protein is an HPV L1 protein.

In one embodiment, said L1 protein of the second papilloma virus type is selected from L1 proteins of HPV Types 1, 2, 3, 4, 6, 7, 10, 11, 13, 16, 18, 22, 26, 28, 31, 32, 33, 35, 39, 42, 44, 45, 51, 52, 53, 56, 58, 59, 60, 63, 66, 68, 73 or 82.

Preferably, said L1 protein of the second papilloma virus type is selected from L1 proteins of HPV Types 16, 28, 33, 59, or 68.

More preferably, said L1 protein of the second papilloma virus type is selected from L1 protein of HPV Type 33 or HPV Type 59.

In one embodiment, said L1 protein of the second papilloma virus type is an HPV Type 33 L1 protein, said C-terminal fragment is SEQ ID No: 2; or a fragment having a length of m1 amino acids, preferably a fragment covering amino acids 1-m1 position of SEQ ID No: 2; wherein m1 is an integer of 8-26; or said C-terminal fragment is SEQ ID No: 132; or a fragment having a length of m2 amino acids, preferably a fragment covering amino acids 1-m2 position of SEQ ID No: 132; wherein m2 is an integer of 13-31.

In one embodiment, the C-terminal fragment of the HPV Type 33 L1 protein has a nuclear localization sequence. In another embodiment, the C-terminal fragment of the HPV Type 33 L1 protein has two nuclear localization sequences. In some embodiments, the chimeric papilloma virus L1 protein comprises one or plurality of C-terminal fragments of the HPV Type 33 L1 protein. Said plurality of C-terminal fragments of the HPV Type 33 L1 protein may be the same or different. In one embodiment, the amino acid sequences with amino acid No: 7-8 (KR) and the amino acid sequences amino with amino acid No: 20-23 (KRKK) of SEQ ID No: 2 are nuclear localization sequences of the C-terminal fragments of the HPV Type 33 L1 protein.

In another embodiment, said L1 protein of the second papilloma virus type is the HPV Type 59 L1 protein, said C-terminal fragment is SEQ ID No: 13; or a fragment having a length of n amino acids, preferably covering amino acids 1-n position of SEQ ID No: 13; wherein n is an integer of 16-38.

In one embodiment, the C-terminal fragment of the HPV Type 59 L1 protein has a nuclear localization sequence. In another embodiment, the C-terminal fragment of the HPV Type 59 L1 protein has two nuclear localization sequences. In some embodiments, the chimeric papilloma virus L1 protein comprises one or plurality of C-terminal fragments of the HPV Type 59 L1 protein. Said plurality of C-terminal fragments of the HPV Type 59 L1 protein may be the same or different.

In one embodiment, said L1 protein of the first papilloma virus type is selected from L1 proteins of HPV Types 6, 11, 16, 18, 31, 35, 39, 45, 51, 52, 56 or 58.

In one embodiment, the C-terminus of said N-terminal fragment is connected to the N-terminus of said C-terminal fragment directly or via a linker.

The linker does not affect the immunogenicity of said N-terminal fragment and does not affect the expression level or solubility of the protein. In one embodiment, said N-terminal fragment and said C-terminal fragment are connected via a linker comprising 1, 2, 3, 4, 5, 6, 7, 8, 9 or 10 amino acids. In one embodiment, the linker is an artificial sequence. In another embodiment, the linker is a naturally occurring sequence in the HPV L1 protein. In another embodiment, the linker may be a partial sequence of the HPV Type 33 L1 protein. In another embodiment, the linker may be a partial sequence of the HPV Type 59 L1 protein.

In one embodiment, within the range of plus or minus 4 amino acid positions of the connection point when the C-terminus of said N-terminal fragment is connected to the N-terminus of said C-terminal fragment, there presents the following continuous amino acid sequence: RKFL (SEQ ID NO: 157); preferably, within the range of plus or minus 6 amino acid positions of the connection point, there presents the following continuous amino acid sequence: LGRKFL (SEQ ID NO: 158).

In one aspect, the present invention provides a papilloma virus-like particle, comprising a chimeric papilloma virus L1 protein as previously described. In one embodiment, the papilloma virus-like particle is the HPV virus-like particle, and in one embodiment, the HPV virus-like particle is the icosahedron comprising 72 pentamers of said chimeric HPV L1 protein. In one embodiment, the HPV virus-like particle has correctly formed disulfide bonds and thus has a good natural conformation. In one embodiment, the HPV virus-like particles self-assemble in an in vivo expression system.

In one aspect, the present invention provides an immunogenic composition for the prevention of papilloma virus-related disease or infections, comprising papilloma virus-like particles and adjuvants as previously described. Said prevention may be considered as a treatment and they can be used interchangeably.

In one aspect, the above-described immunogenic composition is administered to a subject. In one embodiment, the subject is a human. In one embodiment, the subject is a rabbit. In one embodiment, the subject is a dog.

In one aspect, the present invention provides an isolated polynucleotide encoding a chimeric papilloma virus L1 protein as previously described. In one embodiment, the polynucleotide is a codon-optimized polynucleotide for varieties of expression systems. In one embodiment, the polynucleotide is a codon-optimized polynucleotide for an insect baculovirus expression system.

In one aspect, the present invention provides a vector, comprising a polynucleotide as previously described. In one embodiment, the vector is a baculovirus vector. In one embodiment, the vector may be a transfer vector for a baculovirus expression system. In another embodiment, the vector may be an expression vector for a baculovirus expression system. In another embodiment, the vector may be a recombinant vector for the baculovirus expression system.

In one aspect, the present invention provides a baculovirus, comprising a polynucleotide as previously described.

In one aspect, the present invention provides a host cell, comprising the polynucleotide, the vector, or the baculovirus as previously described. In one embodiment, the host cell is an insect cell, preferably, said insect cell is selected from Sf9 cells, Sf21 cells, Hi5 cells and S2 cells.

In one aspect, the present invention provides a method for preparing papilloma virus-like particles as previously described, comprising: culturing the host cells as previously described to express said chimeric papilloma virus L1 proteins which is assembled into virus-like particles; and purifying said papilloma virus-like particles.

In one embodiment, the host cells are insect cells. In one embodiment, the host cells are Hi5 cells. In one embodiment, the chimeric papilloma L1 proteins are chimeric HPV L1 proteins that self-assemble into HPV virus-like particles in host cells. In one embodiment, the chimeric HPV L1 proteins self-assemble into HPV virus-like particles in host cells having an icosahedron comprising 72 pentamers of said chimeric HPV L1 proteins. In one embodiment, the HPV virus-like particles have correctly formed disulfide bonds and thus have a good natural conformation.

In one embodiment, the purification is carried out using cation exchange chromatography. In one embodiment, the purification is carried out using strong cation exchange chromatography. In another embodiment, the purification is carried out using weak cation exchange chromatography. In one embodiment, the purification is carried out using a combination of multiple cation exchange chromatography. In one embodiment, the purification is carried out using HS strong cation exchange chromatography. In another embodiment, the purification is carried out using MMA ion exchange chromatography. In another embodiment, purification is performed using HS-MMA two-step chromatography.

Papillomavirus L1 proteins expressed by eukaryotic expression systems can spontaneously assemble to virus-like particles, but the low expression level make them not suitable for mass production.

The sequences of the L1 proteins of each HPV type can be easily obtained from UniProt. For a given HPV type, the L1 protein can be derived from different strains, thus the amino acid sequence thereof may have multiple versions, any version of the natural sequence can be used in the present invention. It is possible that the sequence of the HPV L1 protein of a given type used during the conception and design of the present invention may differ from the sequence used in the following examples, but such differences do not affect the decisions and conclusions of the inventors.

It is generally accepted by those of skill in the art that the C-terminus of the L1 protein does not contain major neutralizing antigenic epitope and therefore attempts have been made to increase expression by truncating the C-terminus of the HPV L1 protein, for example Glaxo's US patent U.S. Pat. No. 6,361,778B1, in which the C-terminus of the HPV16 L1 protein is truncated by 1-34 amino acids, preferably 26 amino acids, states that the yield of VLP is increased by many-fold, preferably at least by 10 folds, and in particular by about 10 to 100 folds. Inspired by this, the inventors attempted to truncate the C-terminus of HPV 16 L1 protein by 31 amino acids and named the truncated protein as HPV 16 L1 (1-474). The protein is in high expression level but poor solubility, and is difficult to be extract and purified (see Comparative Example).

The poor solubility of the protein due to this truncation may be due to the deletion of the nuclear localization sequence located at the C-terminus, but the present invention is not binding to this speculation. During research and production, the inventor discovered that HPV Type 16 L1 protein, HPV Type 28 L1 protein, HPV Type 33 L1 protein, HPV Type 59 L1 protein and HPV Type 68 L1 protein have better expression levels and solubility than other HPV types L1 proteins. Inspired by the discovery, the inventors replaced the C-terminus of specific HPV types L1 proteins that are less extractable or less soluble with the C-terminus of specific HPV types L1 protein having better expression levels and solubility. That is, the inventors have constructed a chimeric protein: comprising, in the N-terminus to C-terminus orientation, an N-terminal fragment derived from L1 protein of the first papilloma virus type (e.g. HPV L1 protein), which provides the immunogenicity of the first papilloma virus type (e.g. HPV), and a C-terminal fragment derived from L1 protein of the second papilloma virus type (e.g. HPV L1 protein), which provides the features of better expression levels and solubility. These two fragments can be connected directly or via a linker.

The length of the N-terminal fragment of the HPV L1 protein appropriate for maintaining the immunogenicity of the L1 protein of the first HPV type and ensuring the formation of VLP is determined. The following reports relate to epitope studies of common HPV types.

Sunanda Baidya et al. reported that the epitopes of the L1 protein 48EEYDLQFIFQLCKITLTA65, 45RHGEEYDLQFIFQLCKITLTA65, 63LPDPNKF69, 79PETQRLVWAC88, 36PVPGQYDA43, 77YNPETQRLVWAC88, 188DTGYGAMD195, 36PVPGQYDATK45, 45KQDIPKVSAYQYRVFRV61, 130RDNVSVDYKQTQLCI144 and 49YSRHVEEYDLQFIF62 can be used as tools for designing HPV 16 and 18 vaccines (see Epitope design of L1 protein for vaccine production against Human Papilloma Virus types 16 and 18, Bioinformation 13 (3): 86-93 March 2017, incorporated herein by reference in its entirety).

Katharina Slupetzky et al. reported that the regions near aa 282-286 and 351-355 of HPV-16 contribute to the neutralization epitopes and that the latter is the immunodominant site (see Chimeric papilloma virus-like particles expressing a foreign epitope on capsid surface loops, Journal of General Virology (2001), 82, 2799-2804, incorporated herein by reference in its entirety).

Brooke Bishop et al. prepared the following three variants of the HPV 11, 16, 18 and 35 L1 proteins: deletion of 9 amino acids at its N terminus, deletion of α4 (corresponding to amino acid residues 404-436 of HPV 16), and deletion of 31 amino acids at its C terminus respectively, and reported that the former two could not be assembled into VLP, but this phenomenon has not been reported in the latter one (Crystal Structures of Four Types of Human Papillomavirus L1 Capsid Proteins UNDERSTANDING THE SPECIFICITY OF NEUTRALIZING MONOCLONAL ANTIBODIES, The Journal of Biological Chemistry, 282, 31803-31811. incorporated herein by reference in its entirety). Each the α-helix, β-fold sheets and Loop Region of each type of HPV L1 protein can be conveniently determined by sequence analysis software commonly used in the field. Wherein the α-helix regions contains the α1 Region, α2 Region, α3 Region, α4 Region and α5 Region. (The sequences in the following sequence alignment correspond with SEQ ID NOS. 170-173, respectively)

| | | |
|---|---|---|
| HPV16-L1 HPV35-L1 HPV11-L1 HPV18-L1 | 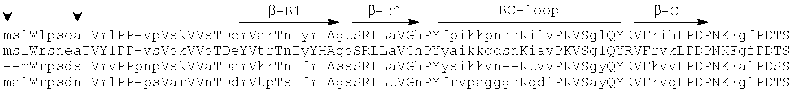 | 89 89 86 89 |
| HPV16-L1 HPV35-L1 HPV11-L1 HPV18-L1 | 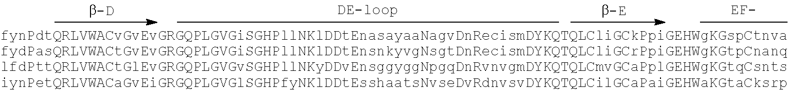 | 179 179 176 179 |
| HPV16-L1 HPV35-L1 HPV11-L1 HPV18-L1 | 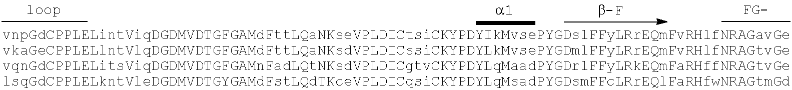 | 269 269 266 269 |
| HPV16-L1 HPV35-L1 HPV11-L1 HPV18-L1 | 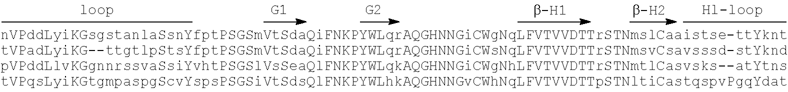 | 358 356 354 359 |
| HPV16-L1 HPV35-L1 HPV11-L1 HPV18-L1 | 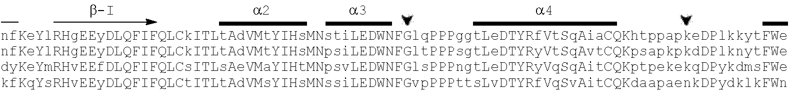 | 448 446 444 449 |
| HPV16-L1 HPV35-L1 HPV11-L1 HPV18-L1 | 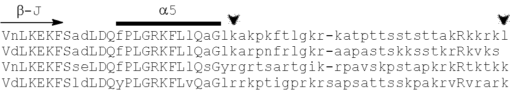 | 505 502 501 507 |

The inventors performed sequence alignments of L1 proteins of 14 HPV types (Types 6, 11, 16, 18, 31, 33, 35, 39, 45, 51, 52, 56, 58, and 59) and then performed secondary structure prediction according to the literature cited above (Crystal Structures of Four Types of Human Papillomavirus L1 Capsid Proteins UNDERSTANDING THE SPECIFICITY OF NEUTRALIZING MONOCLONAL ANTIBODIES, The Journal of Biological Chemistry, 282,31803-31811), the results of which are shown below, where the part between the downward-pointing arrows corresponds to the regions which had been deleted for preparing the variants in that literature. (The sequences in the following sequence alignments correspond with SEQ ID NOS: 174, 172, 175-178, 170-171, 179-182, 173, 183, respectively)

| | | |
|---|---|---|
| HPV6-L1 HPV11-L1 HPV52-L1 HPV58-L1 HPV33-L1 HPV31-L1 HPV16-L1 HPV35-L1 HPV51-L1 HPV56-L1 HPV39-L1 HPV59-L1 HPV18-L1 HPV45-L1 | 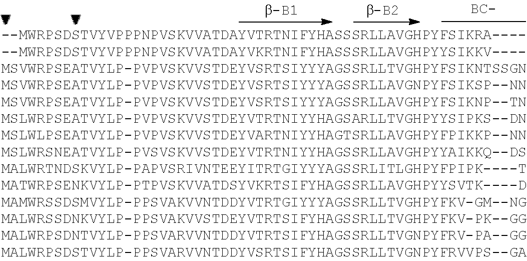 | 54 54 59 57 57 57 57 57 55 55 56 56 56 57 |
| HPV6-L1 HPV11-L1 HPV52-L1 HPV58-L1 HPV33-L1 HPV31-L1 HPV16-L1 HPV35-L1 HPV51-L1 HPV56-L1 HPV39-L1 HPV59-L1 HPV18-L1 HPV45-L1 | 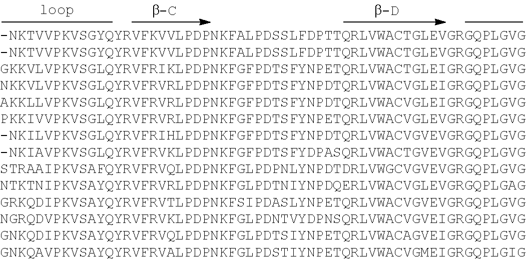 | 113 113 119 117 117 117 116 116 115 115 116 116 116 117 |
| HPV6-L1 HPV11-L1 HPV52-L1 HPV58-L1 HPV33-L1 HPV31-L1 HPV16-L1 HPV35-L1 HPV51-L1 HPV56-L1 HPV39-L1 HPV59-L1 HPV18-L1 HPV45-L1 | 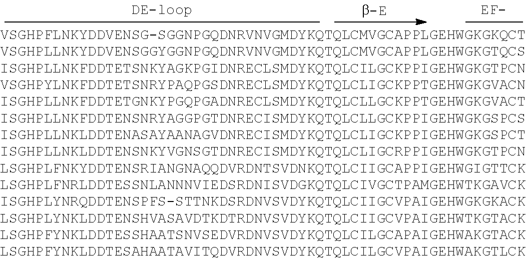 | 172 173 179 177 177 177 176 176 175 175 175 176 176 177 |
| HPV6-L1 HPV11-L1 HPV52-L1 HPV58-L1 HPV33-L1 HPV31-L1 HPV16-L1 HPV35-L1 HPV51-L1 HPV56-L1 HPV39-L1 HPV59-L1 HPV18-L1 HPV45-L1 | 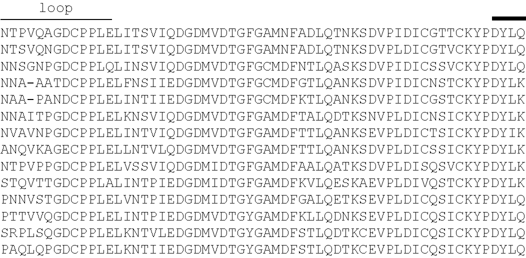 | 232 233 239 236 236 237 236 236 235 235 235 236 236 237 |
| HPV6-L1 HPV11-L1 HPV52-L1 HPV58-L1 HPV33-L1 HPV31-L1 HPV16-L1 HPV35-L1 HPV51-L1 HPV56-L1 HPV39-L1 HPV59-L1 HPV18-L1 HPV45-L1 | 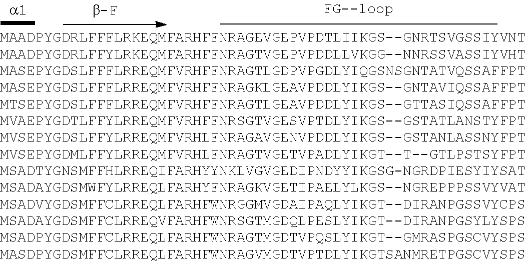 | 290 291 299 294 294 295 294 292 294 293 293 294 294 297 |
| HPV6-L1 HPV11-L1 HPV52-L1 HPV58-L1 HPV33-L1 HPV31-L1 HPV16-L1 HPV35-L1 HPV51-L1 HPV56-L1 HPV39-L1 HPV59-L1 HPV18-L1 HPV45-L1 | 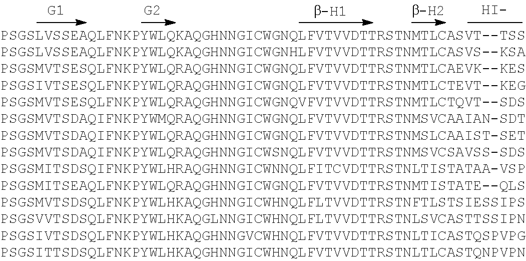 | 348 349 357 352 352 354 353 351 353 351 353 354 354 357 |
| HPV6-L1 HPV11-L1 HPV52-L1 HPV58-L1 HPV33-L1 HPV31-L1 HPV16-L1 HPV35-L1 HPV51-L1 HPV56-L1 HPV39-L1 HPV59-L1 HPV18-L1 HPV45-L1 | 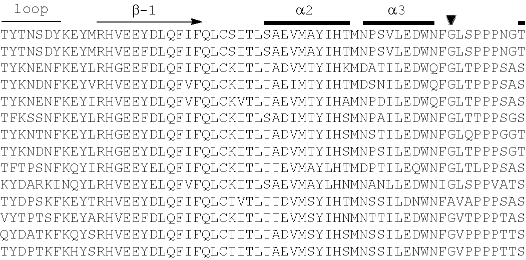 | 408 409 417 412 412 414 413 411 413 411 413 414 414 417 |
| HPV6-L1 HPV11-L1 HPV52-L1 HPV58-L1 HPV33-L1 HPV31-L1 HPV16-L1 HPV35-L1 HPV51-L1 HPV56-L1 HPV39-L1 HPV59-L1 HPV18-L1 HPV45-L1 | 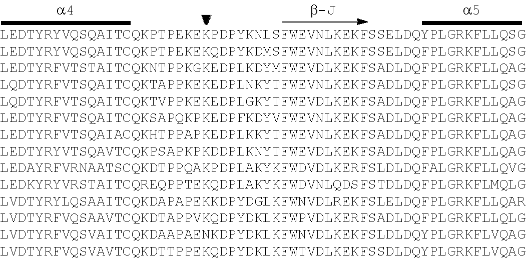 | 468 469 477 472 472 474 473 471 473 471 473 474 474 477 |
| HPV6-L1 HPV11-L1 HPV52-L1 HPV58-L1 HPV33-L1 HPV31-L1 HPV16-L1 HPV35-L1 HPV51-L1 HPV56-L1 HPV39-L1 HPV59-L1 HPV18-L1 HPV45-L1 | 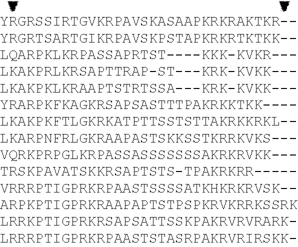 | 500 501 503 498 499 504 505 502 504 499 505 508 507 510 |

In addition to the methods used by the inventors for sequence alignments, protein secondary structure prediction software that can be used for prediction includes, but is not limited to:
1. JPred
2. ProtPredicct
3. PsiPred
4. SCRATCH-1D
5. Nnpredict
6. Predictprotein
7. SSPRED

In one embodiment of the present invention, the inventors determine the length of the N-terminal fragment of the HPV L1 protein derived from the first type in the following manner: the natural sequence of the L1 protein is truncated within its α5 region and nearby regions thereof, with the sequence from its N-terminus to the newly generated C-terminus within the α5 region retained. Such a truncated sequence ensures that it has the immunogenicity of the first type and is capable of forming VLP.

The N-terminal fragment of the HPV L1 protein derived from the first type can be further modified as long as it retains the immunogenicity of the first type and is capable of forming VLP.

The length of the C-terminal fragment of the HPV L1 protein derived from the second type is determined in the following manner: The natural sequence of the L1 protein is truncated within its α5 region and nearby regions thereof, then the sequence from the newly generated N-terminus within its α5 region to the C-terminus is retained. Such a truncated sequence does not have the major neutralizing antigenic epitope so that does not interfere with the immunogenicity of the resulting chimeric protein.

The C-terminal fragment of the HPV L1 protein derived from the second type can be further mutated, deleted and/or added, preferably retaining at least one of its nuclear localization sequences. Yang et al. predicted the nuclear localization sequences of 107 HPV subtypes (Yang et al. Predicting the nuclear localization signals of 107 types of HPV L1 proteins by bioinformatic analysis. Geno. Prot. Bioinfo. Vol. 4 No. 1 2006, incorporated herein by reference in its entirety). The nuclear localization sequences of each type of HPV L1 protein can be conveniently determined by sequence analysis software commonly used in the field.

The above-described connection of the N-terminal fragment to the C-terminal fragment occurs at the newly generated C-terminus of the former and at the newly generated N-terminus of the latter. This can be a direct connection or a connection via a linker. The site where the connection occurs is defined as the origin coordinate, the N-terminal side of the origin is regarded as minus while the C-terminal side as plus.

The following shows the sequence of amino acids 453-469 of the HPV6 L1 protein and the corresponding sequences of L1 proteins of other HPV types. These sequence overlap with their α5 region respectively. It can be seen these sequences are highly similar. The numbers in brackets indicate the positions of the last amino acid residue of the listed sequences, where for HPV Type 45, an additional 26 amino acids are present at the N-terminus of the L1 protein of some HPV Type 45 strains, while at the N-terminus of the L1 protein of other HPV Type 45 strains, the said additional 26 amino acids are not present, therefore the number is indicated as (478)+26.

| | HPV6 |
|---|---|
| (SEQ ID NO: 184) | |
| | ELDQYPLGRKFLLQSGY(469) |
| | HPV11 |
| (SEQ ID NO: 185) | |
| | ELDQFPLGRKFLLQSGY(470) |
| | HPV16 |
| (SEQ ID NO: 186) | |
| | DLDQFPLGRKFLLQAGL(474) |
| | HPV18 |
| (SEQ ID NO: 187) | |
| | DLDQYPLGRKFLVQAGL(475) |
| | HPV31 |
| (SEQ ID NO: 188) | |
| | DLDQFPLGRKFLLQAGY(475) |
| | HPV35 |
| (SEQ ID NO: 189) | |
| | DLDQFPLGRKFLLQAGL(472) |
| | HPV39 |
| (SEQ ID NO: 190) | |
| | ELDQFPLGRKFLLQARV(474) |
| | HPV45 |
| (SEQ ID NO: 191) | |
| | DLDQYPLGRKFLVQAGL(478)+26 |
| | HPV51 |
| (SEQ ID NO: 192) | |
| | DLDQFALGRKFLLQVGV(474) |
| | HPV52 |
| (SEQ ID NO: 193) | |
| | DLDQFPLGRKFLLQAGL(478) |
| | HPV56 |
| (SEQ ID NO: 194) | |
| | DLDQFPLGRKFLMQLGTRS(474) |
| | HPV58 |
| (SEQ ID NO: 195) | |
| | DLDQFPLGRKFLLQSGL(473) |
| | HPV33 |
| ((SEQ ID NO: 197) | |
| | DLDQFPLGRKFLLQAGL(473) |
| | KAKPKLKRAAPTSTRTSSAKRK |
| | KVKK |

HPV33 DLDQFPLGRKFLLQAGL (473) KAKPKLKRAAPTSTRTSSAKRKKVKK ((SEQ ID NO: 197) wherein KR at positions 480-481 and KRKK (SEQ ID NO: 197) at positions 493-496 are nuclear localization sequences.

HPV59 DLDQFPLGRKFLLQLGA (475) RPKPTIGPRKRAAPAPTSTPSPKRVKRRKSSRK (SEQ ID NO: 198) wherein RKR at positions 484-486 and KRVKRRK (SEQ ID NO: 199) at positions 498-504 are nuclear localization sequences.

In one embodiment of the present invention, the inventors have completed C-terminus substitutions of L1 proteins between different HPV types by taking advantage of sequence similarity of the α5 region and nearby regions between the HPV types.

In the most preferred embodiment of the present invention, the inventors have noted that L1 protein of each HPV type has a tetrapeptide RKFL (SEQ ID NO: 157) or, more advantageously, a hexapeptide LGRKFL (SEQ ID NO: 158), at a similar position. The inventors have skillfully exploited this highly conserved sequence to locate the chimeric protein connection point at any amino acid position within this oligopeptide. On one hand, the sequence starting from the N-terminus of the chimeric protein to RKFL (SEQ ID NO: 157) or LGRKFL (SEQ ID NO: 158) is identical to the sequence of the N-terminal fragment derived from L1 protein of the first HPV type, while on the other hand, the sequence starting from RKFL (SEQ ID NO: 157) or LGRKFL (SEQ ID NO: 158) to the C-terminus of the chimeric protein is identical to the sequence of the C-terminal fragment derived from L1 protein of the second type.

The chimeric protein thus produced maintains a high degree of similarity to the natural HPV L1 protein, and it can be expected to perform well in production and even in medical or prophylactic procedures thereafter.

It will be understandable for those skilled in the art that there are different strains with different natural sequences of a given HPV type, chimeric proteins constructed using different strains also fall within the scope of the present invention.

It will be understandable for those skilled in the art that because of the high degree of similarity between L1 protein of different HPV types, if, during the construction of the chimeric proteins, the N-terminal fragment derived from L1 protein of the first HPV type is extended by more amino acid residues towards the C-terminus, or the C-terminal fragment derived from L1 protein of the second HPV type is extended by more amino acid residues towards the N-terminus, it is also possible to form chimeric proteins that are structurally identical to the present invention due to identical or similar amino acids at the corresponding sites. The chimeric proteins thus formed also fall within the scope of the present invention.

It will be understandable for those skilled in the art that on the basis of the chimeric proteins of the above described embodiments, variants of the chimeric proteins may be formed by mutations, deletions and/or additions of amino acid residues. These variants are likely to have the immunogenicity of L1 protein of the first HPV type, can form VLP, and have a good yield and solubility. The chimeric proteins such formed also fall within the scope of the present invention.

### The Beneficial Effects of the Invention

The expression systems commonly used for producing virus-like particles are classified into eukaryotic expression systems and prokaryotic expression systems. The papilloma virus L proteins expressed by the eukaryotic expression systems can spontaneously assemble into viral-like particles, but have the disadvantage of low expression level thus are not suitable for mass production. The papilloma virus L protein expressed by the prokaryotic expression system requires in vitro processing to obtain virus-like particles because of often destroyed natural conformation, and has low yield, being difficult to be used in industrialization.

The present invention modifies the C-terminus of the L protein of the papilloma virus (e.g. human papilloma virus), for example by replacing it with the C-terminal fragment of HPV Type 16 L1 protein, HPV Type 28 L1 protein, HPV Type 33 L1 protein, HPV Type 59 L1 protein or HPV Type 68 L1 protein, thus can be used in expression systems (e.g. host cells, e.g. insect cells) to improve the expression level and the solubility of the papilloma virus L protein in expression systems (for example, host cells, e.g. insect cells). This can be used for the mass production of vaccines such as HPV vaccines.

The inventors found that the HPV Type 16 L1 protein, the HPV Type 28 L1 protein, the HPV Type 33 L1 protein, the HPV Type 59 L1 protein and the HPV Type 68 L1 protein have increased expression level and increased solubility compared to L1 proteins of other HPV types, and that said increased protein expression level and increased solubility was found to be depend on the C-terminal sequence of said HPV L1 protein. Among 107 HPV Type L1 proteins, most of them have nuclear localization sequences at the C-termini, and the C-terminal sequences have some similarities.

For papilloma virus L proteins that are currently inexpressible, very low in expression level or insoluble after expression, replacement of their C-terminal fragments with C-terminal fragments of the HPV Type 16 L1 protein, HPV Type 28 L1 protein, HPV Type 33 L1 protein, HPV Type 59 L1 protein or HPV Type 68 L1 protein makes it possible for soluble expression and subsequent purification. This strategy can be used for the mass production of polyvalent vaccines (e.g. HPV vaccines), making it possible to provide more comprehensive protection against a wide range of papilloma virus infections, especially HPV.

There needs to increase the expression level and the solubility of the HPV L1 protein in insect cells for mass production purpose. In addition, the virus-like particles assembled by the HPV L protein lack good conformation in yeast cells due to failure to form correct disulphide bonds.

For the HPV L1 proteins that are poorly expressed and insoluble in insect cells, a significant increase in expression level and solubility can be resulted after the modification of its C-terminal fragment into the C-terminal fragment of the HPV Type 33 or 59 L1 protein, thus they could be used for mass production of HPV vaccines.

For HPV L1 proteins that are better expressed and better soluble in insect cells compared to L1 proteins of other HPV types, such as HPV Type 16 protein, HPV Type 28 L1 protein, HPV Type 68 L1 protein, etc., there are needs to further improve the expression level and the solubility in order to achieve mass production of vaccines. In the present invention, for example, after modifying the C-terminal fragment of the HPV Type 16 L1 protein to the C-terminal fragment of the HPV Type 33 L1 protein, the expression level and the solubility of the modified chimeric HPV Type 16 protein are improved, which is conducive to the mass production of HPV vaccines.

To sum up, the chimeric HPV L1 proteins showed much higher expression level and solubility in insect cells compared to the unmodified HPV L1 protein. It can be used for the mass production of HPV vaccines. In addition, the chimeric HPV L1 proteins can correctly form disulfide bonds thus be assembled into HPV virus-like particles with good conformations in insect cells. This can improve the immunogenicity of HPV virus-like particles and trigger better immune responses.

### Definition

Unless otherwise stated, all technical and scientific terms used herein have the meanings normally understood by a person of ordinary skill in the art to which the invention belongs. For the convenience of understanding the present invention, the following terms are cited below for their ordinary meaning.

When used herein and in the attached claims, the singular forms “a/an”, “another” and “said/the” include the plural designations of the objects unless the context clearly indicates otherwise. Unless otherwise expressly stated, the terms “include/comprise/have”, “for example”, etc. are intended to convey inclusion rather than limitation.

The term “immunogenicity” refers to the ability of a substance, for example a protein or a peptide, to trigger an immune response, i.e. the ability to trigger the production of antibodies, in particular the ability to trigger humoral- or cell-mediated response.

The term “antibody” refers to an immunoglobulin molecule that binds an antigen. Antibodies may be polyclonal mixtures or monoclonal. Antibodies may be intact immunoglobulins of natural origin or of recombinant origin or may be immunoreactive portions of intact immunoglobulins. Antibodies may be present in a variety of forms including, for example, Fv, Fab′, F(ab′) 2 and as single chains.

The term “antigenicity” refers to the ability of a substance, for example a protein or a peptide, to trigger the production of antibodies that bind specifically to it.

The term “epitope” includes any protein determinant cluster that specifically binds to an antibody or T-cell receptor. Epitope determinants typically consist of chemically active surface groups (e.g. amino acids or sugar side chains, or combinations thereof) of the molecule and typically have specific three-dimensional structural characteristics as well as specific charge characteristics.

The terms “subtype” or “type” are used interchangeably herein to refer to genetic variant of virus that allows it can be recognized by the immune system as distinct antigen from type to type. For example, HPV 16 is immunologically distinguishable from HPV 33.

The term “HPV L1 protein”, as used herein, the term “HPV” and “human papilloma virus” refer to envelope-free double-stranded DNA viruses of the Papillomavirus family. Their genomes are circular and approximately 8 kilobase pairs in size. Most HPVs encode eight major proteins, six in the “early” region (E1-E2) and two in the “late” region (L1 (major capsid protein) and L2 (minor capsid protein)). Over 120 HPV types have been identified and they are labelled by numbers (e.g. HPV-16, HPV-18, etc.).

The term “HPV” or “HPV virus” refers to papilloma viruses of the Papillomaviridae Family, which are envelope-free DNA viruses with a double-stranded closed circular DNA genome of approximately 8 kb in size that is usually classified into three regions: (i) the early region (E), which contains six open reading frames E1, E2, E4-E7, encoding non-structural proteins related to viral replication, transcription and transformation, as well as open reading frames E3 and E8; (ii) the late region (L), which contains reading frames encoding the major capsid protein L1 and the minor capsid protein L2; and (iii) the long regulatory region (LCR), which does not encode any proteins but has the origin of replication and multiple transcription factor binding sites.

The terms “HPV L1 protein” and “HPV L2 protein” refer to proteins encoded by the late region (L) of the HPV gene and synthesized late in the HPV infection cycle. The L2 protein is the minor capsid protein. 72 L1 pentamers form the outer shell of the icosahedral HPV particles, which encloses the closed circular double-stranded DNA microchromosome.

The term “virus-like particle” refers to a hollow particle containing one or plurality of structural proteins of a virus, without viral nucleic acids.

“HPV pseudovirus” is an ideal model for in vitro neutralization of HPV, by taking advantages of the non-specific nucleic acid encapsulation property of HPV VLP, HPV pseudoviru is formed by wrapping free DNA or introducing an exogenous plasmid into a VLP composed of intracellularly-expressed HPV L1 and L2.

The “pseudovirus neutralization assay” is a method for evaluating the neutralizing activity of antibodies. After incubation of immunized animal serum with a certain amount of pseudovirus and then infection of the cells, the amount of the cells decreases when serum neutralizing antibodies increases, showing a linear negative correlation in a certain range. The neutralizing activity of antibodies in serum can therefore be assessed by measuring changes in the amounts of cells.

The term “fragment thereof” or “variant thereof” refers to a deletion, insertion and/or substitution of nucleotides or amino acids sequence of the present invention. Preferably, the fragment or variant of the polypeptide provided by the present invention is capable of triggering the humoral- and/or the cellular-immune response in animals or humans.

The term “chimeric” means that sequences of polypeptides or nucleotides derived from different parental molecules are connected together by —CO—NH— or 3′, 5′-phosphodiester bonds, respectively. Preferably, they are not spaced by additional linker sequences, but are directly adjacent to each other.

The term “truncation” refers to the removal of one or plurality of amino acids from the N- and/or C-terminus of a polypeptide or the deletion of one or plurality of amino acids from the interior of a polypeptide.

The term “nuclear localization sequence” refers to an amino acid sequence that directs the protein into the nucleus. In some HPV L1 proteins, two tight clusters of basic residues (i.e. nuclear localization sequences) (e.g. one is KRKR (SEQ ID NO: 200), KRKK (SEQ ID NO: 197), KRKRK (SEQ ID NO: 201), KRKKRK (SEQ ID NO: 202), KRVKRRK (SEQ ID NO: 199), etc. and the other is KR, RKR, KRK, etc.) have a spacer region of 10-14 amino acids between them. The above clusters of basic residues belong to nuclear localization sequences. In some other HPV L1 proteins, the nuclear localization sequence is a tight cluster of basic residues formed by arginines and/or lysines. Nuclear localization sequences include, but are not limited to, examples of clusters of basic residues as described above. See Jun Yang et al, Predicting the Nuclear Localization Signals of 107 Types of HPV L1 Proteins by Bioinformatic Analysis, Genomics, Proteomics & Bioinformatics Volume 4, Issue 1, 2006, Pages 34-41, the entire contents of which are incorporated herein by reference.

The term “functional variant” refers to a version of a polypeptide or a protein that retains the desired activities or characteristics after truncation, mutation, deletion and/or addition.

“Sequence identity” between two sequences of polypeptides or nucleic acids indicates the number of identical residues between said sequences as a percentage of the total number of residues, and is calculated based on the size of the smaller one of the compared molecules. When calculating the percentage identity, the sequences being compared are matched in such a way as to produce the maximum match between the sequences, with the vacant positions (if present) in the match being resolved by a specific algorithm. Preferred computer program methods for determining identity between two sequences include, but are not limited to, GCG program packages including GAP, BLASTP, BLASTN and FASTA (Altschul et al., 1990, J. Mol. Biol. 215:403-410). The above programs are publicly available from the National Center of Biotechnology Information (NCBI) and other sources. The well-known Smith Waterman Algorithm can also be used to determine the identity.

Non-critical amino acids can be conservatively substituted without affecting the normal function of the protein. Conservative substitution means replacing amino acids with chemically or functionally similar amino acids. Tables for conservative substitutions that provide similar amino acids are well known in the art. By way of example, in some embodiments, the groups of amino acids provided in Tables 1-3 are considered to be conservative substitutions for each other.

TABLE 1
In some embodiments, the selected groups of amino acids that are
considered to be conservative substitutions for each other

| | |
|---|---|
| Acid residues | D and E |
| Basic residues | K, R and H |
| Hydrophilic uncharged residues | S, T, N and Q |
| Aliphatic uncharged residues | G, A, V, L and I |
| Non-polar uncharged residues | C, M and P |
| Aromatic residues | F, Y and W |

TABLE 2
In some embodiments, other selected groups of amino acids considered
to be conservative substitutions for each other

| | |
|---|---|
| Group 1 | A, S and T |
| Group 2 | D and E |
| Group 3 | N and Q |
| Group 4 | R and K |
| Group 5 | I, L and M |
| Group 6 | F, Y and W |

TABLE 3
In some embodiments, other selected groups of amino acids considered
to be conservative substitutions for each other

| | |
|---|---|
| Group A | A and G |
| Group B | D and E |
| Group C | N and Q |
| Group D | R, K and H |
| Group E | I, L, M and V |
| Group F | F, Y and W |
| Group G | S and T |
| Group H | C and M |

The term “amino acids” refers to the twenty common naturally occurring amino acids. Naturally occurring amino acids include alanine (Ala; A), arginine (Arg; R), asparagine (Asn; N), aspartic acid (Asp; D), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y) and valine (Val; V).

The term “adjuvant” refers to a compound or mixture that enhances immune responses. In particular, a vaccine may comprise an adjuvant. Adjuvants for use in the present invention may include, but are not limited to, one or plurality of the following: mineral-containing adjuvant compositions, oil-emulsion adjuvants, saponin adjuvant formulations, derivatives of bacteria or microbes.

The term “vector” refers to a nucleic acid molecule capable of proliferating another nucleic acid connected to it. The term includes vectors as self-replicating nucleic acid structures and as vectors integrated into the genome of host cells into which the vector has been introduced. Certain vectors are capable of directing the expression of nucleic acids to which such vectors are operatively connected.

The term “host cell” refers to a cell into which an exogenous nucleic acid has been introduced, as well as to the progeny of such a cell. Host cells include “transformants” (or “transformed cells”), “transfectants” (or “transfected cells”) or “infectants” (or “infected cells”), each of which includes primary transformed, transfected or infected cells and the progeny derived from them. Such progeny may not be identical to the parental cells in terms of nucleic acid content and may contain mutations.

The administration amount is preferably a “prophylactically effective amount” (herein the prophylaxis may be considered as treatment and the two may be used interchangeably), which is sufficient to show benefit to the individual.

### EXAMPLES

### Example 1 Construction of chimeric genes

### Example 1.1: Construction of a Chimeric Gene with the C-Terminus of HPV6 L1 Substituted with the C-Terminus of HPV33 L1

1.1.1 Construction of pFB-HPV6 L1 as the Template

The HPV6 L1 gene with the KpnI and XbaI cleavage sites at both ends of the synthesized sequences was synthesized by Thermo Fisher [formerly Invitrogen (Shanghai) Trading Co.], its sequence is shown in SEQ ID NO: 5. The plasmid pcDNA3-HPV6-L1 comprising the nucleotide sequence encoding amino acid 1-500 of HPV6 L1 was obtained by ligating the synthesized gene fragment with pcDNA3 vector (distributor: Thermo Fisher) at KpnI and XbaI cleavage sites.

The obtained pcDNA3-HPV6-L1 plasmid was subjected to double enzyme digestion with KpnI and XbaI to obtain a gene fragment of HPV6 L1 (1-500). The fragment was then ligated with the KpnI/XbaI double digested pFastBac™1 vector (distributor: Thermo Fisher) to obtain a rod vector containing the HPV6 L1 (1-500) gene fragment, named as pFB-HPV6 L1.

1.1.2 Construction of pFB-HPV33 L1 as the Template

The HPV33 L1 gene with the Kpn I and XbaI cleavage sites at both ends of the synthesized sequences was synthesized by Thermo Fisher [formerly Invitrogen (Shanghai) Trading Co.), its sequence is shown as SEQ ID NO: 6. The plasmid pcDNA3-HPV33-L1 comprising the nucleotide sequence encoding amino acids 1-499 of HPV33 L1 was obtained by ligating the synthesized gene fragment with pcDNA3 vector (distributor: Thermo Fisher) at Kpn I and XbaI cleavage sites.

The pcDNA3-HPV33-L1 plasmid was subjected to double enzyme digestion with KpnI and XbaI to obtain a fragment of the HPV33 L1 (1-499) gene. The fragment was then ligated to the KpnI and XbaI double digested pFastBac™1 vector (distributor: Thermo Fisher) to obtain a rod vector containing the HPV33 L1 (1-499) gene fragment, named as pFB-HPV33 L1.

1.1.3 Construction of pFB-HPV6 L1: 33C

Chimeric gene with HPV6 L1 C-terminus substituted with HPV33 L1 C-terminus: the constructed recombinant plasmid pFB-HPV6 L1 was used as the gene template to amplify a 1426 bp gene fragment using primers F1 and R1, the primer sequence F1 is shown in SEQ ID No: 7 and R1 is shown in SEQ ID No: 8.

This gene fragment contains a fragment encoding amino acids 1-469 of HPV6 L1, 10 bases overlapping with the gene fragment encoding amino acids 474-499 of HPV33 L1, and a fragment of the KpnI digest site (GGTAC\hat{ }C), the amplified sequence is shown in SEQ ID No: 9.

PCR amplification parameters: pre-denaturation at 94° C. for 5 min; denaturation at 98° C. for 10 s, annealing at 69° C. for 15 s, 1 kb/1 min at 72° C., for 30 cycles; extension at 72° C. for 5 min; end at 16° C.

The recombinant plasmid pFB-HPV33 L1 was used as the gene template to amplify a gene fragment of 101 bp in length using primers F2 and R2, the primer sequence of F2 is shown in SEQ ID No: 10 and the primer sequence of R2 is shown in SEQ ID No: 11.

This gene fragment contains a gene fragment encoding 26 C-terminal amino acids (474-499) of HPV33 L1, a 10 bp base overlapping with the gene fragment encoding the C-terminus of amino acids 1-469 of HPV6 L1 and the XbaI (T\hat{ }CTAGA) digest site, the amplified sequence is shown in SEQ ID No: 12.

PCR amplification parameters: pre-denaturation at 94° C. for 5 min; denaturation at 98° C. for 10 s, annealing at 69° C. for 15 s, 1 kb/1 min at 72° C., for 30 cycles; extension at 72° C. for 5 min; end at 16° C.

PCR ligating sequences:

The ligating primers were F1 and R2, and the fragments amplified using the above primers (amplified fragments of F1 and R1, amplified fragments of F2 and R2) were used as templates.

PCR ligating parameters: pre-denaturation at 94° C. for 5 min; denaturation at 98° C. for 10 s, annealing at 52° C. for 15 s, 72° C. for 1 kb/1 min, for 5 cycles; denaturation at 98° C. for 10 s, annealing at 68° C. for 15 s, 72° C. for 1 kb/1 min, for 25 cycles; extension at 72° C. for 5 min; end at 16° C.

The final result was SEQ ID NO: 4, a nucleotide sequence encoding amino acids 1-469 of HPV6 L1 and 26 C-terminal amino acids of HPV33 L1 (aa 474-499), with KpnI and XbaI cleavage sites at both ends (hereafter referred to as the ligating sequence).

The recombinant plasmid pFB-HPV6 L1: 33C was obtained by double digesting the pFastBac™1 vector and the ligating sequence fragment with KpnI+XbaI enzymes and cloning the ligating sequence into the pFastBac™1 vector to obtain pFB-HPV6 L1: 33C, which is a chimeric gene with the C-terminus of HPV6 L1 substituted by the C-terminus of HPV33 L1.

### Example 1.2 Construction of Chimeric Gene with the C-Terminus of HPV11 L1 Substituted by the C-Terminus of HPV33 L1

The experimental methods and procedures were the same as in Example 1.1, see Appendix 2 for relevant sequences.

### Example 1.3 Construction of a Chimeric Gene with the C-Terminus of HPV16 L1 Substituted by the C-Terminus of HPV33 L1

The experimental method and procedure were the same as in Example 1.1, see Appendix 3 for relevant sequences.

### Example 1.4 Construction of a Chimeric Gene with the C-Terminus of HPV18 L1 Substituted by the C-Terminus of HPV33 L1

The experimental method and procedure were the same as in Example 1.1, see Appendix 4 for relevant sequences.

### Example 1.5 Construction of a Chimeric Gene with the C-Terminus of HPV31 L1 Substituted by the C-Terminus of HPV33 L1

The experimental method and procedure were the same as in Example 1.1, see Appendix 5 for relevant sequences.

### Example 1.6 Construction of a Chimeric Gene with the C-Terminus of HPV35 L1 Substituted by the C-Terminus of HPV33 L1

The experimental methods and procedures were the same as in Example 1.1, see Appendix 6 for relevant sequences.

### Example 1.7 Construction of Chimeric Gene with the C-Terminus of HPV39 L1 Substituted by the C-Terminus of HPV59L1

1.7.1 Construction of pFB-HPV39 L1 Used as the Template

The HPV39 L1 gene with the KpnI and XbaI cleavage sites at both ends of the synthesized sequences was synthesized by Thermo Fisher [formerly Invitrogen (Shanghai) Trading Co.), its sequence is shown as SEQ ID NO: 83. The plasmid pcDNA3-HPV39-L1 containing a nucleotide sequence encoding amino acids 1-505 of HPV39 L1 was obtained by ligating the synthesized gene fragment with pcDNA3 vector (distributor: Thermo Fisher) at Kpn I and XbaI cleavage sites.

The pcDNA3-HPV39-L1 plasmid was subjected to double digestion with KpnI and XbaI to obtain a fragment of the HPV39 L1 (1-505) gene. The fragment was then ligated to the KpnI and XbaI double digested pFastBac™1 vector (distributor: Thermo Fisher) to obtain a rod vector containing the HPV39 L1 (1-505) gene fragment, named as pFB-HPV39 L1.

1.7.2 Construction of pFB-HPV59L1 as the Template

The HPV59L1 gene was synthesized by Thermo Fisher [formerly Invitrogen (Shanghai) Trading Co.) to obtain plasmid pcDNA3-HPV59-L1 containing the nucleotide sequence encoding amino acids 1-508 of HPV59L1.

The pcDNA3-HPV59-L1 plasmid was double digested with KpnI and XbaI to obtain a fragment of the HPV59L1 (1-508) gene. The fragment was then ligated to the KpnI/XbaI double digested pFastBac™1 vector (distributor: Thermo Fisher) to obtain a rod vector containing the HPV59L1 (1-508) gene fragment, named as pFB-HPV59L1.

1.7.3 Construction of pFB-HPV39 L1: 59C

Chimeric gene with HPV39 L1 C-terminus substituted with HPV59L1 C-terminus: The constructed recombinant plasmid pFB-HPV39 L1 was used as the gene template to amplify a 1428 bp gene fragment using primers F1 and R1, the primer sequence F1 is shown in SEQ ID No: 85 and the primer sequence R1 is shown in SEQ ID No: 86.

This fragment contains a fragment encoding amino acids 1-469 of HPV39 L1, a 12-base overlapping with a fragment encoding amino acids 471-508 of HPV59L1 and a segment of the KpnI digest site (GGTAC\hat{ }C), the amplified sequence is shown in SEQ ID No: 87.

PCR amplification parameters: pre-denaturation at 94° C. for 5 min; denaturation at 98° C. for 10 s, annealing at 69° C. for 15 s, 1 kb/1 min at 72° C., for 30 cycles; extension at 72° C. for 5 min; end at 16° C.

The recombinant plasmid pFB-HPV59L1 was used as the gene template to amplify a gene fragment of 139 bp in length using primers F2 and R2. The primer sequence F2 is shown in SEQ ID No: 88 and R2 is shown in SEQ ID No: 89.

This gene fragment contains a gene fragment encoding 38 C-terminal amino acids (471-508) of the HPV59L1, a 12 bp base overlapping with the gene fragment encoding amino acids 1-469 of HPV39 L1 and the XbaI (T\hat{ }CTAGA) digest site, and the amplified sequence is shown in SEQ ID No: 90.

PCR amplification parameters: pre-denaturation at 94° C. for 5 min; denaturation at 98° C. for 10 s, annealing at 69° C. for 15 s, 1 kb/1 min at 72° C. for 30 cycles; extension at 72° C. for 5 min; end at 16° C.

### PCR Ligating Sequence

The ligating primers were F1 and R2, and the fragments amplified by using the above primers (F1 and R1 amplified fragments, F2 and R2 amplified fragments) were used as templates.

PCR ligating parameters: pre-denaturation at 94° C. for 5 min; denaturation at 98° C. for 10 s, annealing at 52° C. for 15 s, 72° C. for 1 kb/1 min, for 5 cycles; denaturation at 98° C. for 10 s, annealing at 68° C. for 15 s, 72° C. for 1 kb/1 min, for 25 cycles; extension at 72° C. for 5 min; end at 16° C.

The final result was SEQ ID NO: 82, a nucleotide sequence encoding amino acids 1-469 of HPV39 L1 and 38 C-terminal amino acids of HPV59L1 (471-508) with KpnI and XbaI enzyme cleavage sites at both ends (hereafter referred to as the ligating sequence).

The recombinant plasmid pFB-HPV39 L1: 59C was obtained by double digesting the pFastBac™1 vector and the ligating sequence fragment with KpnI+XbaI enzymes and the ligating sequence was cloned into the pFastBac™1 vector o obtain pFB-HPV39 L1: 59C, which is a chimeric gene with the C-terminus of HPV39 L1 substituted by the C-terminus of HPV59L1.

### Example 1.8 Construction of Chimeric Gene with the C-Terminus of HPV45L1 Substituted by the C-Terminus of HPV33 L1

The experimental methods and procedures were the same as in Example 1.1, see Appendix 8 for relevant sequences.

### Example 1.9 Construction of a Chimeric Gene with the C-Terminus of HPV51L1 Substituted by the C-Terminus of HPV33 L1

The experimental methods and procedures were the same as in Example 1.1, see Appendix 9 for relevant sequences.

### Example 1.10 Construction of a Chimeric Gene with the C-Terminus of HPV52L1 Substituted by the C-Terminus of HPV33 L1

The experimental methods and procedures were the same as in Example 1.1, see Appendix 10 for relevant sequences.

### Example 1.11 Construction of a Chimeric Gene with the C-Terminus of HPV56L1 Substituted by the C-Terminus of HPV33 L1

The experimental methods and procedures were the same as in Example 1.1, see Appendix 11 for relevant sequences.

### Example 1.12 Construction of a Chimeric Gene with the C-Terminus of HPV58L1 Substituted by the C-Terminus of HPV33 L1

The experimental methods and procedures were the same as in Example 1.1, see Appendix 12 for relevant sequences.

### Example 2 Recombinant Baculovirus Packaging

### Example 2.1: HPV6 L1: 33C Recombinant Baculovirus Packaging of

The recombinant plasmid of pFB-HPV6 L1: 33C constructed in Example 1 was identified and sequenced to be correct, and was transformed into DH10Bac bacteria competent cells (Bac-to-Bac® kit, purchased from Thermo Fisher), incubated at 37° C. for proliferation, and incubated in a flat dish for streak culture. White colonies was selected, incubated overnight. The bacterial culture was collected and the recombinant baculovirus DNA was extracted using alkaline lysis method.

The recombinant baculovirus DNA was transfected into insect cells SF9 using a cationic transfection reagent (purchased from Sino Biological) to package the recombinant baculovirus virulent strains. The procedure was as follows:
a. SF9 cells at log phase were inoculated in dishes at a density of 0.6×10⁶ cell/dish. The dish inoculated with SF9 cells were left at room temperature for 2 hrs. for the cell's adhering to the dish wall.
b. 20 μL of extracted plasmid Bacmid DNA was added to 200 μL Grace's Medium (no serum, no additives, purchased from Gibico) and mixed and inverted 5 times.
c. 25 μL of 0.2× TF1 (transfection reagent, purchased from Sino Biological) was added dropwise to 200 μL of Grace's Medium and mixed gently.
d. Mixed b and c. Incubated at room temperature for 15-45 min.
e. During the incubation of DNA with cellfectin (purchased from Sino Biological), the cell supernatant was discarded and 0.8 mL of Grace Medium (serum additive free) was added into the dish.
f. The incubated mixture of DNA and transfection reagent of d was added to the dish dropwise.
g. Incubated at 27° C. for 2 hr.
h. Cell culture medium was discarded and 2.5 mL complete growth media/dish (SCD6 SF+10% FBS) (SCD6 SF was purchased from Sino Biological, FBS was purchased from Gibico) a was added.
i. Culture was performed at 27° C. for 7 days and whether the viral infection occurred was observed.

Virus supernatant was collected after visible lesions were observed in the transfected cells, typically after 7-11 days of culturing. The viral supernatant, i.e., the P1 generation virus strain of HPV6 L1: 33C is collected aseptically with a pipette. SF9 cells, at a density of 2×10⁶ cells/mL, were infected using P1 generation virus strain of HPV6 L1: 33C at a ratio of 1:50 (V/V), cultured at 27° C. for 3 days, and centrifuged at 1000 g±200 g for 10 min at room temperature. The collected virus supernatant was the P2 generation virus and could be used for infecting the host cells and production.

### Example 2.2: Packaging of HPV11 L1: 33C Recombinant Baculovirus

The experimental methods and procedures were the same as in Example 2.1.

### Example 2.3: Packaging of HPV 16L1: 33C Recombinant Baculovirus

The experimental methods and procedures were the same as those of Example 2.1.

### Example 2.4: Packaging of HPV18 L1: 33C Recombinant Baculovirus

The experimental methods and procedures were the same as those of Example 2.1.

### Example 2.5: Packaging of HPV31 L1: 33C Recombinant Baculovirus

The experimental methods and procedures were the same as those of Example 2.1.

### Example 2.6: Packaging of HPV35 L1: 33C Recombinant Baculovirus

The experimental methods and procedures were the same as those of Example 2.1.

### Example 2.7: Packaging of HPV39 L1: 59C Recombinant Baculovirus

The experimental methods and procedures were the same as those of Example 2.1.

### Example 2.8: Packaging of HPV45 L1: 33C Recombinant Baculovirus

The experimental methods and procedures were the same as those of Example 2.1.

### Example 2.9: Packaging of HPV51 L1: 33C Recombinant Baculovirus

The experimental methods and procedures were the same as those of Example 2.1.

### Example 2.10: Packaging of HPV52 L1: 33C Recombinant Baculovirus

The experimental methods and procedures were the same as those of Example 2.1.

### Example 2.11: Packaging of HPV56 L1: 33C Recombinant Baculovirus

The experimental methods and procedures were the same as those of Example 2.1.

### Example 2.12: Packaging of HPV58 L1: 33C Recombinant Baculovirus

The experimental methods and procedures were the same as those of Example 2.1.

### Example 3 Expression of Chimeric Proteins

### Example 3.1: Expression of HPV6 L1: 33C

High Five cells were infected with baculovirus containing the HPV6 L1: 33C recombinant gene obtained in Example 2 at a ratio of 1:200 (V/V), and the cell precipitate was collected by centrifugation at 1000 g±100 g at room temperature. The cells were broken up by sonication at low temperature for 3 min, centrifuged at >10,000 g for 10 min and the supernatant was collected for SDS-PAGE. Lane 1: Marker (The marker is a mixture of 7 purified proteins with molecular weights ranging from 14.4 kDa to 116 kDa, produced by Thermo Scientific); Lane 2: cell lysate; Lane 3: supernatant of the lysate collected by centrifugation.

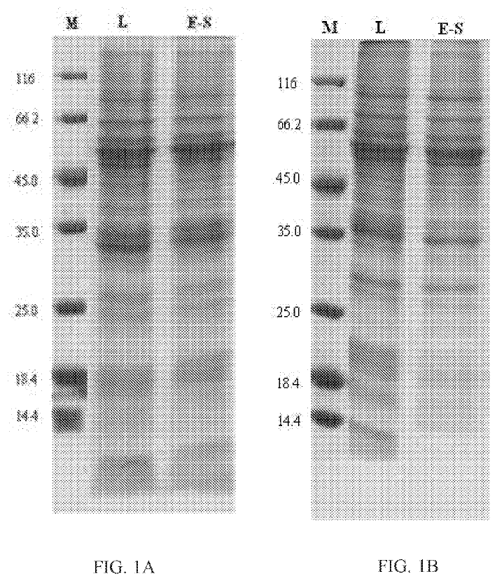

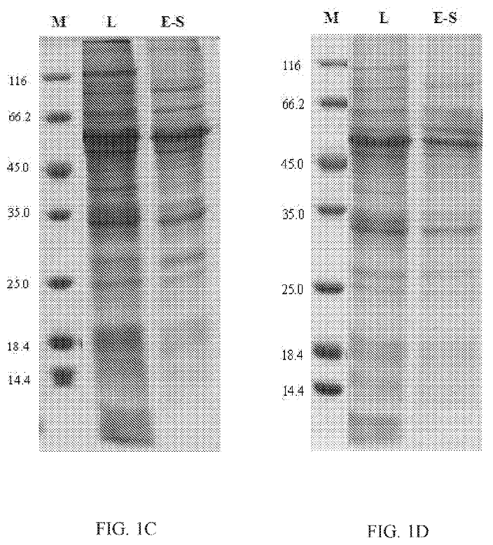

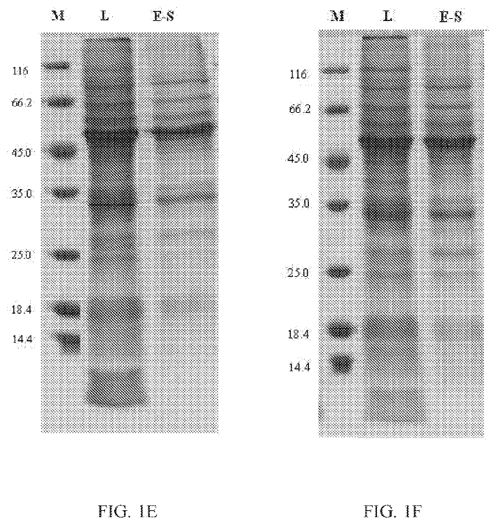

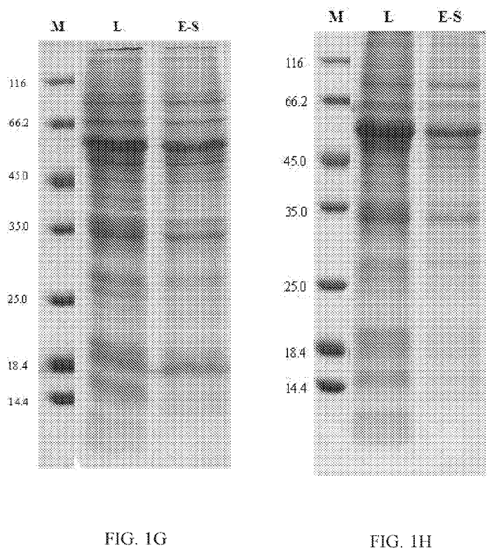

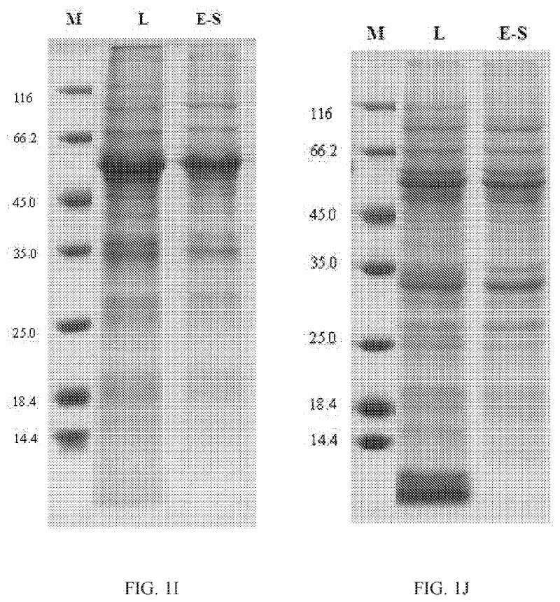

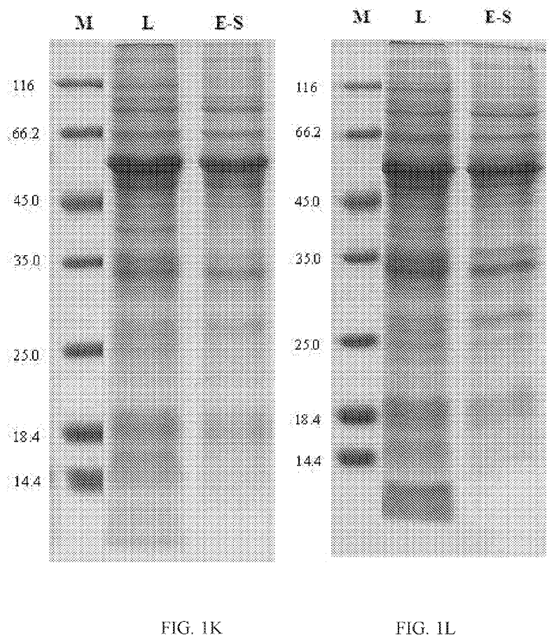

The result is shown in FIG. 1A. The HPV6 L1: 33C L1 protein prepared by this method has a yield of >100 mg/L and a protein size of approximate 56 KD, which can be used for mass production.

### Example 3.2: Expression and Production of HPV11 L1: 33C

The experimental methods and procedures were the same as in Example 3.1.

The result is shown in FIG. 1B. The HPV11 L1: 33CL1 protein prepared by this method has a yield of >100 mg/L and a protein size of approximate 56 KD, which can be used for mass production.

### Example 3.3: Expression and Production of HPV 16L1: 33C

The experimental methods and procedures were the same as in Example 3.1.

The result is shown in FIG. 1C. The HPV 16L1: 33CL1 protein prepared by this method has a yield of >100 mg/L and a protein size of approximate 56 KD, which can be used for mass production.

### Example 3.4: Expression and Production of HPV18 L1: 33C

The experimental methods and procedures were the same as in Example 3.1.

The result is shown in FIG. 1D. The HPV18 L1: 33C L1 protein prepared method has a yield of >100 mg/L and a protein size of approximate 56 KD, which can be used for mass production.

### Example 3.5: Expression and Production of HPV31 L1: 33C

The experimental methods and procedures were the same as in Example 3.1.

The result is shown in FIG. 1E. The HPV31 L1: 33C L1 protein prepared by this method has a yield of >100 mg/L and a protein size of approximate 56 KD, which can be used for mass production.

### Example 3.6: Expression and Production of HPV35 L1: 33C

The experimental methods and procedures were the same as in Example 3.1.

The result is shown in FIG. 1F. The HPV35 L1: 33C L1 protein prepared by this method has a yield of >100 mg/L and a protein size of approximate 56 KD, which can be used for mass production.

### Example 3.7: Expression and Production of HPV39 L1: 59C

The experimental methods and procedures were the same as in Example 3.1.

The result is shown in FIG. 1G. The HPV39 L1: 59C L1 protein prepared by this method has a yield of >100 mg/L and a protein size of approximate 56 KD, which can be used for mass production.

### Example 3.8: Expression and Production of HPV45 L1: 33C

The experimental methods and procedures were the same as in Example 3.1.

The result is shown in FIG. 1H. The HPV45 L1: 33CL1 protein prepared by this method has a yield of >100 mg/L and a protein size of approximate 56 KD, which can be used for mass production.

### Example 3.9: Expression and Production of HPV51 L1: 33C

The experimental methods and procedures were the same as in Example 3.1.

The result is shown in FIG. 1I. The HPV51 L1: 33C L1 protein prepared by this method has a yield of >100 mg/L and a protein size of approximate 56 KD, which can be used for mass production.

### Example 3.10: Expression and Production of HPV52 L1: 33C

The experimental methods and procedures were the same as in Example 3.1.

The result is shown in FIG. 1J. The HPV52 L1: 33C L1 protein prepared by this method has a yield of >100 mg/L and a protein size of approximate 56 KD, which can be used for mass production.

### Example 3.11: Expression and Production of HPV56 L1: 33C

The experimental methods and procedures were the same as in Example 3.1.

The result is shown in FIG. 1K. The HPV56 L1: 33C L1 protein prepared by this method has a yield of >100 mg/L and a protein size of approximate 56 KD, which can be used for mass production.

### Example 3.12: Expression and Production of HPV58 L1: 33C

The experimental methods and procedures were the same as in Example 3.1.

The result is shown in FIG. 1L. The HPV58 L1: 33C L1 protein prepared by this method has a yield of >100 mg/L and a protein size of approximate 56 KD, which can be used for mass production.

### Example 4 Preparation of purified virus-like particles

### Example 4.1: Preparation of purified HPV6 L1: 33C virus-like particles

The HPV6 L1: 33C virus-like particles were purified by a two-step chromatography method, i.e. HS-MMA method, the supernatant collected in Example 3 was purified, and finally, high purity virus-like particles were obtained.

First step chromatography:

Medium: POROS 50 HS strong cation exchange media produced by Thermo Fisher was used.

Medium volume: 150 mL of media volume, 30 mL/min of linear flow rate.

Chromatography conditions: equilibration buffer (pH 6.2, the salt concentration is 50 mM phosphate, 0.5 M NaCl); wash buffer (the salt concentration is 50 mM phosphate, 0.75 M NaCl, pH 6.2).

The chromatography column was first equilibrated with 5 CV of equilibration buffer and then the sample was loaded. After loading, the column was then eluted with 5 CV of equilibration buffer and wash buffer, respectively, to remove the protein impurities.

Elution conditions: a 50 mM phosphate buffer containing 50 mM arginine hydrochloride, pH 6.2, with an elution salt concentration being of 1.25 M NaCl, was used.

Second step chromatography.

Medium: MMA ion exchange media produced by Bestchrom (Shanghai) Biosciences Co., Ltd was used.

Medium volume: media volume is150 mL, while linear flow rate is 30 mL/min.

Chromatography conditions: equilibration buffer: 50 mM PB, 1.25 M NaCl, pH 6.2. The chromatography column was first equilibrated with 4 CV equilibration buffer and then the sample was loaded. After loading, protein impurities were rinsed off with 5 CV equilibration buffer and then the target protein was eluted with elution buffer and collected.

Elution conditions: 100 mM NaAC, 150 mM NaCl, 0.01% Tween 80, pH 4.5.

### Example 4.2: Preparation of Purified HPV11 L1: 33C Virus-Like Particles

The experimental methods and procedures were the same as in Example 4.1.

### Example 4.3: Purification and Preparation of HPV 16L1: 33C Virus-Like Particles

The experimental methods and procedures were the same as in Example 4.1.

### Example 4.4: Purification and Preparation of HPV18 L1: 33C Virus-Like Particles

The experimental methods and procedures were the same as in Example 4.1.

### Example 4.5: Purification and Preparation of HPV31 L1: 33C Virus-Like Particles

The experimental methods and procedures were the same as in Example 4.1.

### Example 4.6: Preparation of Purified HPV35 L1: 33C Virus-Like Particles

The experimental methods and procedures were the same as in Example 4.1.

### Example 4.7: Preparation of Purified HPV39 L1: 59C Virus-Like Particles

The experimental methods and procedures were the same as in Example 4.1.

### Example 4.8: Preparation of Purified HPV45 L1: 33C Virus-Like Particles

The experimental methods and procedures were the same as in Example 4.1.

### Example 4.9: Preparation of Purified HPV51 L1: 33C Virus-Like Particles

The experimental methods and procedures were the same as in Example 4.1.

### Example 4.10: Preparation of Purified HPV52 L1: 33C Virus-Like Particles

The experimental methods and procedures were the same as in Example 4.1.

### Example 4.11: Preparation of Purified HPV56 L1: 33C Virus-Like Particles

The experimental methods and procedures were the same as in Example 4.1.

### Example 4.12: Preparation of Purified HPV58 L1: 33C Virus-Like Particles

The experimental methods and procedures were the same as in Example 4.1.

### Example 5 Morphological Observation of Virus-Like Particles

### Example 5.1: Morphological Observation of HPV6 L1: 33C Virus-Like Particles

10 μL sample was taken for transmission electron microscopy. The sample was fixed onto a carbon coated copper grid for 2 min, the rest liquid was absorbed off with filter paper, and then stained twice with phosphotungstic acid (Beijing Electron Microscopy China Technology Co., Ltd., concentration 2%, pH 6.5) for 30 seconds each time, the rest staining solution was absorbed off with filter paper, left the sample for drying, then performed transmission electron microscopy observation. The transmission electron microscope (Brand: Hitachi, Model No.: H-7650) was 80 KV with a magnification of 80,000×.

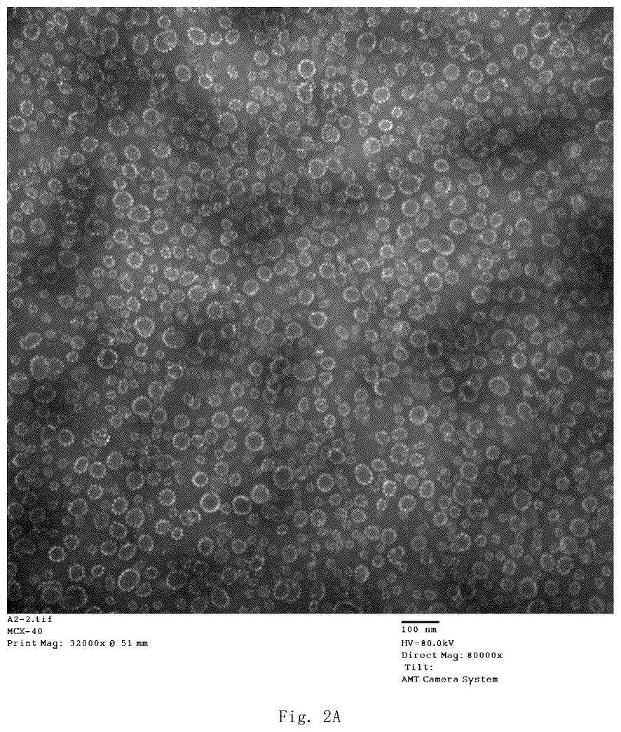

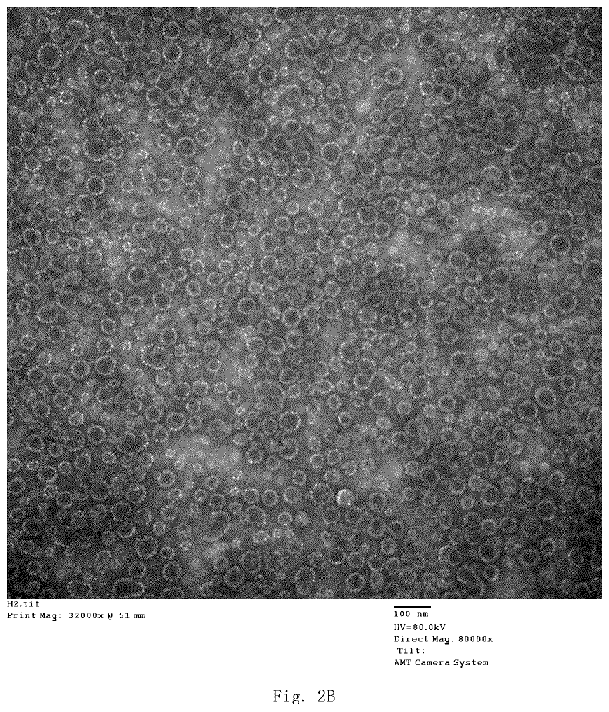

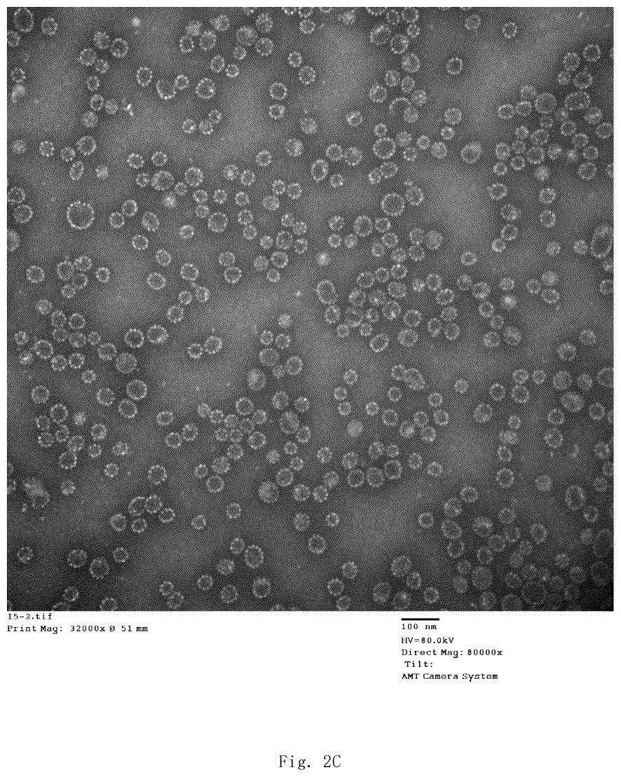

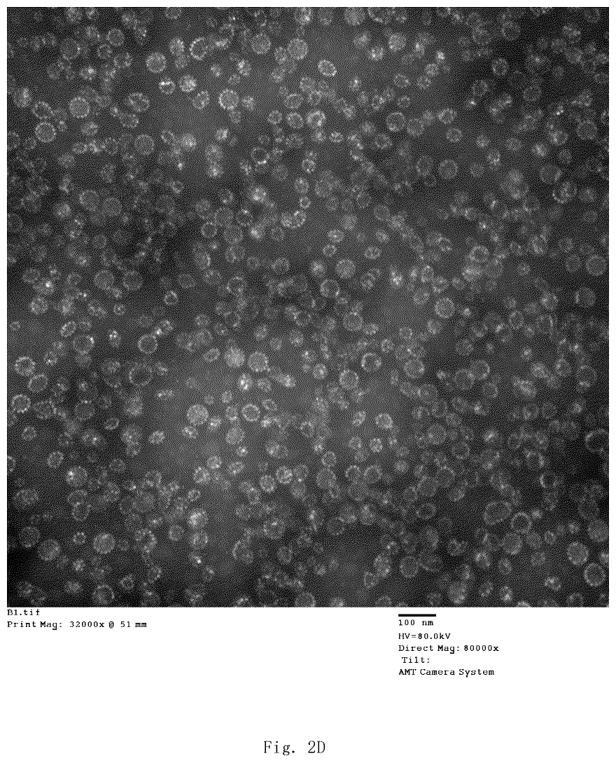

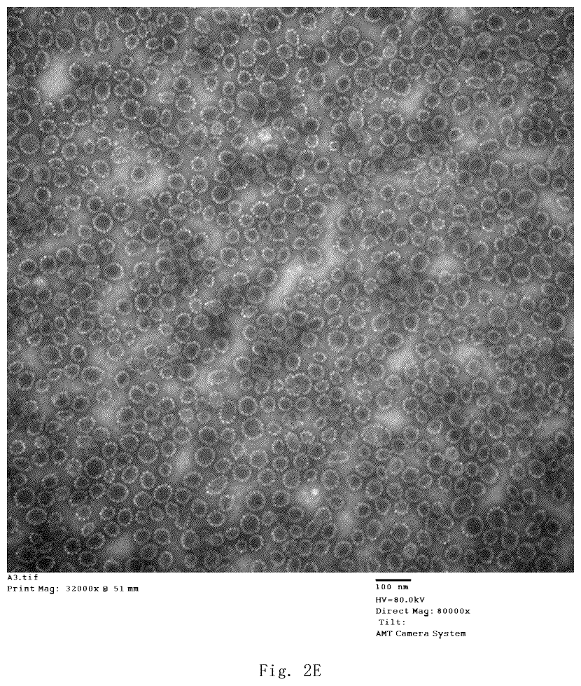

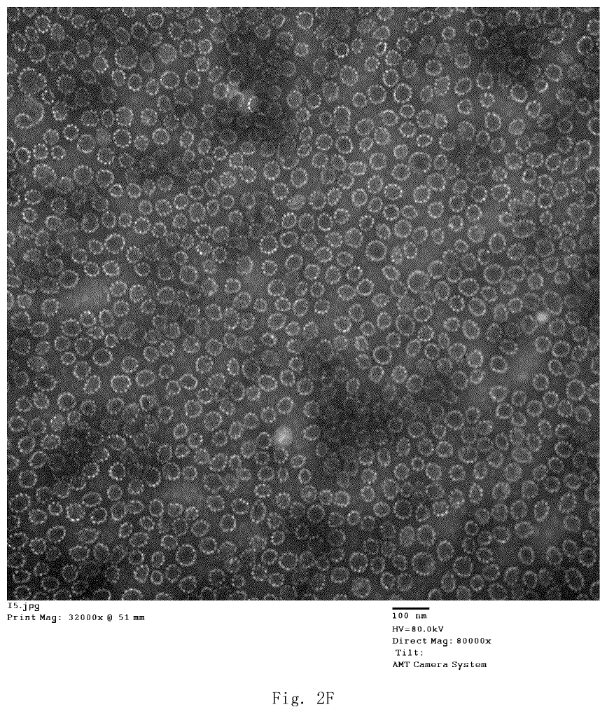

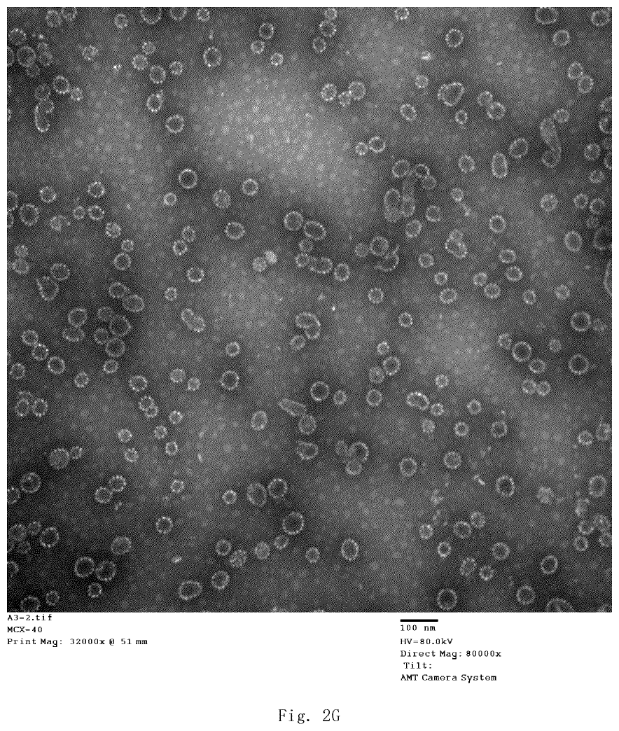

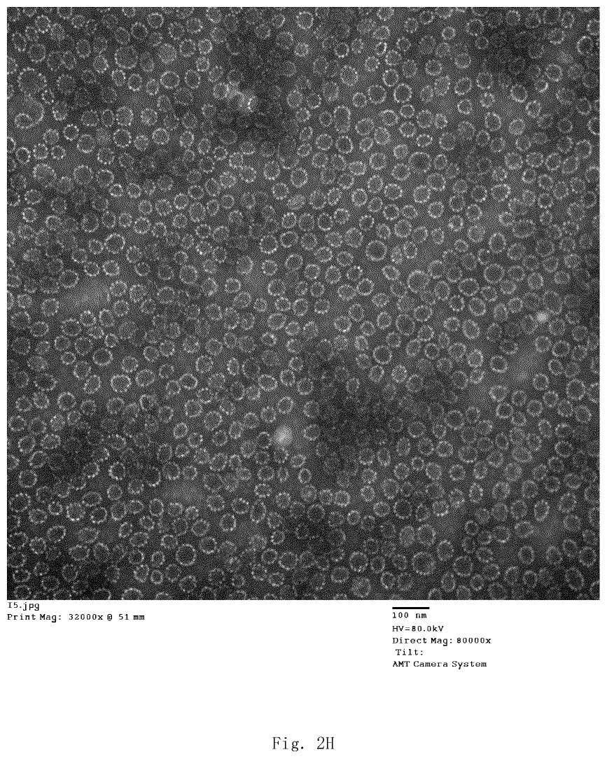

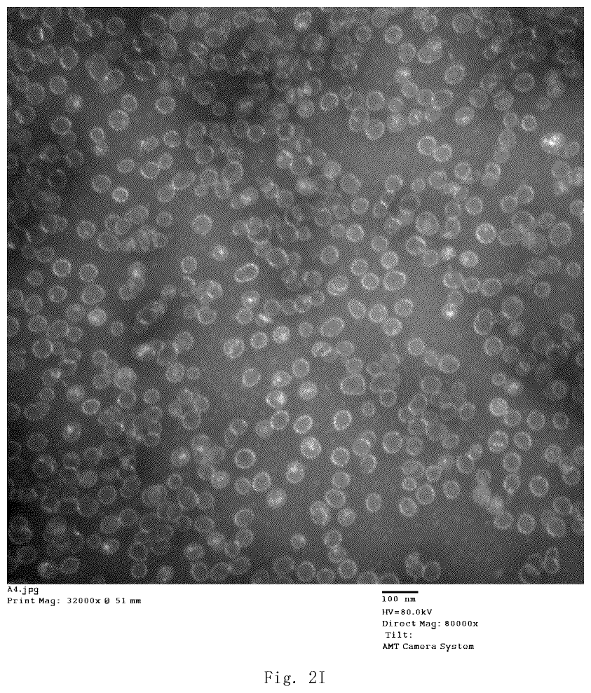

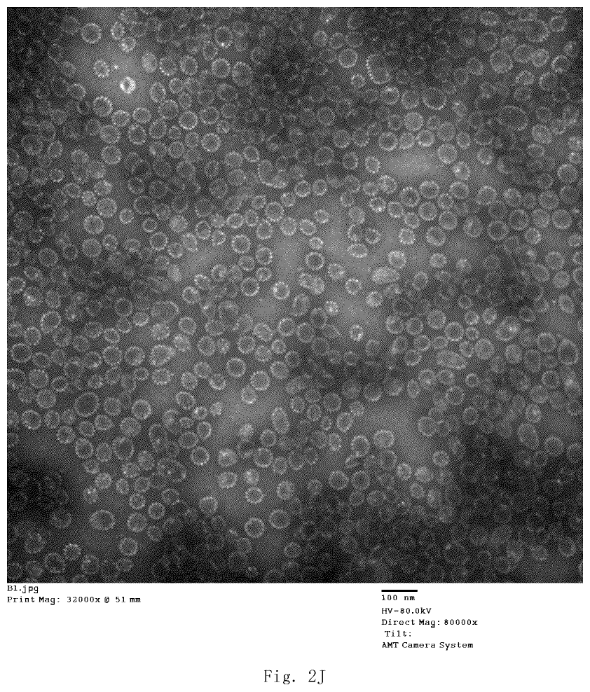

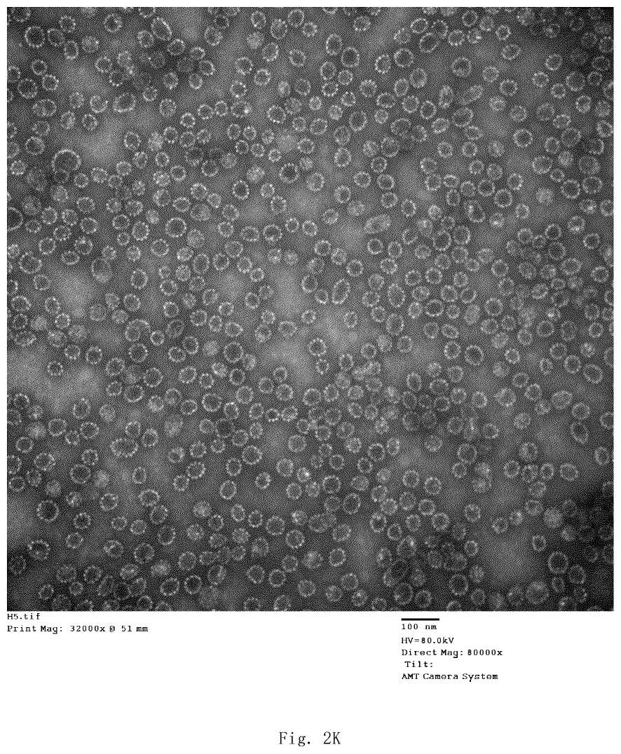

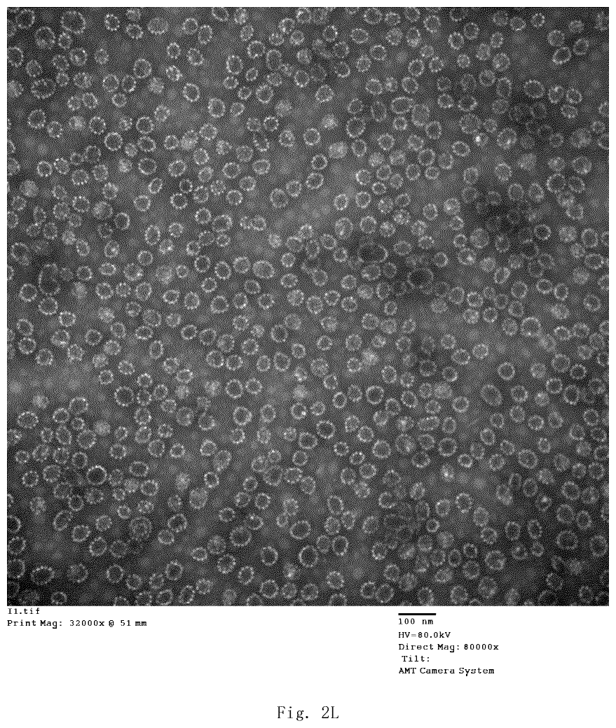

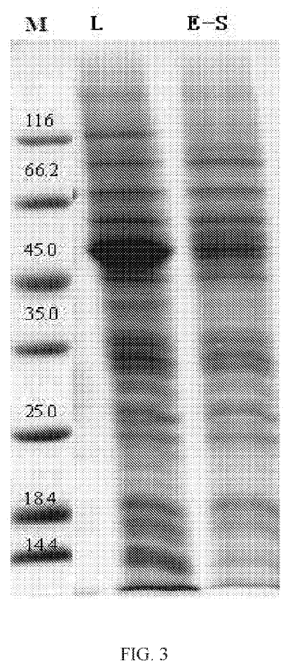

The electron microscopy observation is shown in FIG. 2A. As can be seen in FIG. 2A, the C-terminal-modified HPV6 L1: 33C can form uniform-sized virus-like particles with an average diameter of approximate 60 nm.

### Example 5.2: Morphological Observation of HPV11 L1: 33C Virus-Like Particles

The experimental methods and procedures were the same as in Example 5.1.

The electron microscopy observation is shown in FIG. 2B. As can be seen in FIG. 2B, the C-terminal-modified HPV11 L1: 33C can form uniform-sized virus-like particles, with an average diameter of approximate 60 nm.

### Example 5.3: Morphological Observation of HPV 16L1: 33C Virus-Like Particles

The experimental methods and procedures were the same as in Example 5.1.

The electron microscopy observation is shown in FIG. 2C. As can be seen in FIG. 2C, the C-terminal-modified HPV 16L1: 33C can form uniform-sized virus-like particles with an average diameter of approximate 60 nm.

### Example 5.4: Morphological Observation of HPV18 L1: 33C Virus-Like Particles

The experimental methods and procedures were the same as in Example 5.1.

The electron microscopy observation is shown in FIG. 2D. As can be seen in FIG. 2D, the C-terminal-modified HPV18 L1: 33C can form uniform-sized virus-like particles with an average diameter of approximate 60 nm.

### Example 5.5: Morphological Observation of HPV31 L1: 33C Virus-Like Particles

The experimental methods and procedures were the same as in Example 5.1.

The electron microscopy observation is shown in FIG. 2E. As can be seen in FIG. 2E, the C-terminal-modified HPV31 L1: 33C can form uniform-sized virus-like particles with an average diameter of approximate 60 nm.

### Example 5.6: Morphological Observation of HPV35 L1: 33C Virus-Like Particles

The experimental methods and procedures were the same as in Example 5.1.

The electron microscopy observation is shown in FIG. 2F. As can be seen in FIG. 2F, the C-terminally modified HPV35 L1: 33C can form uniform-sized virus-like particles with an average diameter of approximate 60 nm.

### Example 5.7: Morphological Observation of HPV39 L1: 59C Virus-Like Particles

The experimental methods and procedures were the same as in Example 5.1.

The electron microscopy observation is shown in FIG. 2G. As can be seen in FIG. 2G, the C-terminal-modified HPV39 L1: 59C can form uniform-sized virus-like particles with an average diameter of approximate 60 nm.

### Example 5.8: Morphological Observation of HPV45 L1: 33C Virus-Like Particles

The experimental methods and procedures were the same as in Example 5.1.

The electron microscopy observation is shown in FIG. 2H. As can be seen in FIG. 2H, the C-terminal-modified HPV45 L1: 33C can form uniform-sized virus-like particles with an average diameter of approximate 60 nm.

### Example 5.9: Morphological Observation of HPV51 L1: 33C Virus-Like Particles

The experimental methods and procedures were the same as in Example 5.1.

The electron microscopy observation is shown in FIG. 2I. As can be seen in FIG. 2I, the C-terminal-modified HPV51 L1: 33C can form uniform-sized virus-like particles with an average diameter of approximate 60 nm.

### Example 5.10: Morphological Observation of HPV52 L1: 33C Virus-Like Particles

The experimental methods and procedures were the same as in Example 5.1.

The electron microscopy observation is shown in FIG. 2J. As can be seen in FIG. 2J, the C-terminal-modified HPV52 L1: 33C can form uniform-sized virus-like particles with an average diameter of approximate 60 nm.

### Example 5.11: Morphological Observation of HPV56 L1: 33C Virus-Like Particles

The experimental methods and procedures were the same as in Example 5.1.

The electron microscopy observation is shown in FIG. 2K. As can be seen in FIG. 2K, the C-terminal-modified HPV56 L1: 33C can form uniform-sized virus-like particles with an average diameter of approximate 60 nm.

### Example 5.12: Morphological Observation of HPV58 L1: 33C Virus-Like Particles

The experimental methods and procedures were the same as in Example 5.1.

The electron microscopy observation is shown in FIG. 2L. As can be seen in FIG. 2L, the C-terminal-modified HPV58 L1: 33C can form uniform-sized virus-like particles with an average diameter of approximate 60 nm.

### Example 6 Immunogenicity Evaluation of Virus-Like Particles in Animals

### Example 6.1: Immunogenicity Evaluation of HPV6 L1: 33C Virus-Like Particles in Animals

### 6.1.1 Modeling of Pseudovirus-Neutralizing Cells

As HPV is difficult to be cultured in vitro and has a strong host specificity, it is difficult to be reproduced in organisms other than humans, thus there is a lack of suitable animal models. Therefore, there is a need to establish suitable and effective in vitro neutralization experimental models for the evaluation of vaccine immunoprotectivity.

HPV pseudovirus is an ideal model for HPV in vitro neutralization: Thanks to the HPV VLP's characteristic of non-specifically encapsulating nucleic acids, HPV pseudovirus can be formed from the VLPs, composed of HPV L1 and L2 expressed in cells, by encapsulating free DNA or introducing exogenous plasmid.

The immunogenicity of immunized animal serum samples was analyzed by pseudovirus neutralization assay. The animal immunized with HPV6 virus-like particles can produce neutralizing antibodies against HPV6 which can neutralize HPV6 pseudovirus. When the immunized animal serum is incubated with a certain amount of pseudovirus and then infects cells, the number of cells capable of expressing GFP fluorescence decreases when neutralization antibodies in the serum increases, showing a linear negative correlation in a certain range, so the neutralizing activity of antibodies in the serum can be evaluated by detecting the change in the number of cells expressing GFP.

Construction method of pseudovirus: The HPV6 pCMV3-3-HPV6 L1+L2 (L1 sequence was from Uniprot P69898, L2 sequence was from Uniprot Q84297) plasmid (purchased from Sino Biological) and the fluorescent plasmid (PSEU-GFP Spark, purchased from Sino Biological) were co-transfected into 293 FT adherent cells (purchased from Thermo Fisher). The specific methods refer to the published literature (Pastrana D V, Buck C B, Pang Y S, Thompson C D, Castle P E, FitzGerald P C, Kjaer S K, Lowy D R, Schiller J T. Reactivity of human sera in a sensitive, high-throughput pseudovirus-based papilloma virus neutralization assay for HPV16 and HPV18. [J] Virology 2004,321:205-216.). The pseudovirus supernatant was collected and aliquoted, and stored in a −80° C. refrigerator for stock.

6.1.2 Immunoprotective evaluation of HPV6 L1: 33C virus-like particles in animals Immunization procedures in mice:
HPV6 L1: 33C virus-like particles were adsorpted onto aluminium phosphate adjuvant, mixed, and used to immunize mice at a dose of 0.15 μg/200 μL per mouse, 10 mice in total. The mice were immunized with the diluted samples on Days 0, 7 and 21, with control mice immunized with blank serum. Blood was collected from the eyes of the mice on Day 28 and the sera were isolated for pseudovirus neutralization titers assay.

EC50 assay of mice:
The murine serum was inactivated at 56° C. for 30 minutes, centrifuged at 6000 g, 5 mins, and the supernatant was collected for assaying. 4-8 hours prior to the assay, 293 FT cells were inoculated at a density of 15,000 cells/well into 96-well plates and incubated at 37° C. in a CO₂ incubator with 5% CO₂. The post-immune murine serum and blank control serum were serial diluted with neutralizing media respectively, then mixed with the HPV6 pseudovirus prepared in 6.1 at a volume ratio of 1:1, incubated at 2-8° C. for 1 hrs., then 100 L/well of the mixture were added to 293 FT cells, which had been inoculated for 4-8 hrs in advance. Each sample was in replicate, and blank serum control group, pseudovirus positive control group and pseudovirus negative control group were used. The cells infected by pseudovirus were incubated at 37° C. in a CO₂ incubator with 5% CO₂ for 62-96 hrs, fluorescence scanning photographed and counted in an ELISPOT analyser (Model No.: S6 Universal-V Analyzer, Manufacturer: CTL). On the basis of the neutralization inhibition of each murine serum sample, the maximum dilution of the serum at 50% neutralization inhibition was calculated for each murine serum sample according to the Reed-Muench method, i.e. the half efficacy dilution EC50.

The results of the HPV6 serum pseudovirus neutralization titer assay are detailed in Table 4.

TABLE 4
EC50 of Serum Neutralization Titer Assay in Mice (GMT ± SEM)

| HPV Type | EC₅₀ Value |
|---|---|
| Type 6 | 4948 ± 1831 |

Notes:
1. number of animals, N = 10.
2. GMT (Geometric Mean Titer): geometric mean titer.
3. SEM (Standard Error of Mean): standard error.

The above evaluation results show that the HPV6 L1: 33C virus-like particles prepared by the present invention have good immunogenicity and can produce neutralizing antibodies with high titers in animals, which can be used to prepare into a vaccine for preventing HPV infections.

### Example 6.2: Evaluation of Immunogenicity of HPV11 L1: 33C Virus-Like Particles in Animals

The experimental methods and procedures were the same as in Example 6.1. The L1 sequence was from Uniprot P04012 and the L2 sequence was from Uniprot P04013.

The results of HPV11 serum pseudovirus neutralization titer assay are detailed in Table 5.

TABLE 5
EC50 of Serum Neutralization Titer Assay in Mice (GMT ± SEM)

| HPV Type | EC₅₀ Value |
|---|---|
| Type 11 | 15024 ± 8400 |

The above evaluation results show that the HPV11 L1: 33C virus-like particles prepared by the present invention have good immunogenicity, can produce high titers of neutralizing antibodies in animals, and can be used to prepare into a vaccine for the prevention of HPV infection.

### Example 6.3: Evaluation of Immunogenicity of HPV 16L1: 33C Virus-Like Particles in Animals

The experimental methods and procedures were the same as in Example 6.1. The L1 sequence was from Uniprot P03101 and the L2 sequence was from Uniprot P03107.

The results of HPV16 serum pseudovirus neutralization titer assay are detailed in Table 6.

TABLE 6
EC50 of Serum Neutralization Titer Assay in Mice (GMT ± SEM)

| HPV Type | EC₅₀ Value |
|---|---|
| Type 16 | 32449 ± 7224 |

The above evaluation results show that the HPV 16L1: 33C virus-like particles prepared by the present invention have good immunogenicity and can produce high titers of neutralizing antibodies in animals, which can be used to prepare into a vaccine for the prevention of HPV infection.

### Example 6.4: Evaluation of Immunogenicity of HPV18 L1: 33C Virus-Like Particles in Animals

The experimental methods and procedures were the same as in Example 6.1. The L1 sequence was from Uniprot Q80B70 and the L2 sequence was from Uniprot P06793.

The results of HPV18 serum pseudovirus neutralization titer assay are detailed in Table 7.

TABLE 7
EC50 of Serum Neutralization Titer Assay in Mice (GMT ± SEM)

| HPV Type | EC₅₀ Value |
|---|---|
| Type 18 | 18480 ± 4051 |

The above evaluation results show that the HPV18 L1: 33C virus-like particles prepared by the present invention have good immunogenicity, can produce high titers of neutralizing antibodies in animals, and can be used to prepare into a vaccine for preventing HPV infection.

### Example 6.5: Evaluation of Immunogenicity of HPV31 L1: 33C Virus-Like Particles in Animals

The experimental methods and procedures were the same as in Example 6.1. The L1 sequence was from Uniprot P17388 and the L2 sequence was from Uniprot P17389.

The results of HPV31 serum pseudovirus neutralization titer assay are detailed in Table 8.

TABLE 8
EC50 of Serum Neutralization Titer Assay in Mice (GMT ± SEM)

| HPV Type | EC₅₀ Value |
|---|---|
| Type 31 | 5210 ± 1147 |

The above evaluation results show that the HPV31 L1: 33C virus-like particles prepared by the present invention have good immunogenicity, can produce high titers of neutralizing antibodies in animals, and can be used to prepare into a vaccine for preventing HPV infection.

### Example 6.6: Evaluation of Immunogenicity of HPV35 L1: 33C Virus-Like Particles in Animals

The experimental methods and procedures were the same as in Example 6.1. The L1 sequence was from Uniprot P27232 and the L2 sequence was from Uniprot P27234.

The results of HPV35 serum pseudovirus neutralization titer assay are detailed in Table 9.

TABLE 9
EC50 of Serum Neutralization Titer Assay in Mice (GMT ± SEM)

| HPV Type | EC₅₀ Value |
|---|---|
| Type 35 | 2293 ± 1448 |

The above evaluation results show that the HPV35 L1: 33C virus-like particles prepared by the present invention have good immunogenicity, can produce high titers of neutralizing antibodies in animals, and can be used to prepare into a vaccine for preventing HPV infection.

### Example 6.7: Immunogenicity Evaluation of HPV39 L1: 59C Virus-Like Particles in Animals

The experimental methods and procedures were the same as in Example 6.1. The L1 sequence was from Uniprot P24838 and the L2 sequence was from Uniprot P24839.

The results of HPV39 serum pseudovirus neutralization titer assay are detailed in Table 10.

TABLE 10
EC50 of Serum Neutralization Titer Assay in Mice (GMT ± SEM)

| HPV Type | EC₅₀ Value |
|---|---|
| Type 39 | 25526 ± 5857 |

The above evaluation results show that the HPV39 L1: 59C virus-like particles prepared by the present invention have good immunogenicity, can produce high titers of neutralizing antibodies in animals, and can be used to prepare into a vaccine for preventing HPV infection.

### Example 6.8: Immunogenicity Evaluation of HPV45 L1: 33C Virus-Like Particles in Animals

The experimental methods and procedures were the same as in Example 6.1. The L1 sequence was from Uniprot P36741 and the L2 sequence was from Uniprot P36761.

The results of HPV45 serum pseudovirus neutralization titer assay are detailed in Table 11.

TABLE 11
EC50 of Serum Neutralization Titer Assay in Mice (GMT ± SEM)

| HPV Type | EC₅₀ Value |
|---|---|
| Type 45 | 755 ± 935 |

The above evaluation results show that the HPV45 L1: 33C virus-like particles prepared by the present invention have good immunogenicity, can produce high titers of neutralizing antibodies in animals, and can be used to prepare into a vaccine for preventing HPV infection.

### Example 6.9: Evaluation of Immunogenicity of HPV51 L1: 33C Virus-Like Particles in Animals

The experimental methods and procedures were the same as in Example 6.1. The L1 sequence was from Uniprot P26536 and the L2 sequence was from Uniprot P26539.

The results of HPV51 serum pseudovirus neutralization titer assay are detailed in Table 12.

TABLE 12
EC50 of Serum Neutralization Titer Assay in Mice (GMT ± SEM)

| HPV Type | EC₅₀ Value |
|---|---|
| Type 51 | 5528 ± 1572 |

The above evaluation results show that the HPV51 L1: 33C virus-like particles prepared by the present invention have good immunogenicity, can produce high titers of neutralizing antibodies in animals, and can be used to prepare into a vaccine for preventing HPV infection.

### Example 6.10: Evaluation of Immunogenicity of HPV52 L1: 33C Virus-Like Particles in Animals

The experimental methods and procedures were the same as in Example 6.1. The L1 sequence was from Uniprot Q05138 and the L2 sequence was from Uniprot F8S4U2.

The results of HPV52 serum pseudovirus neutralization titer assay are detailed in Table 13.

TABLE 13
EC50 of Serum Neutralization Titer Assay in Mice (GMT ± SEM)

| HPV Type | EC₅₀ Value |
|---|---|
| Type 52 | 19019 ± 8604 |

The above evaluation results show that the HPV52 L1: 33C virus-like particles prepared by the present invention have good immunogenicity, can produce high titers of neutralizing antibodies in animals, and can be used to prepare into a vaccine for preventing HPV infection.

### Example 6.11: Evaluation of Immunogenicity of HPV56 L1: 33C Virus-Like Particles in Animals

The experimental methods and procedures were the same as in Example 6.1. The L1 sequence was from Uniprot P36743 and the L2 sequence was from Uniprot P36765.

The results of HPV56 serum pseudovirus neutralization titer assay are detailed in Table 14.

TABLE 14
EC50 of Serum Neutralization Titer Assay in Mice (GMT ± SEM)

| HPV Type | EC₅₀ Value |
|---|---|
| Type 56 | 2497 ± 612 |

The above evaluation results show that the HPV56 L1: 33C virus-like particles prepared by the present invention have good immunogenicity, can produce high titers of neutralizing antibodies in animals, and can be used to prepare into a vaccine for preventing HPV infection.

### Example 6.12: Evaluation of Immunogenicity of HPV58 L1: 33C Virus-Like Particles in Animals

The experimental methods and procedures were the same as in Example 6.1. The L1 sequence was from Uniprot P26535 and the L2 sequence was from Uniprot B6ZB12.

The results of HPV58 serum pseudovirus neutralization titer assay are detailed in Table 15.

TABLE 15
EC50 of Serum Neutralization Titer Assay in Mice (GMT ± SEM)

| HPV Type | EC₅₀ Value |
|---|---|
| Type 58 | 19939 ± 8459 |

The above evaluation results show that the HPV58 L1: 33C virus-like particles prepared by the present invention have good immunogenicity, can produce high titers of neutralizing antibodies in animals, and can be used to prepare into a vaccine for the prevention of HPV infection.

### Comparative Example 1: Expression of C-Terminal Truncated HPV16L1 (Aa 1-474)

The inventors attempted to truncate the C-terminus of HPV16L1 by 31 amino acids and named it HPV16L1 (1-474) (SEQ ID NO: 27). However, it was found in the study that the truncated HPV16L1 (1-474) protein was highly expressed but has very poor solubility, and is difficult to extract and purify, the detailed results of expression and extraction are shown in FIG. 3.

Although the present invention has been described in detail by way of illustration and embodiments in the foregoing, it is intended to facilitate understanding. It is obvious to those skilled in the art that various modifications and improvements can be made to the technical solutions of the present invention are apparently possible for those of ordinary skill in the art without deviating from the spirit or scope of the attached claims.

What is claimed is:

1. A chimeric human papillomavirus L1 protein comprising, from its N-terminus to C-terminus orientation,
(a) an N-terminal fragment derived from an L1 protein of HPV Type 31, wherein said fragment is encoded by the gene in SEQ ID No: 57; and
(b) a C-terminal fragment, which is selected from the sequences shown in SEQ ID No: 2 or SEQ ID No: 132,
wherein said chimeric human papillomavirus L1 protein has the immunogenicity of the L1 protein of the HPV type 31.

2. The chimeric human papillomavirus L1 protein according to claim 1, wherein the sequence of the N-terminal fragment derived from
HPV type 31 L1 protein is shown in SEQ ID No: 53.

3. The chimeric human papillomavirus L1 protein according to claim 1, wherein said C-terminus of said N-terminal fragment is connected to the N-terminus of said C-terminal fragment directly or via a linker.

4. The chimeric human papillomavirus L1 protein according to claim 1, wherein the sequence of the chimeric HPV L1 is shown in SEQ ID No: 55.

5. An immunogenic composition for the prevention of papillomavirus-associated diseases, comprising:
(a) an adjuvant; and
(b) the chimeric human papillomavirus L1 protein according to claim 1.

6. An immunogenic composition for the prevention of papillomavirus-associated diseases, comprising:
(a) an adjuvant; and
(b) the chimeric human papillomavirus L1 protein according to claim 2.

7. An immunogenic composition for the prevention of papillomavirus-associated diseases, comprising:
(a) an adjuvant; and
(b) the chimeric human papillomavirus L1 protein according to claim 3.

8. An immunogenic composition for the prevention of papillomavirus-associated diseases, comprising:
(a) an adjuvant; and
(b) the chimeric human papillomavirus L1 protein according to claim 4.


SEQUENCE LISTING (202 sequences)

SEQ ID NO: 1 - protein, 469 aa, Homo sapiens
  REGION 1..469: note = misc_feature - 0601 Amino acid sequence of HPV type 6 L1 protein aa 1-469
MWRPSDSTVYVPPPNPVSKVVATDAYVTRTNIFYHASSSRLLAVGHPYFSIKRANKTVVPKVSGYQYRVFKVVLPDPNKFALPDSSLFDPTTQRLVWACTGLEVGRGQPLGVGVSGHPFLNKYDDVENSGSGGNPGQDNRVNVGMDYKQTQLCMVGCAPPLGEHWGKGKQCTNTPVQAGDCPPLELITSVIQDGDMVDTGFGAMNFADLQTNKSDVPIDICGTTCKYPDYLQMAADPYGDRLFFFLRKEQMFARHFFNRAGEVGEPVPDTLIIKGSGNRTSVGSSIYVNTPSGSLVSSEAQLFNKPYWLQKAQGHNNGICWGNQLFVTVVDTTRSTNMTLCASVTTSSTYTNSDYKEYMRHVEEYDLQFIFQLCSITLSAEVMAYIHTMNPSVLEDWNFGLSPPPNGTLEDTYRYVQSQAITCQKPTPEKEKPDPYKNLSFWEVNLKEKFSSELDQYPLGRKFLLQSGY

SEQ ID NO: 2 - protein, 26 aa, Homo sapiens
  REGION 1..26: note = misc_feature - 0602 Amino acid sequence of HPV type 33 L1 protein aa 474-499
KAKPKLKRAAPTSTRTSSAKRKKVKK

SEQ ID NO: 3 - protein, 495 aa, synthetic construct
  REGION 1..495: note = 0603 Amino acid sequence of chimeric HPV type 6 L1 protein
MWRPSDSTVYVPPPNPVSKVVATDAYVTRTNIFYHASSSRLLAVGHPYFSIKRANKTVVPKVSGYQYRVFKVVLPDPNKFALPDSSLFDPTTQRLVWACTGLEVGRGQPLGVGVSGHPFLNKYDDVENSGSGGNPGQDNRVNVGMDYKQTQLCMVGCAPPLGEHWGKGKQCTNTPVQAGDCPPLELITSVIQDGDMVDTGFGAMNFADLQTNKSDVPIDICGTTCKYPDYLQMAADPYGDRLFFFLRKEQMFARHFFNRAGEVGEPVPDTLIIKGSGNRTSVGSSIYVNTPSGSLVSSEAQLFNKPYWLQKAQGHNNGICWGNQLFVTVVDTTRSTNMTLCASVTTSSTYTNSDYKEYMRHVEEYDLQFIFQLCSITLSAEVMAYIHTMNPSVLEDWNFGLSPPPNGTLEDTYRYVQSQAITCQKPTPEKEKPDPYKNLSFWEVNLKEKFSSELDQYPLGRKFLLQSGYKAKPKLKRAAPTSTRTSSAKRKKVKK

SEQ ID NO: 4 - DNA, 1488 bp, synthetic construct
  misc_feature 1..1488: note = 0604 Nucleotide sequence of chimeric HPV type 6 L1 protein
  source 1..1488: mol_type = other DNA
atgtggagaccatctgacagcacagtctatgtgcctcctccaaaccctgtgagcaaggtggtggctacagatgcctatgtgaccaggaccaacatcttctaccatgcctcctccagcagactgctggctgtgggacacccatacttcagcatcaagagggctaacaagacagtggtgccaaaggtgtctggctaccaatacagggtgttcaaggtggtgctgcctgacccaaacaagtttgccctgcctgactcctccctgtttgacccaaccacccagagactggtgtgggcttgtactggattggaggtgggcaggggacaaccactgggagtgggagtgtctggacacccattcctgaacaaatatgatgatgtggagaactctggctctggaggcaaccctggacaagacaacagggtgaatgtggggatggactacaagcagacccaactttgtatggtgggctgtgcccctccactgggagaacactggggcaagggcaagcagtgtaccaacacacctgtccaggctggagactgtcctccattggaactgattacctctgtgattcaggatggagatatggtggacacaggctttggagctatgaactttgctgacctccaaaccaacaagtctgatgtgccaattgacatctgtggcaccacttgtaaataccctgactacctccaaatggctgctgacccatatggagacagactgttcttcttcctgaggaaggaacagatgtttgccagacacttcttcaacagggctggagaggtgggagaacctgtgcctgacaccctgattatcaagggctctggcaacaggacctctgtgggctccagcatctatgtgaacacaccatctggctccctggtgtcctctgaggctcaacttttcaacaagccatactggctccaaaaggctcaaggacacaacaatggcatctgttggggcaaccaactttttgtgacagtggtggacaccaccaggagcaccaatatgaccctgtgtgcctctgtgaccacctccagcacctacaccaactctgactacaaggaatatatgaggcatgtggaggaatatgacctccaattcatcttccaactttgtagcatcaccctgtctgctgaggtgatggcttacatccacacaatgaacccatctgtgttggaggactggaactttggactgagccctcctccaaatggcaccttggaggacacctacagatatgtccagagccaggctatcacttgtcagaagccaacacctgagaaggagaagcctgacccatacaagaacctgtccttctgggaggtgaacctgaaagagaagttctcctctgaactggaccaatacccactgggcaggaagttcctgctccaatctggctacaaagccaagccaaaactgaaaagggctgccccaaccagcaccaggacctcctctgccaagaggaagaaggtgaagaagtaa

SEQ ID NO: 5 - DNA, 1522 bp, synthetic construct
  misc_feature 1..1522: note = 0605 Synthetic HPV6 L1 gene
  source 1..1522: mol_type = other DNA
ctgggtaccatgtggagaccatctgacagcacagtctatgtgcctcctccaaaccctgtgagcaaggtggtggctacagatgcctatgtgaccaggaccaacatcttctaccatgcctcctccagcagactgctggctgtgggacacccatacttcagcatcaagagggctaacaagacagtggtgccaaaggtgtctggctaccaatacagggtgttcaaggtggtgctgcctgacccaaacaagtttgccctgcctgactcctccctgtttgacccaaccacccagagactggtgtgggcttgtactggattggaggtgggcaggggacaaccactgggagtgggagtgtctggacacccattcctgaacaaatatgatgatgtggagaactctggctctggaggcaaccctggacaagacaacagggtgaatgtggggatggactacaagcagacccaactttgtatggtgggctgtgcccctccactgggagaacactggggcaagggcaagcagtgtaccaacacacctgtccaggctggagactgtcctccattggaactgattacctctgtgattcaggatggagatatggtggacacaggctttggagctatgaactttgctgacctccaaaccaacaagtctgatgtgccaattgacatctgtggcaccacttgtaaataccctgactacctccaaatggctgctgacccatatggagacagactgttcttcttcctgaggaaggaacagatgtttgccagacacttcttcaacagggctggagaggtgggagaacctgtgcctgacaccctgattatcaagggctctggcaacaggacctctgtgggctccagcatctatgtgaacacaccatctggctccctggtgtcctctgaggctcaacttttcaacaagccatactggctccaaaaggctcaaggacacaacaatggcatctgttggggcaaccaactttttgtgacagtggtggacaccaccaggagcaccaatatgaccctgtgtgcctctgtgaccacctccagcacctacaccaactctgactacaaggaatatatgaggcatgtggaggaatatgacctccaattcatcttccaactttgtagcatcaccctgtctgctgaggtgatggcttacatccacacaatgaacccatctgtgttggaggactggaactttggactgagccctcctccaaatggcaccttggaggacacctacagatatgtccagagccaggctatcacttgtcagaagccaacacctgagaaggagaagcctgacccatacaagaacctgtccttctgggaggtgaacctgaaagagaagttctcctctgaactggaccaatacccactgggcaggaagttcctgctccaatctggctacaggggcaggtccagcatcaggacaggagtgaagagacctgctgtgagcaaggcatctgctgccccaaagaggaagagggctaagaccaagaggtaaactcgagctc

SEQ ID NO: 6 - DNA, 1519 bp, synthetic construct
  misc_feature 1..1519: note = 0606 Synthetic HPV33 L1 gene
  source 1..1519: mol_type = other DNA
ctgggtaccatgagtgtgtggagaccatctgaggctacagtctacctgcctcctgtgcctgtgagcaaggtggtgagcacagatgaatatgtgagcaggaccagcatctactactatgctggctccagcagactgctggctgtgggacacccatacttcagcatcaagaacccaaccaatgccaagaaactgctggtgccaaaggtgtctggactccaatacagggtgttcagggtgagactgcctgacccaaacaagtttggctttcctgacacctccttctacaaccctgacacccagagactggtgtgggcttgtgtgggattggagattggcaggggacaaccactgggagtgggcatctctggacacccactgctgaacaagtttgatgacacagagacaggcaacaaataccctggacaacctggagcagacaacagggagtgtctgagtatggactacaagcagacccaactttgtctgctgggctgtaagcctccaacaggagaacactggggcaagggagtggcttgtaccaatgctgcccctgccaatgactgtcctccattggaactgataaacaccatcattgaggatggagatatggtggacacaggctttggctgtatggacttcaagaccctccaagccaacaagtctgatgtgccaattgacatctgtggcagcacttgtaaataccctgactacctgaaaatgacctctgaaccatatggagactccctgttcttcttcctgaggagggaacagatgtttgtgagacacttcttcaacagggctggcaccctgggagaggctgtgcctgatgacctctacatcaagggctctggcaccacagccagcatccagtcctctgccttctttccaacaccatctggcagtatggtgacctctgagagccaacttttcaacaagccatactggctccaaagggctcaaggacacaacaatggcatctgttggggcaaccaggtgtttgtgacagtggtggacaccaccaggagcaccaatatgaccctgtgtacccaggtgacctctgacagcacctacaagaatgagaacttcaaggaatacatcaggcatgtggaggaatatgacctccaatttgtgttccaactttgtaaggtgaccctgacagcagaggtgatgacctacatccatgctatgaaccctgacatcttggaggactggcagtttggactgacacctcctccatctgcctccctccaagacacctacaggtttgtgaccagccaggctatcacttgtcagaagacagtgcctccaaaggagaaggaggacccactgggcaaatacaccttctgggaggtggacctgaaagagaagttctctgctgacctggaccagtttccactgggcaggaagttcctgctccaagcaggactgaaagccaagccaaaactgaaaagggctgccccaaccagcaccaggacctcctctgccaagaggaagaaggtgaagaagtaaactcgagctc

SEQ ID NO: 7 - DNA, 35 bp, synthetic construct
  misc_feature 1..35: note = 0607 HPV6L1 F1
  source 1..35: mol_type = other DNA
cttggtaccatgtggagaccatctgacagcacagt

SEQ ID NO: 8 - DNA, 36 bp, synthetic construct
  misc_feature 1..36: note = 0608 HPV6L1 R1
  source 1..36: mol_type = other DNA
gcttggctttgtagccagattggagcaggaacttcc

SEQ ID NO: 9 - DNA, 1426 bp, synthetic construct
  misc_feature 1..1426: note = 0609 HPV6L1 amplified sequence 1
  source 1..1426: mol_type = other DNA
cttggtaccatgtggagaccatctgacagcacagtctatgtgcctcctccaaaccctgtgagcaaggtggtggctacagatgcctatgtgaccaggaccaacatcttctaccatgcctcctccagcagactgctggctgtgggacacccatacttcagcatcaagagggctaacaagacagtggtgccaaaggtgtctggctaccaatacagggtgttcaaggtggtgctgcctgacccaaacaagtttgccctgcctgactcctccctgtttgacccaaccacccagagactggtgtgggcttgtactggattggaggtgggcaggggacaaccactgggagtgggagtgtctggacacccattcctgaacaaatatgatgatgtggagaactctggctctggaggcaaccctggacaagacaacagggtgaatgtggggatggactacaagcagacccaactttgtatggtgggctgtgcccctccactgggagaacactggggcaagggcaagcagtgtaccaacacacctgtccaggctggagactgtcctccattggaactgattacctctgtgattcaggatggagatatggtggacacaggctttggagctatgaactttgctgacctccaaaccaacaagtctgatgtgccaattgacatctgtggcaccacttgtaaataccctgactacctccaaatggctgctgacccatatggagacagactgttcttcttcctgaggaaggaacagatgtttgccagacacttcttcaacagggctggagaggtgggagaacctgtgcctgacaccctgattatcaagggctctggcaacaggacctctgtgggctccagcatctatgtgaacacaccatctggctccctggtgtcctctgaggctcaacttttcaacaagccatactggctccaaaaggctcaaggacacaacaatggcatctgttggggcaaccaactttttgtgacagtggtggacaccaccaggagcaccaatatgaccctgtgtgcctctgtgaccacctccagcacctacaccaactctgactacaaggaatatatgaggcatgtggaggaatatgacctccaattcatcttccaactttgtagcatcaccctgtctgctgaggtgatggcttacatccacacaatgaacccatctgtgttggaggactggaactttggactgagccctcctccaaatggcaccttggaggacacctacagatatgtccagagccaggctatcacttgtcagaagccaacacctgagaaggagaagcctgacccatacaagaacctgtccttctgggaggtgaacctgaaagagaagttctcctctgaactggaccaatacccactgggcaggaagttcctgctccaatctggctacaaagccaagc

SEQ ID NO: 10 - DNA, 35 bp, synthetic construct
  misc_feature 1..35: note = 0610 HPV6L1 F2
  source 1..35: mol_type = other DNA
atctggctacaaagccaagccaaaactgaaaaggg

SEQ ID NO: 11 - DNA, 37 bp, synthetic construct
  misc_feature 1..37: note = 0611 HPV6L1 R2
  source 1..37: mol_type = other DNA
ctgtctagatttacttcttcaccttcttcctcttggc

SEQ ID NO: 12 - DNA, 101 bp, synthetic construct
  misc_feature 1..101: note = 0612 HPV6L1 amplified sequence 2
  source 1..101: mol_type = other DNA
atctggctacaaagccaagccaaaactgaaaagggctgccccaaccagcaccaggacctcctctgccaagaggaagaaggtgaagaagtaaatctagacag

SEQ ID NO: 13 - protein, 38 aa, Homo sapiens
  REGION 1..38: note = misc_feature - 0613 Amino acid sequence of HPV type 59 L1 protein aa 471-508
LQLGARPKPTIGPRKRAAPAPTSTPSPKRVKRRKSSRK

SEQ ID NO: 14 - protein, 470 aa, Homo sapiens
  REGION 1..470: note = misc_feature - 1101 Amino acid sequence of HPV type 11 L1 protein aa 1-470
MWRPSDSTVYVPPPNPVSKVVATDAYVKRTNIFYHASSSRLLAVGHPYYSIKKVNKTVVPKVSGYQYRVFKVVLPDPNKFALPDSSLFDPTTQRLVWACTGLEVGRGQPLGVGVSGHPLLNKYDDVENSGGYGGNPGQDNRVNVGMDYKQTQLCMVGCAPPLGEHWGKGTQCSNTSVQNGDCPPLELITSVIQDGDMVDTGFGAMNFADLQTNKSDVPLDICGTVCKYPDYLQMAADPYGDRLFFYLRKEQMFARHFFNRAGTVGEPVPDDLLVKGGNNRSSVASSIYVHTPSGSLVSSEAQLFNKPYWLQKAQGHNNGICWGNHLFVTVVDTTRSTNMTLCASVSKSATYTNSDYKEYMRHVEEFDLQFIFQLCSITLSAEVMAYIHTMNPSVLEDWNFGLSPPPNGTLEDTYRYVQSQAITCQKPTPEKEKQDPYKDMSFWEVNLKEKFSSELDQFPLGRKFLLQSGY

SEQ ID NO: 15 - protein, 26 aa, Homo sapiens
  REGION 1..26: note = misc_feature - 1102 Amino acid sequence of HPV type 33 L1 protein aa 474-499
KAKPKLKRAAPTSTRTSSAKRKKVKK

SEQ ID NO: 16 - protein, 496 aa, synthetic construct
  REGION 1..496: note = 1103 Amino acid sequence of chimeric HPV type 11 L1 protein
MWRPSDSTVYVPPPNPVSKVVATDAYVKRTNIFYHASSSRLLAVGHPYYSIKKVNKTVVPKVSGYQYRVFKVVLPDPNKFALPDSSLFDPTTQRLVWACTGLEVGRGQPLGVGVSGHPLLNKYDDVENSGGYGGNPGQDNRVNVGMDYKQTQLCMVGCAPPLGEHWGKGTQCSNTSVQNGDCPPLELITSVIQDGDMVDTGFGAMNFADLQTNKSDVPLDICGTVCKYPDYLQMAADPYGDRLFFYLRKEQMFARHFFNRAGTVGEPVPDDLLVKGGNNRSSVASSIYVHTPSGSLVSSEAQLFNKPYWLQKAQGHNNGICWGNHLFVTVVDTTRSTNMTLCASVSKSATYTNSDYKEYMRHVEEFDLQFIFQLCSITLSAEVMAYIHTMNPSVLEDWNFGLSPPPNGTLEDTYRYVQSQAITCQKPTPEKEKQDPYKDMSFWEVNLKEKFSSELDQFPLGRKFLLQSGYKAKPKLKRAAPTSTRTSSAKRKKVKK

SEQ ID NO: 17 - DNA, 1492 bp, synthetic construct
  misc_feature 1..1492: note = 1104 Nucleotide sequence of chimeric HPV type 11 L1 protein
  source 1..1492: mol_type = other DNA
atgtggagaccatctgacagcacagtctatgtgcctcctccaaaccctgtgagcaaggtggtggctacagatgcctatgtgaagaggaccaacatcttctaccatgcctcctccagcagactgctggctgtgggacacccatactacagcatcaagaaggtgaacaagacagtggtgccaaaggtgtctggctaccaatacagggtgttcaaggtggtgctgcctgacccaaacaagtttgccctgcctgactcctccctgtttgacccaaccacccagagactggtgtgggcttgtactggattggaggtgggcaggggacaaccactgggagtgggagtgtctggacacccactgctgaacaaatatgatgatgtggagaactctggaggctatggaggcaaccctggacaagacaacagggtgaatgtggggatggactacaagcagacccaactttgtatggtgggctgtgcccctccactgggagaacactggggcaagggcacccagtgtagcaacacctctgtccagaatggagactgtcctccattggaactgattacctctgtgattcaggatggagatatggtggacacaggctttggagctatgaactttgctgacctccaaaccaacaagtctgatgtgccactggacatctgtggcacagtgtgtaaataccctgactacctccaaatggctgctgacccatatggagacagactgttcttctacctgaggaaggaacagatgtttgccagacacttcttcaacagggctggcacagtgggagaacctgtgcctgatgacctgctggtgaagggaggcaacaacaggtcctctgtggcatccagcatctatgtgcatacaccatctggctccctggtgtcctctgaggctcaacttttcaacaagccatactggctccaaaaggctcaaggacacaacaatggcatctgttggggcaaccacctgtttgtgacagtggtggacaccaccaggagcaccaatatgaccctgtgtgcctctgtgagcaagtctgccacctacaccaactctgactacaaggaatatatgaggcatgtggaggagtttgacctccaattcatcttccaactttgtagcatcaccctgtctgctgaggtgatggcttacatccacacaatgaacccatctgtgttggaggactggaactttggactgagccctcctccaaatggcaccttggaggacacctacagatatgtccagagccaggctatcacttgtcagaagccaacacctgagaaggagaagcaggacccatacaaggatatgagtttctgggaggtgaacctgaaagagaagttctcctctgaactggaccagtttccactgggcaggaagttcctgctccaatctggctacaaagccaagccaaaactgaaaagggctgccccaaccagcaccaggacctcctctgccaagaggaagaaggtgaagaagtaaa

SEQ ID NO: 18 - DNA, 1525 bp, synthetic construct
  misc_feature 1..1525: note = 1105 Synthetic HPV11 L1 gene
  source 1..1525: mol_type = other DNA
ctgggtaccatgtggagaccatctgacagcacagtctatgtgcctcctccaaaccctgtgagcaaggtggtggctacagatgcctatgtgaagaggaccaacatcttctaccatgcctcctccagcagactgctggctgtgggacacccatactacagcatcaagaaggtgaacaagacagtggtgccaaaggtgtctggctaccaatacagggtgttcaaggtggtgctgcctgacccaaacaagtttgccctgcctgactcctccctgtttgacccaaccacccagagactggtgtgggcttgtactggattggaggtgggcaggggacaaccactgggagtgggagtgtctggacacccactgctgaacaaatatgatgatgtggagaactctggaggctatggaggcaaccctggacaagacaacagggtgaatgtggggatggactacaagcagacccaactttgtatggtgggctgtgcccctccactgggagaacactggggcaagggcacccagtgtagcaacacctctgtccagaatggagactgtcctccattggaactgattacctctgtgattcaggatggagatatggtggacacaggctttggagctatgaactttgctgacctccaaaccaacaagtctgatgtgccactggacatctgtggcacagtgtgtaaataccctgactacctccaaatggctgctgacccatatggagacagactgttcttctacctgaggaaggaacagatgtttgccagacacttcttcaacagggctggcacagtgggagaacctgtgcctgatgacctgctggtgaagggaggcaacaacaggtcctctgtggcatccagcatctatgtgcatacaccatctggctccctggtgtcctctgaggctcaacttttcaacaagccatactggctccaaaaggctcaaggacacaacaatggcatctgttggggcaaccacctgtttgtgacagtggtggacaccaccaggagcaccaatatgaccctgtgtgcctctgtgagcaagtctgccacctacaccaactctgactacaaggaatatatgaggcatgtggaggagtttgacctccaattcatcttccaactttgtagcatcaccctgtctgctgaggtgatggcttacatccacacaatgaacccatctgtgttggaggactggaactttggactgagccctcctccaaatggcaccttggaggacacctacagatatgtccagagccaggctatcacttgtcagaagccaacacctgagaaggagaagcaggacccatacaaggatatgagtttctgggaggtgaacctgaaagagaagttctcctctgaactggaccagtttccactgggcaggaagttcctgctccaatctggctacaggggcaggacctctgccaggacaggcatcaagagacctgctgtgagcaagccaagcacagccccaaagaggaagaggaccaagaccaagaagtaaactcgagctc

SEQ ID NO: 19 - DNA, 1519 bp, synthetic construct; identical to SEQ ID NO: 6
  misc_feature 1..1519: note = 1106 Synthetic HPV33 L1 gene
  source 1..1519: mol_type = other DNA

SEQ ID NO: 20 - DNA, 35 bp, synthetic construct; identical to SEQ ID NO: 7
  misc_feature 1..35: note = 1107 HPV11L1 F1
  source 1..35: mol_type = other DNA

SEQ ID NO: 21 - DNA, 36 bp, synthetic construct; identical to SEQ ID NO: 8
  misc_feature 1..36: note = 1108 HPV11L1 R1
  source 1..36: mol_type = other DNA

SEQ ID NO: 22 - DNA, 1429 bp, synthetic construct
  misc_feature 1..1429: note = 1109 HPV11L1 amplified sequence 1
  source 1..1429: mol_type = other DNA
cttggtaccatgtggagaccatctgacagcacagtctatgtgcctcctccaaaccctgtgagcaaggtggtggctacagatgcctatgtgaagaggaccaacatcttctaccatgcctcctccagcagactgctggctgtgggacacccatactacagcatcaagaaggtgaacaagacagtggtgccaaaggtgtctggctaccaatacagggtgttcaaggtggtgctgcctgacccaaacaagtttgccctgcctgactcctccctgtttgacccaaccacccagagactggtgtgggcttgtactggattggaggtgggcaggggacaaccactgggagtgggagtgtctggacacccactgctgaacaaatatgatgatgtggagaactctggaggctatggaggcaaccctggacaagacaacagggtgaatgtggggatggactacaagcagacccaactttgtatggtgggctgtgcccctccactgggagaacactggggcaagggcacccagtgtagcaacacctctgtccagaatggagactgtcctccattggaactgattacctctgtgattcaggatggagatatggtggacacaggctttggagctatgaactttgctgacctccaaaccaacaagtctgatgtgccactggacatctgtggcacagtgtgtaaataccctgactacctccaaatggctgctgacccatatggagacagactgttcttctacctgaggaaggaacagatgtttgccagacacttcttcaacagggctggcacagtgggagaacctgtgcctgatgacctgctggtgaagggaggcaacaacaggtcctctgtggcatccagcatctatgtgcatacaccatctggctccctggtgtcctctgaggctcaacttttcaacaagccatactggctccaaaaggctcaaggacacaacaatggcatctgttggggcaaccacctgtttgtgacagtggtggacaccaccaggagcaccaatatgaccctgtgtgcctctgtgagcaagtctgccacctacaccaactctgactacaaggaatatatgaggcatgtggaggagtttgacctccaattcatcttccaactttgtagcatcaccctgtctgctgaggtgatggcttacatccacacaatgaacccatctgtgttggaggactggaactttggactgagccctcctccaaatggcaccttggaggacacctacagatatgtccagagccaggctatcacttgtcagaagccaacacctgagaaggagaagcaggacccatacaaggatatgagtttctgggaggtgaacctgaaagagaagttctcctctgaactggaccagtttccactgggcaggaagttcctgctccaatctggctacaaagccaagc

SEQ ID NO: 23 - DNA, 35 bp, synthetic construct; identical to SEQ ID NO: 10
  misc_feature 1..35: note = 1110 HPV11L1 F2
  source 1..35: mol_type = other DNA

SEQ ID NO: 24 - DNA, 37 bp, synthetic construct; identical to SEQ ID NO: 11
  misc_feature 1..37: note = 1111 HPV11L1 R2
  source 1..37: mol_type = other DNA

SEQ ID NO: 25 - DNA, 101 bp, synthetic construct; identical to SEQ ID NO: 12
  misc_feature 1..101: note = 1112 HPV11L1 amplified sequence 2
  source 1..101: mol_type = other DNA

SEQ ID NO: 26 - protein, 38 aa, Homo sapiens; identical to SEQ ID NO: 13
  REGION 1..38: note = misc_feature - 1113 Amino acid sequence of HPV type 59 L1 protein aa 471-508

SEQ ID NO: 27 - protein, 474 aa, Homo sapiens
  REGION 1..474: note = misc_feature - 1601 Amino acid sequence of HPV type 16 L1 protein aa 1-474
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGL

SEQ ID NO: 28 - protein, 26 aa, Homo sapiens
  REGION 1..26: note = misc_feature - 1602 Amino acid sequence of HPV type 33 L1 protein aa 474-499
KAKPKLKRAAPTSTRTSSAKRKKVKK

SEQ ID NO: 29 - protein, 500 aa, synthetic construct
  REGION 1..500: note = 1603 Amino acid sequence of chimeric HPV type 16 L1 protein
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKLKRAAPTSTRTSSAKRKKVKK

SEQ ID NO: 30 - DNA, 1504 bp, synthetic construct
  misc_feature 1..1504: note = 1604 Nucleotide sequence of chimeric HPV type 16 L1 protein
  source 1..1504: mol_type = other DNA
atgagtctgtggctgccatctgaggctacagtctacctgcctcctgtgcctgtgagcaaggtggtgagcacagatgaatatgtggcaaggaccaacatctactaccatgctggcaccagcagactgctggctgtgggacacccatactttccaatcaagaagccaaacaacaacaagattctggtgccaaaggtgtctggactccaatacagggtgttcaggattcacctgcctgacccaaacaagtttggctttcctgacacctccttctacaaccctgacacccagagactggtgtgggcttgtgtgggagtggaggtgggcaggggacaaccactgggagtgggcatctctggacacccactgctgaacaaactggatgacacagagaatgcctctgcctatgctgccaatgctggagtggacaacagggagtgtatcagtatggactacaagcagacccaactttgtctgattggctgtaagcctccaattggagaacactggggcaagggcagcccatgtaccaatgtggctgtgaaccctggagactgtcctccattggaactgataaacacagtgattcaggatggagatatggtggacacaggctttggagctatggacttcaccaccctccaagccaacaagtctgaggtgccactggacatctgtaccagcatctgtaaataccctgactacatcaagatggtgtctgaaccatatggagactccctgttcttctacctgaggagggaacagatgtttgtgagacacctgttcaacagggctggagcagtgggagagaatgtgcctgatgacctctacatcaagggctctggcagcacagccaacctggcatccagcaactactttccaacaccatctggcagtatggtgacctctgatgcccagattttcaacaagccatactggctccaaagggctcaaggacacaacaatggcatctgttggggcaaccaactttttgtgacagtggtggacaccaccaggagcaccaatatgagtctgtgtgctgccatcagcacctctgagaccacctacaagaacaccaacttcaaggaatacctgagacatggagaggaatatgacctccaattcatcttccaactttgtaagattaccctgacagcagatgtgatgacctacatccacagtatgaacagcaccatcttggaggactggaactttggactccaacctcctcctggaggcaccttggaggacacctacaggtttgtgaccagccaggctattgcctgtcagaaacacacacctcctgccccaaaggaggacccactgaaaaaatacaccttctgggaggtgaacctgaaagagaagttctctgctgacctggaccagtttccactgggcaggaagttcctgctccaagcaggactgaaagccaagccaaaactgaaaagggctgccccaaccagcaccaggacctcctctgccaagaggaagaaggtgaagaagtaaa

SEQ ID NO: 31 - DNA, 1537 bp, synthetic construct
  misc_feature 1..1537: note = 1605 Synthetic HPV16 L1 gene
  source 1..1537: mol_type = other DNA
ctgggtaccatgagtctgtggctgccatctgaggctacagtctacctgcctcctgtgcctgtgagcaaggtggtgagcacagatgaatatgtggcaaggaccaacatctactaccatgctggcaccagcagactgctggctgtgggacacccatactttccaatcaagaagccaaacaacaacaagattctggtgccaaaggtgtctggactccaatacagggtgttcaggattcacctgcctgacccaaacaagtttggctttcctgacacctccttctacaaccctgacacccagagactggtgtgggcttgtgtgggagtggaggtgggcaggggacaaccactgggagtgggcatctctggacacccactgctgaacaaactggatgacacagagaatgcctctgcctatgctgccaatgctggagtggacaacagggagtgtatcagtatggactacaagcagacccaactttgtctgattggctgtaagcctccaattggagaacactggggcaagggcagcccatgtaccaatgtggctgtgaaccctggagactgtcctccattggaactgataaacacagtgattcaggatggagatatggtggacacaggctttggagctatggacttcaccaccctccaagccaacaagtctgaggtgccactggacatctgtaccagcatctgtaaataccctgactacatcaagatggtgtctgaaccatatggagactccctgttcttctacctgaggagggaacagatgtttgtgagacacctgttcaacagggctggagcagtgggagagaatgtgcctgatgacctctacatcaagggctctggcagcacagccaacctggcatccagcaactactttccaacaccatctggcagtatggtgacctctgatgcccagattttcaacaagccatactggctccaaagggctcaaggacacaacaatggcatctgttggggcaaccaactttttgtgacagtggtggacaccaccaggagcaccaatatgagtctgtgtgctgccatcagcacctctgagaccacctacaagaacaccaacttcaaggaatacctgagacatggagaggaatatgacctccaattcatcttccaactttgtaagattaccctgacagcagatgtgatgacctacatccacagtatgaacagcaccatcttggaggactggaactttggactccaacctcctcctggaggcaccttggaggacacctacaggtttgtgaccagccaggctattgcctgtcagaaacacacacctcctgccccaaaggaggacccactgaaaaaatacaccttctgggaggtgaacctgaaagagaagttctctgctgacctggaccagtttccactgggcaggaagttcctgctccaagcaggactgaaagccaagccaaagttcaccctgggcaagaggaaggctacaccaaccacctccagcaccagcaccacagccaagaggaagaagaggaaactgtaaactcgagctc

SEQ ID NO: 32 - DNA, 1519 bp, synthetic construct; identical to SEQ ID NO: 6
  misc_feature 1..1519: note = 1606 Synthetic HPV33 L1 gene
  source 1..1519: mol_type = other DNA

SEQ ID NO: 33 - DNA, 34 bp, synthetic construct
  misc_feature 1..34: note = 1607 HPV16L1 F1
  source 1..34: mol_type = other DNA
cttggtaccatgagtctgtggctgccatctgagg

SEQ ID NO: 34 - DNA, 36 bp, synthetic construct
  misc_feature 1..36: note = 1608 HPV16L1 R1
  source 1..36: mol_type = other DNA
gcttggctttcagtcctgcttggagcaggaacttcc

SEQ ID NO: 35 - DNA, 1441 bp, synthetic construct
  misc_feature 1..1441: note = 1609 HPV16L1 amplified sequence 1
  source 1..1441: mol_type = other DNA
cttggtaccatgagtctgtggctgccatctgaggctacagtctacctgcctcctgtgcctgtgagcaaggtggtgagcacagatgaatatgtggcaaggaccaacatctactaccatgctggcaccagcagactgctggctgtgggacacccatactttccaatcaagaagccaaacaacaacaagattctggtgccaaaggtgtctggactccaatacagggtgttcaggattcacctgcctgacccaaacaagtttggctttcctgacacctccttctacaaccctgacacccagagactggtgtgggcttgtgtgggagtggaggtgggcaggggacaaccactgggagtgggcatctctggacacccactgctgaacaaactggatgacacagagaatgcctctgcctatgctgccaatgctggagtggacaacagggagtgtatcagtatggactacaagcagacccaactttgtctgattggctgtaagcctccaattggagaacactggggcaagggcagcccatgtaccaatgtggctgtgaaccctggagactgtcctccattggaactgataaacacagtgattcaggatggagatatggtggacacaggctttggagctatggacttcaccaccctccaagccaacaagtctgaggtgccactggacatctgtaccagcatctgtaaataccctgactacatcaagatggtgtctgaaccatatggagactccctgttcttctacctgaggagggaacagatgtttgtgagacacctgttcaacagggctggagcagtgggagagaatgtgcctgatgacctctacatcaagggctctggcagcacagccaacctggcatccagcaactactttccaacaccatctggcagtatggtgacctctgatgcccagattttcaacaagccatactggctccaaagggctcaaggacacaacaatggcatctgttggggcaaccaactttttgtgacagtggtggacaccaccaggagcaccaatatgagtctgtgtgctgccatcagcacctctgagaccacctacaagaacaccaacttcaaggaatacctgagacatggagaggaatatgacctccaattcatcttccaactttgtaagattaccctgacagcagatgtgatgacctacatccacagtatgaacagcaccatcttggaggactggaactttggactccaacctcctcctggaggcaccttggaggacacctacaggtttgtgaccagccaggctattgcctgtcagaaacacacacctcctgccccaaaggaggacccactgaaaaaatacaccttctgggaggtgaacctgaaagagaagttctctgctgacctggaccagtttccactgggcaggaagttcctgctccaagcaggactgaaagccaagc

SEQ ID NO: 36 - DNA, 35 bp, synthetic construct
  misc_feature 1..35: note = 1610 HPV16L1 F2
  source 1..35: mol_type = other DNA
agcaggactgaaagccaagccaaaactgaaaaggg

SEQ ID NO: 37 - DNA, 36 bp, synthetic construct
  misc_feature 1..36: note = 1611 HPV16L1 R2
  source 1..36: mol_type = other DNA
ctgtctagatttacttcttcaccttcttcctcttgg

SEQ ID NO: 38 - DNA, 101 bp, synthetic construct
  misc_feature 1..101: note = 1612 HPV16L1 amplified sequence 2
  source 1..101: mol_type = other DNA
agcaggactgaaagccaagccaaaactgaaaagggctgccccaaccagcaccaggacctcctctgccaagaggaagaaggtgaagaagtaaatctagacag

SEQ ID NO: 39 - protein, 38 aa, Homo sapiens; identical to SEQ ID NO: 13
  REGION 1..38: note = misc_feature - 1613 Amino acid sequence of HPV type 59 L1 protein aa 471-508

SEQ ID NO: 40 - protein, 470 aa, Homo sapiens
  REGION 1..470: note = misc_feature - 1801 Amino acid sequence of HPV type 18 L1 protein aa 1-470
MALWRPSDNTVYLPPPSVARVVNTDDYVTRTSIFYHAGSSRLLTVGNPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFL

SEQ ID NO: 41 - protein, 26 aa, Homo sapiens
  REGION 1..26: note = misc_feature - 1802 Amino acid sequence of HPV type 33 L1 protein aa 474-499
KAKPKLKRAAPTSTRTSSAKRKKVKK

SEQ ID NO: 42 - protein, 496 aa, synthetic construct
  REGION 1..496: note = 1803 Amino acid sequence of chimeric HPV type 18 L1 protein
MALWRPSDNTVYLPPPSVARVVNTDDYVTRTSIFYHAGSSRLLTVGNPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLKAKPKLKRAAPTSTRTSSAKRKKVKK

SEQ ID NO: 43 - DNA, 1492 bp, synthetic construct
  misc_feature 1..1492: note = 1804 Nucleotide sequence of chimeric HPV type 18 L1 protein
  source 1..1492: mol_type = other DNA
atggccctctggagaccatccgataacacagtgtacttgcccccacccagcgtcgcccgggtggtgaacacagacgactacgtcaccagaacctcaatcttctaccacgccgggtccagccggctgctgaccgtgggcaacccctacttccgcgtgcccgccggcggcggaaacaaacaagacatccccaaagtcagcgcctatcagtaccgggtgttccgcgtccaactgcccgatcccaacaagttcggcctgcccgacacctccatctacaaccccgagacccagaggctggtctgggcttgcgccggcgtcgagatcgggaggggccaacccctgggcgtggggttgtccggccaccccttctacaacaagctggacgataccgagtccagccacgcagcaaccagcaacgtctccgaagatgtgcgcgataacgtcagcgtggactacaaacaaacccaactgtgcatcctgggatgcgcacccgccatcggcgagcattgggccaaggggaccgcctgcaagagcaggcccctgagccaaggggactgtccacccctggagttgaagaataccgtgctcgaggacggcgacatggtggacaccggctacggcgctatggatttctccaccctccaggacaccaagtgcgaagtgcccctcgacatctgccaaagcatctgcaagtaccccgactacctccagatgagcgccgacccctacggcgacagcatgttcttctgtctcagaagggaacaattgttcgcccgccacttctggaaccgggccggcacaatgggagatacagtcccccagagcctgtacatcaaggggaccggaatgagggccagccccgggtcctgcgtctacagcccaagcccctccgggagcatcgtcacaagcgatagccaactcttcaacaagccctactggctccacaaagcccaaggccacaataacggggtgtgttggcacaaccagctgttcgtgaccgtcgtggacacaaccaggtccacaaacctgaccatctgcgccagcacccaaagccccgtgcccggccagtacgacgccacaaagttcaaacaatactctcggcacgtggaagagtacgacctccaattcatcttccaactctgcaccatcaccctcaccgccgacgtgatgagctacatccactccatgaactcctccatcctggaagactggaatttcggcgtgccaccaccccctaccacctccctcgtcgacacctacagattcgtgcagagcgtggccatcacatgccagaaagacgccgcccccgccgagaacaaagacccatacgacaaactgaaattctggaacgtcgacctgaaagagaaattcagcctggatctggaccagtacccattgggcaggaagttcctcaaagccaagccaaaactgaaaagggctgccccaaccagcaccaggacctcctctgccaagaggaagaaggtgaagaagtaaa

SEQ ID NO: 44 - DNA, 1543 bp, synthetic construct
  misc_feature 1..1543: note = 1805 Synthetic HPV18 L1 gene
  source 1..1543: mol_type = other DNA
ctgggtaccatggccctctggagaccatccgataacacagtgtacttgcccccacccagcgtcgcccgggtggtgaacacagacgactacgtcaccagaacctcaatcttctaccacgccgggtccagccggctgctgaccgtgggcaacccctacttccgcgtgcccgccggcggcggaaacaaacaagacatccccaaagtcagcgcctatcagtaccgggtgttccgcgtccaactgcccgatcccaacaagttcggcctgcccgacacctccatctacaaccccgagacccagaggctggtctgggcttgcgccggcgtcgagatcgggaggggccaacccctgggcgtggggttgtccggccaccccttctacaacaagctggacgataccgagtccagccacgcagcaaccagcaacgtctccgaagatgtgcgcgataacgtcagcgtggactacaaacaaacccaactgtgcatcctgggatgcgcacccgccatcggcgagcattgggccaaggggaccgcctgcaagagcaggcccctgagccaaggggactgtccacccctggagttgaagaataccgtgctcgaggacggcgacatggtggacaccggctacggcgctatggatttctccaccctccaggacaccaagtgcgaagtgcccctcgacatctgccaaagcatctgcaagtaccccgactacctccagatgagcgccgacccctacggcgacagcatgttcttctgtctcagaagggaacaattgttcgcccgccacttctggaaccgggccggcacaatgggagatacagtcccccagagcctgtacatcaaggggaccggaatgagggccagccccgggtcctgcgtctacagcccaagcccctccgggagcatcgtcacaagcgatagccaactcttcaacaagccctactggctccacaaagcccaaggccacaataacggggtgtgttggcacaaccagctgttcgtgaccgtcgtggacacaaccaggtccacaaacctgaccatctgcgccagcacccaaagccccgtgcccggccagtacgacgccacaaagttcaaacaatactctcggcacgtggaagagtacgacctccaattcatcttccaactctgcaccatcaccctcaccgccgacgtgatgagctacatccactccatgaactcctccatcctggaagactggaatttcggcgtgccaccaccccctaccacctccctcgtcgacacctacagattcgtgcagagcgtggccatcacatgccagaaagacgccgcccccgccgagaacaaagacccatacgacaaactgaaattctggaacgtcgacctgaaagagaaattcagcctggatctggaccagtacccattgggcaggaagttcctcgtgcaagccggcctcaggagaaaaccaacaatcgggcccaggaagaggagcgcccccagcgcaaccaccagcagcaagcccgcaaaaagggtcagagtgagggcacgcaaataaactcgagctc

SEQ ID NO: 45 - DNA, 1519 bp, synthetic construct; identical to SEQ ID NO: 6
  misc_feature 1..1519: note = 1806 Synthetic HPV33 L1 gene
  source 1..1519: mol_type = other DNA

SEQ ID NO: 46 - DNA, 34 bp, synthetic construct
  misc_feature 1..34: note = 1807 HPV18L1 F1
  source 1..34: mol_type = other DNA
cttggtaccatggccctctggagaccatccgata

SEQ ID NO: 47 - DNA, 35 bp, synthetic construct
  misc_feature 1..35: note = 1808 HPV18L1 R1
  source 1..35: mol_type = other DNA
gcttggctttgaggaacttcctgcccaatgggtac

SEQ ID NO: 48 - DNA, 1429 bp, synthetic construct
  misc_feature 1..1429: note = 1809 HPV18L1 amplified sequence 1
  source 1..1429: mol_type = other DNA
cttggtaccatggccctctggagaccatccgataacacagtgtacttgcccccacccagcgtcgcccgggtggtgaacacagacgactacgtcaccagaacctcaatcttctaccacgccgggtccagccggctgctgaccgtgggcaacccctacttccgcgtgcccgccggcggcggaaacaaacaagacatccccaaagtcagcgcctatcagtaccgggtgttccgcgtccaactgcccgatcccaacaagttcggcctgcccgacacctccatctacaaccccgagacccagaggctggtctgggcttgcgccggcgtcgagatcgggaggggccaacccctgggcgtggggttgtccggccaccccttctacaacaagctggacgataccgagtccagccacgcagcaaccagcaacgtctccgaagatgtgcgcgataacgtcagcgtggactacaaacaaacccaactgtgcatcctgggatgcgcacccgccatcggcgagcattgggccaaggggaccgcctgcaagagcaggcccctgagccaaggggactgtccacccctggagttgaagaataccgtgctcgaggacggcgacatggtggacaccggctacggcgctatggatttctccaccctccaggacaccaagtgcgaagtgcccctcgacatctgccaaagcatctgcaagtaccccgactacctccagatgagcgccgacccctacggcgacagcatgttcttctgtctcagaagggaacaattgttcgcccgccacttctggaaccgggccggcacaatgggagatacagtcccccagagcctgtacatcaaggggaccggaatgagggccagccccgggtcctgcgtctacagcccaagcccctccgggagcatcgtcacaagcgatagccaactcttcaacaagccctactggctccacaaagcccaaggccacaataacggggtgtgttggcacaaccagctgttcgtgaccgtcgtggacacaaccaggtccacaaacctgaccatctgcgccagcacccaaagccccgtgcccggccagtacgacgccacaaagttcaaacaatactctcggcacgtggaagagtacgacctccaattcatcttccaactctgcaccatcaccctcaccgccgacgtgatgagctacatccactccatgaactcctccatcctggaagactggaatttcggcgtgccaccaccccctaccacctccctcgtcgacacctacagattcgtgcagagcgtggccatcacatgccagaaagacgccgcccccgccgagaacaaagacccatacgacaaactgaaattctggaacgtcgacctgaaagagaaattcagcctggatctggaccagtacccattgggcaggaagttcctcaaagccaagc

SEQ ID NO: 49 - DNA, 35 bp, synthetic construct
  misc_feature 1..35: note = 1810 HPV18L1 F2
  source 1..35: mol_type = other DNA
gaagttcctcaaagccaagccaaaactgaaaaggg

SEQ ID NO: 50 - DNA, 36 bp, synthetic construct; identical to SEQ ID NO: 37
  misc_feature 1..36: note = 1811 HPV18L1 R2
  source 1..36: mol_type = other DNA

SEQ ID NO: 51 - DNA, 101 bp, synthetic construct
  misc_feature 1..101: note = 1812 HPV18L1 amplified sequence 2
  source 1..101: mol_type = other DNA
gaagttcctcaaagccaagccaaaactgaaaagggctgccccaaccagcaccaggacctcctctgccaagaggaagaaggtgaagaagtaaatctagacag

SEQ ID NO: 52 - protein, 38 aa, Homo sapiens; identical to SEQ ID NO: 13
  REGION 1..38: note = misc_feature - 1813 Amino acid sequence of HPV type 59 L1 protein aa 471-508

SEQ ID NO: 53 - protein, 475 aa, Homo sapiens
  REGION 1..475: note = misc_feature - 3101 Amino acid sequence of HPV type 31 L1 protein aa 1-475
MSLWRPSEATVYLPPVPVSKVVSTDEYVTRTNIYYHAGSARLLTVGHPYYSIPKSDNPKKIVVPKVSGLQYRVFRVRLPDPNKFGFPDTSFYNPETQRLVWACVGLEVGRGQPLGVGISGHPLLNKFDDTENSNRYAGGPGTDNRECISMDYKQTQLCLLGCKPPIGEHWGKGSPCSNNAITPGDCPPLELKNSVIQDGDMVDTGFGAMDFTALQDTKSNVPLDICNSICKYPDYLKMVAEPYGDTLFFYLRREQMFVRHFFNRSGTVGESVPTDLYIKGSGSTATLANSTYFPTPSGSMVTSDAQIFNKPYWMQRAQGHNNGICWGNQLFVTVVDTTRSTNMSVCAAIANSDTTFKSSNFKEYLRHGEEFDLQFIFQLCKITLSADIMTYIHSMNPAILEDWNFGLTTPPSGSLEDTYRFVTSQAITCQKSAPQKPKEDPFKDYVFWEVNLKEKFSADLDQFPLGRKFLLQAGY

SEQ ID NO: 54 - protein, 26 aa, Homo sapiens
  REGION 1..26: note = misc_feature - 3102 Amino acid sequence of HPV type 33 L1 protein aa 474-499
KAKPKLKRAAPTSTRTSSAKRKKVKK

SEQ ID NO: 55 - protein, 501 aa, synthetic construct
  REGION 1..501: note = 3103 Amino acid sequence of chimeric HPV type 31 L1 protein
MSLWRPSEATVYLPPVPVSKVVSTDEYVTRTNIYYHAGSARLLTVGHPYYSIPKSDNPKKIVVPKVSGLQYRVFRVRLPDPNKFGFPDTSFYNPETQRLVWACVGLEVGRGQPLGVGISGHPLLNKFDDTENSNRYAGGPGTDNRECISMDYKQTQLCLLGCKPPIGEHWGKGSPCSNNAITPGDCPPLELKNSVIQDGDMVDTGFGAMDFTALQDTKSNVPLDICNSICKYPDYLKMVAEPYGDTLFFYLRREQMFVRHFFNRSGTVGESVPTDLYIKGSGSTATLANSTYFPTPSGSMVTSDAQIFNKPYWMQRAQGHNNGICWGNQLFVTVVDTTRSTNMSVCAAIANSDTTFKSSNFKEYLRHGEEFDLQFIFQLCKITLSADIMTYIHSMNPAILEDWNFGLTTPPSGSLEDTYRFVTSQAITCQKSAPQKPKEDPFKDYVFWEVNLKEKFSADLDQFPLGRKFLLQAGYKAKPKLKRAAPTSTRTSSAKRKKVKK

SEQ ID NO: 56 - DNA, 1507 bp, synthetic construct
  misc_feature 1..1507: note = 3104 Nucleotide sequence of chimeric HPV type 31 L1 protein
  source 1..1507: mol_type = other DNA
atgagcctgtggaggcccagcgaggccaccgtgtacctgccccccgtgcccgtgagcaaggtggtgagcaccgacgagtacgtgaccaggaccaacatctactaccacgccggcagcgccaggctgctgaccgtgggccacccctactacagcatccccaagagcgacaaccccaagaagatcgtggtgcccaaggtgagcggcctgcagtacagggtgttcagggtgaggctgcccgaccccaacaagttcggcttccccgacaccagcttctacaaccccgagacccagaggctggtgtgggcctgcgtgggcctggaggtgggcaggggccagcccctgggcgtgggcatcagcggccaccccctgctgaacaagttcgacgacaccgagaacagcaacaggtacgccggcggccccggcaccgacaacagggagtgcatcagcatggactacaagcagacccagctgtgcctgctgggctgcaagccccccatcggcgagcactggggcaagggcagcccctgcagcaacaacgccatcacccccggcgactgcccccccctggagctgaagaacagcgtgatccaggacggcgacatggtggacaccggcttcggcgccatggacttcaccgccctgcaggacaccaagagcaacgtgcccctggacatctgcaacagcatctgcaagtaccccgactacctgaagatggtggccgagccctacggcgacaccctgttcttctacctgaggagggagcagatgttcgtgaggcacttcttcaacaggagcggcaccgtgggcgagagcgtgcccaccgacctgtacatcaagggcagcggcagcaccgccaccctggccaacagcacctacttccccacccccagcggcagcatggtgaccagcgacgcccagatcttcaacaagccctactggatgcagagggcccagggccacaacaacggcatctgctggggcaaccagctgttcgtgaccgtggtggacaccaccaggagcaccaacatgagcgtgtgcgccgccatcgccaacagcgacaccaccttcaagagcagcaacttcaaggagtacctgaggcacggcgaggagttcgacctgcagttcatcttccagctgtgcaagatcaccctgagcgccgacatcatgacctacatccacagcatgaaccccgccatcctggaggactggaacttcggcctgaccaccccccccagcggcagcctggaggacacctacaggttcgtgaccagccaggccatcacctgccagaagtccgccccccagaagcccaaggaggaccccttcaaggactacgtgttctgggaggtgaacctgaaggagaagttcagcgccgacctggaccagttccccctgggcaggaagttcctgctgcaggccggctacaaagccaagccaaaactgaaaagggctgccccaaccagcaccaggacctcctctgccaagaggaagaaggtgaagaagtaaa

SEQ ID NO: 57 - DNA, 1534 bp, synthetic construct
  misc_feature 1..1534: note = 3105 Synthetic HPV31 L1 gene
  source 1..1534: mol_type = other DNA
ctgggtaccatgagcctgtggaggcccagcgaggccaccgtgtacctgccccccgtgcccgtgagcaaggtggtgagcaccgacgagtacgtgaccaggaccaacatctactaccacgccggcagcgccaggctgctgaccgtgggccacccctactacagcatccccaagagcgacaaccccaagaagatcgtggtgcccaaggtgagcggcctgcagtacagggtgttcagggtgaggctgcccgaccccaacaagttcggcttccccgacaccagcttctacaaccccgagacccagaggctggtgtgggcctgcgtgggcctggaggtgggcaggggccagcccctgggcgtgggcatcagcggccaccccctgctgaacaagttcgacgacaccgagaacagcaacaggtacgccggcggccccggcaccgacaacagggagtgcatcagcatggactacaagcagacccagctgtgcctgctgggctgcaagccccccatcggcgagcactggggcaagggcagcccctgcagcaacaacgccatcacccccggcgactgcccccccctggagctgaagaacagcgtgatccaggacggcgacatggtggacaccggcttcggcgccatggacttcaccgccctgcaggacaccaagagcaacgtgcccctggacatctgcaacagcatctgcaagtaccccgactacctgaagatggtggccgagccctacggcgacaccctgttcttctacctgaggagggagcagatgttcgtgaggcacttcttcaacaggagcggcaccgtgggcgagagcgtgcccaccgacctgtacatcaagggcagcggcagcaccgccaccctggccaacagcacctacttccccacccccagcggcagcatggtgaccagcgacgcccagatcttcaacaagccctactggatgcagagggcccagggccacaacaacggcatctgctggggcaaccagctgttcgtgaccgtggtggacaccaccaggagcaccaacatgagcgtgtgcgccgccatcgccaacagcgacaccaccttcaagagcagcaacttcaaggagtacctgaggcacggcgaggagttcgacctgcagttcatcttccagctgtgcaagatcaccctgagcgccgacatcatgacctacatccacagcatgaaccccgccatcctggaggactggaacttcggcctgaccaccccccccagcggcagcctggaggacacctacaggttcgtgaccagccaggccatcacctgccagaagtccgccccccagaagcccaaggaggaccccttcaaggactacgtgttctgggaggtgaacctgaaggagaagttcagcgccgacctggaccagttccccctgggcaggaagttcctgctgcaggccggctacagggccaggcccaagttcaaggccggcaagaggagcgcccccagcgccagcaccaccacccccgccaagaggaagaagaccaagaagtaaactcgagctc

SEQ ID NO: 58 - DNA, 1519 bp, synthetic construct; identical to SEQ ID NO: 6
  misc_feature 1..1519: note = 3106 Synthetic HPV33 L1 gene
  source 1..1519: mol_type = other DNA

SEQ ID NO: 59 - DNA, 33 bp, synthetic construct
  misc_feature 1..33: note = 3107 HPV31L1 F1
  source 1..33: mol_type = other DNA
cttggtaccatgagcctgtggaggcccagcgag

SEQ ID NO: 60 - DNA, 38 bp, synthetic construct
  misc_feature 1..38: note = 3108 HPV31L1 R1
  source 1..38: mol_type = other DNA
gcttggctttgtagccggcctgcagcaggaacttcctg

SEQ ID NO: 61 - DNA, 1444 bp, synthetic construct
  misc_feature 1..1444: note = 3109 HPV31L1 amplified sequence 1
  source 1..1444: mol_type = other DNA
cttggtaccatgagcctgtggaggcccagcgaggccaccgtgtacctgccccccgtgcccgtgagcaaggtggtgagcaccgacgagtacgtgaccaggaccaacatctactaccacgccggcagcgccaggctgctgaccgtgggccacccctactacagcatccccaagagcgacaaccccaagaagatcgtggtgcccaaggtgagcggcctgcagtacagggtgttcagggtgaggctgcccgaccccaacaagttcggcttccccgacaccagcttctacaaccccgagacccagaggctggtgtgggcctgcgtgggcctggaggtgggcaggggccagcccctgggcgtgggcatcagcggccaccccctgctgaacaagttcgacgacaccgagaacagcaacaggtacgccggcggccccggcaccgacaacagggagtgcatcagcatggactacaagcagacccagctgtgcctgctgggctgcaagccccccatcggcgagcactggggcaagggcagcccctgcagcaacaacgccatcacccccggcgactgcccccccctggagctgaagaacagcgtgatccaggacggcgacatggtggacaccggcttcggcgccatggacttcaccgccctgcaggacaccaagagcaacgtgcccctggacatctgcaacagcatctgcaagtaccccgactacctgaagatggtggccgagccctacggcgacaccctgttcttctacctgaggagggagcagatgttcgtgaggcacttcttcaacaggagcggcaccgtgggcgagagcgtgcccaccgacctgtacatcaagggcagcggcagcaccgccaccctggccaacagcacctacttccccacccccagcggcagcatggtgaccagcgacgcccagatcttcaacaagccctactggatgcagagggcccagggccacaacaacggcatctgctggggcaaccagctgttcgtgaccgtggtggacaccaccaggagcaccaacatgagcgtgtgcgccgccatcgccaacagcgacaccaccttcaagagcagcaacttcaaggagtacctgaggcacggcgaggagttcgacctgcagttcatcttccagctgtgcaagatcaccctgagcgccgacatcatgacctacatccacagcatgaaccccgccatcctggaggactggaacttcggcctgaccaccccccccagcggcagcctggaggacacctacaggttcgtgaccagccaggccatcacctgccagaagtccgccccccagaagcccaaggaggaccccttcaaggactacgtgttctgggaggtgaacctgaaggagaagttcagcgccgacctggaccagttccccctgggcaggaagttcctgctgcaggccggctacaaagccaagc

SEQ ID NO: 62 - DNA, 35 bp, synthetic construct
  misc_feature 1..35: note = 3110 HPV31L1 F2
  source 1..35: mol_type = other DNA
ggccggctacaaagccaagccaaaactgaaaaggg

SEQ ID NO: 63 - DNA, 41 bp, synthetic construct
  misc_feature 1..41: note = 3111 HPV31L1 R2
  source 1..41: mol_type = other DNA
ctgtctagatttacttcttcaccttcttcctcttggcagag

SEQ ID NO: 64 - DNA, 101 bp, synthetic construct
  misc_feature 1..101: note = 3112 HPV31L1 amplified sequence 2
  source 1..101: mol_type = other DNA
ggccggctacaaagccaagccaaaactgaaaagggctgccccaaccagcaccaggacctcctctgccaagaggaagaaggtgaagaagtaaatctagacag

SEQ ID NO: 65 - protein, 38 aa, Homo sapiens; identical to SEQ ID NO: 13
  REGION 1..38: note = misc_feature - 3113 Amino acid sequence of HPV type 59 L1 protein aa 471-508

SEQ ID NO: 66 - protein, 472 aa, Homo sapiens
  REGION 1..472: note = misc_feature - 3501 Amino acid sequence of HPV type 35 L1 protein aa 1-472
MSLWRSNEATVYLPPVSVSKVVSTDEYVTRTNIYYHAGSSRLLAVGHPYYAIKKQDSNKIAVPKVSGLQYRVFRVKLPDPNKFGFPDTSFYDPASQRLVWACTGVEVGRGQPLGVGISGHPLLNKLDDTENSNKYVGNSGTDNRECISMDYKQTQLCLIGCRPPIGEHWGKGTPCNANQVKAGECPPLELLNTVLQDGDMVDTGFGAMDFTTLQANKSDVPLDICSSICKYPDYLKMVSEPYGDMLFFYLRREQMFVRHLFNRAGTVGETVPADLYIKGTTGTLPSTSYFPTPSGSMVTSDAQIFNKPYWLQRAQGHNNGICWSNQLFVTVVDTTRSTNMSVCSAVSSSDSTYKNDNFKEYLRHGEEYDLQFIFQLCKITLTADVMTYIHSMNPSILEDWNFGLTPPPSGTLEDTYRYVTSQAVTCQKPSAPKPKDDPLKNYTFWEVDLKEKFSADLDQFPLGRKFLLQAGL

SEQ ID NO: 67 - protein, 26 aa, Homo sapiens
  REGION 1..26: note = misc_feature - 3502 Amino acid sequence of HPV type 33 L1 protein aa 474-499
KAKPKLKRAAPTSTRTSSAKRKKVKK

SEQ ID NO: 68 - protein, 498 aa, synthetic construct
  REGION 1..498: note = 3503 Amino acid sequence of chimeric HPV type 35 L1 protein
MSLWRSNEATVYLPPVSVSKVVSTDEYVTRTNIYYHAGSSRLLAVGHPYYAIKKQDSNKIAVPKVSGLQYRVFRVKLPDPNKFGFPDTSFYDPASQRLVWACTGVEVGRGQPLGVGISGHPLLNKLDDTENSNKYVGNSGTDNRECISMDYKQTQLCLIGCRPPIGEHWGKGTPCNANQVKAGECPPLELLNTVLQDGDMVDTGFGAMDFTTLQANKSDVPLDICSSICKYPDYLKMVSEPYGDMLFFYLRREQMFVRHLFNRAGTVGETVPADLYIKGTTGTLPSTSYFPTPSGSMVTSDAQIFNKPYWLQRAQGHNNGICWSNQLFVTVVDTTRSTNMSVCSAVSSSDSTYKNDNFKEYLRHGEEYDLQFIFQLCKITLTADVMTYIHSMNPSILEDWNFGLTPPPSGTLEDTYRYVTSQAVTCQKPSAPKPKDDPLKNYTFWEVDLKEKFSADLDQFPLGRKFLLQAGLKAKPKLKRAAPTSTRTSSAKRKKVKK

SEQ ID NO: 69 - DNA, 1498 bp, synthetic construct
  misc_feature 1..1498: note = 3504 Nucleotide sequence of chimeric HPV type 35 L1 protein
  source 1..1498: mol_type = other DNA
atgagtctgtggaggagcaatgaggctacagtctacctgcctcctgtgtctgtgagcaaggtggtgagcacagatgaatatgtgaccaggaccaacatctactaccatgctggctccagcagactgctggctgtgggacacccatactatgccatcaagaagcaggacagcaacaagattgctgtgccaaaggtgtctggactccaatacagggtgttcagggtgaaactgcctgacccaaacaagtttggctttcctgacacctccttctatgaccctgccagccagagactggtgtgggcttgtactggagtggaggtgggcaggggacaaccactgggagtgggcatctctggacacccactgctgaacaaactggatgacacagagaacagcaacaaatatgtgggcaactctggcacagacaacagggagtgtatcagtatggactacaagcagacccaactttgtctgattggctgtagacctccaattggagaacactggggcaagggcacaccatgtaatgccaaccaggtgaaggctggagagtgtcctccattggaactgctgaacacagtgctccaagatggagatatggtggacacaggctttggagctatggacttcaccaccctccaagccaacaagtctgatgtgccactggacatctgttccagcatctgtaaataccctgactacctgaaaatggtgtctgaaccatatggagatatgctgttcttctacctgaggagggaacagatgtttgtgagacacctgttcaacagggctggcacagtgggagagacagtgcctgctgacctctacatcaagggcaccacaggcaccctgccaagcacctcctactttccaacaccatctggcagtatggtgacctctgatgcccagattttcaacaagccatactggctccaaagggctcaaggacacaacaatggcatctgttggagcaaccaactttttgtgacagtggtggacaccaccaggagcaccaatatgagtgtgtgttctgctgtgtcctcctctgacagcacctacaagaatgacaacttcaaggaatacctgagacatggagaggaatatgacctccaattcatcttccaactttgtaagattaccctgacagcagatgtgatgacctacatccacagtatgaacccaagcatcttggaggactggaactttggactgacacctcctccatctggcaccttggaggacacctacagatatgtgaccagccaggctgtgacttgtcagaagccatctgccccaaagccaaaggatgacccactgaaaaactacaccttctgggaggtggacctgaaagagaagttctctgctgacctggaccagtttccactgggcaggaagttcctgctccaagcaggactgaaagccaagccaaaactgaaaagggctgccccaaccagcaccaggacctcctctgccaagaggaagaaggtgaagaagtaaa

SEQ ID NO: 70 - DNA, 1528 bp, synthetic construct
  misc_feature 1..1528: note = 3505 Synthetic HPV35 L1 gene
  source 1..1528: mol_type = other DNA
ctgggtaccatgagtctgtggaggagcaatgaggctacagtctacctgcctcctgtgtctgtgagcaaggtggtgagcacagatgaatatgtgaccaggaccaacatctactaccatgctggctccagcagactgctggctgtgggacacccatactatgccatcaagaagcaggacagcaacaagattgctgtgccaaaggtgtctggactccaatacagggtgttcagggtgaaactgcctgacccaaacaagtttggctttcctgacacctccttctatgaccctgccagccagagactggtgtgggcttgtactggagtggaggtgggcaggggacaaccactgggagtgggcatctctggacacccactgctgaacaaactggatgacacagagaacagcaacaaatatgtgggcaactctggcacagacaacagggagtgtatcagtatggactacaagcagacccaactttgtctgattggctgtagacctccaattggagaacactggggcaagggcacaccatgtaatgccaaccaggtgaaggctggagagtgtcctccattggaactgctgaacacagtgctccaagatggagatatggtggacacaggctttggagctatggacttcaccaccctccaagccaacaagtctgatgtgccactggacatctgttccagcatctgtaaataccctgactacctgaaaatggtgtctgaaccatatggagatatgctgttcttctacctgaggagggaacagatgtttgtgagacacctgttcaacagggctggcacagtgggagagacagtgcctgctgacctctacatcaagggcaccacaggcaccctgccaagcacctcctactttccaacaccatctggcagtatggtgacctctgatgcccagattttcaacaagccatactggctccaaagggctcaaggacacaacaatggcatctgttggagcaaccaactttttgtgacagtggtggacaccaccaggagcaccaatatgagtgtgtgttctgctgtgtcctcctctgacagcacctacaagaatgacaacttcaaggaatacctgagacatggagaggaatatgacctccaattcatcttccaactttgtaagattaccctgacagcagatgtgatgacctacatccacagtatgaacccaagcatcttggaggactggaactttggactgacacctcctccatctggcaccttggaggacacctacagatatgtgaccagccaggctgtgacttgtcagaagccatctgccccaaagccaaaggatgacccactgaaaaactacaccttctgggaggtggacctgaaagagaagttctctgctgacctggaccagtttccactgggcaggaagttcctgctccaagcaggactgaaagccagaccaaacttcagactgggcaagagggctgcccctgccagcaccagcaagaagtccagcaccaagaggaggaaggtgaagagctaaactcgagctc

SEQ ID NO: 71 - DNA, 1519 bp, synthetic construct; identical to SEQ ID NO: 6
  misc_feature 1..1519: note = 3506 Synthetic HPV33 L1 gene
  source 1..1519: mol_type = other DNA

SEQ ID NO: 72 - DNA, 34 bp, synthetic construct
  misc_feature 1..34: note = 3507 HPV35L1 F1
  source 1..34: mol_type = other DNA
cttggtaccatgagtctgtggaggagcaatgagg

SEQ ID NO: 73 - DNA, 36 bp, synthetic construct; identical to SEQ ID NO: 34
  misc_feature 1..36: note = 3508 HPV35L1 R1
  source 1..36: mol_type = other DNA

SEQ ID NO: 74 - DNA, 1435 bp, synthetic construct
  misc_feature 1..1435: note = 3509 HPV35L1 amplified sequence 1
  source 1..1435: mol_type = other DNA
cttggtaccatgagtctgtggaggagcaatgaggctacagtctacctgcctcctgtgtctgtgagcaaggtggtgagcacagatgaatatgtgaccaggaccaacatctactaccatgctggctccagcagactgctggctgtgggacacccatactatgccatcaagaagcaggacagcaacaagattgctgtgccaaaggtgtctggactccaatacagggtgttcagggtgaaactgcctgacccaaacaagtttggctttcctgacacctccttctatgaccctgccagccagagactggtgtgggcttgtactggagtggaggtgggcaggggacaaccactgggagtgggcatctctggacacccactgctgaacaaactggatgacacagagaacagcaacaaatatgtgggcaactctggcacagacaacagggagtgtatcagtatggactacaagcagacccaactttgtctgattggctgtagacctccaattggagaacactggggcaagggcacaccatgtaatgccaaccaggtgaaggctggagagtgtcctccattggaactgctgaacacagtgctccaagatggagatatggtggacacaggctttggagctatggacttcaccaccctccaagccaacaagtctgatgtgccactggacatctgttccagcatctgtaaataccctgactacctgaaaatggtgtctgaaccatatggagatatgctgttcttctacctgaggagggaacagatgtttgtgagacacctgttcaacagggctggcacagtgggagagacagtgcctgctgacctctacatcaagggcaccacaggcaccctgccaagcacctcctactttccaacaccatctggcagtatggtgacctctgatgcccagattttcaacaagccatactggctccaaagggctcaaggacacaacaatggcatctgttggagcaaccaactttttgtgacagtggtggacaccaccaggagcaccaatatgagtgtgtgttctgctgtgtcctcctctgacagcacctacaagaatgacaacttcaaggaatacctgagacatggagaggaatatgacctccaattcatcttccaactttgtaagattaccctgacagcagatgtgatgacctacatccacagtatgaacccaagcatcttggaggactggaactttggactgacacctcctccatctggcaccttggaggacacctacagatatgtgaccagccaggctgtgacttgtcagaagccatctgccccaaagccaaaggatgacccactgaaaaactacaccttctgggaggtggacctgaaagagaagttctctgctgacctggaccagtttccactgggcaggaagttcctgctccaagcaggactgaaagccaagc

SEQ ID NO: 75 - DNA, 35 bp, synthetic construct; identical to SEQ ID NO: 36
  misc_feature 1..35: note = 3510 HPV35L1 F2
  source 1..35: mol_type = other DNA

SEQ ID NO: 76 - DNA, 36 bp, synthetic construct; identical to SEQ ID NO: 37
  misc_feature 1..36: note = 3511 HPV35L1 R2
  source 1..36: mol_type = other DNA

SEQ ID NO: 77 - DNA, 101 bp, synthetic construct; identical to SEQ ID NO: 38
  misc_feature 1..101: note = 3512 HPV35L1 amplified sequence 2
  source 1..101: mol_type = other DNA

SEQ ID NO: 78 - protein, 38 aa, Homo sapiens; identical to SEQ ID NO: 13
  REGION 1..38: note = misc_feature - 3513 Amino acid sequence of HPV type 59 L1 protein aa 471-508

SEQ ID NO: 79 - protein, 469 aa, Homo sapiens
  REGION 1..469: note = misc_feature - 3901 Amino acid sequence of HPV type 39 L1 protein aa 1-469
MAMWRSSDSMVYLPPPSVAKVVNTDDYVTRTGIYYYAGSSRLLTVGHPYFKVGMNGGRKQDIPKVSAYQYRVFRVTLPDPNKFSIPDASLYNPETQRLVWACVGVEVGRGQPLGVGISGHPLYNRQDDTENSPFSSTTNKDSRDNVSVDYKQTQLCIIGCVPAIGEHWGKGKACKPNNVSTGDCPPLELVNTPIEDGDMIDTGYGAMDFGALQETKSEVPLDICQSICKYPDYLQMSADVYGDSMFFCLRREQLFARHFWNRGGMVGDAIPAQLYIKGTDIRANPGSSVYCPSPSGSMVTSDSQLFNKPYWLHKAQGHNNGICWHNQLFLTVVDTTRSTNFTLSTSIESSIPSTYDPSKFKEYTRHVEEYDLQFIFQLCTVTLTTDVMSYIHTMNSSILDNWNFAVAPPPSASLVDTYRYLQSAAITCQKDAPAPEKKDPYDGLKFWNVDLREKFSLELDQFPLGRKFL

SEQ ID NO: 80 - protein, 38 aa, Homo sapiens; identical to SEQ ID NO: 13
  REGION 1..38: note = misc_feature - 3902 Amino acid sequence of HPV type 59 L1 protein aa 471-508

SEQ ID NO: 81 - protein, 507 aa, synthetic construct
  REGION 1..507: note = 3903 Amino acid sequence of chimeric HPV type 39 L1 protein
MAMWRSSDSMVYLPPPSVAKVVNTDDYVTRTGIYYYAGSSRLLTVGHPYFKVGMNGGRKQDIPKVSAYQYRVFRVTLPDPNKFSIPDASLYNPETQRLVWACVGVEVGRGQPLGVGISGHPLYNRQDDTENSPFSSTTNKDSRDNVSVDYKQTQLCIIGCVPAIGEHWGKGKACKPNNVSTGDCPPLELVNTPIEDGDMIDTGYGAMDFGALQETKSEVPLDICQSICKYPDYLQMSADVYGDSMFFCLRREQLFARHFWNRGGMVGDAIPAQLYIKGTDIRANPGSSVYCPSPSGSMVTSDSQLFNKPYWLHKAQGHNNGICWHNQLFLTVVDTTRSTNFTLSTSIESSIPSTYDPSKFKEYTRHVEEYDLQFIFQLCTVTLTTDVMSYIHTMNSSILDNWNFAVAPPPSASLVDTYRYLQSAAITCQKDAPAPEKKDPYDGLKFWNVDLREKFSLELDQFPLGRKFLLQLGARPKPTIGPRKRAAPAPTSTPSPKRVKRRKSSRK

SEQ ID NO: 82 - DNA, 1525 bp, synthetic construct
  misc_feature 1..1525: note = 3904 Nucleotide sequence of chimeric HPV type 39 L1 protein
  source 1..1525: mol_type = other DNA
atggctatgtggaggtcctctgacagtatggtctacctgcctcctccatctgtggctaaggtggtgaacacagatgactatgtgaccaggacaggcatctactactatgctggctccagcagactgctgacagtgggacacccatacttcaaggtggggatgaatggaggcaggaagcaggacatcccaaaggtgtctgcctaccaatacagggtgttcagggtgaccctgcctgacccaaacaagttcagcatccctgatgcctccctctacaaccctgagacccagagactggtgtgggcttgtgtgggagtggaggtgggcaggggacaaccactgggagtgggcatctctggacacccactctacaacagacaggatgacacagagaacagcccattctccagcaccaccaacaaggacagcagggacaatgtgtctgtggactacaagcagacccaactttgtatcattggctgtgtgcctgccattggagaacactggggcaagggcaaggcttgtaagccaaacaatgtgagcacaggagactgtcctccattggaactggtgaacacaccaattgaggatggagatatgattgacacaggctatggagctatggactttggagccctccaagagaccaagtctgaggtgccactggacatctgtcagagcatctgtaaataccctgactacctccaaatgagtgctgatgtctatggagacagtatgttcttctgtctgaggagggaacaactttttgccagacacttctggaacaggggagggatggtgggagatgccatccctgcccaactctacatcaagggcacagacatcagggctaaccctggctcctctgtctactgtccaagcccatctggcagtatggtgacctctgacagccaacttttcaacaagccatactggctgcacaaggctcaaggacacaacaatggcatctgttggcacaaccaacttttcctgacagtggtggacaccaccaggagcaccaacttcaccctgagcaccagcattgagtccagcatcccaagcacctatgacccaagcaagttcaaggaatacaccaggcatgtggaggaatatgacctccaattcatcttccaactttgtactgtgaccctgaccacagatgtgatgagttacatccacacaatgaactccagcatcctggacaactggaactttgctgtggctcctcctccatctgcctccctggtggacacctacagatacctccaatctgctgccatcacttgtcagaaggatgcccctgcccctgagaagaaggacccatatgatggactgaagttctggaatgtggacctgagggagaagttctccttggaactggaccagtttccactgggcaggaagttcctgctccaacttggagccagaccaaagccaaccattggaccaaggaagagggctgcccctgccccaaccagcacaccaagcccaaagagggtgaagaggaggaagtccagcaggaagtaaa

SEQ ID NO: 83 - DNA, 1537 bp, synthetic construct
  misc_feature 1..1537: note = 3905 Synthetic HPV39 L1 gene
  source 1..1537: mol_type = other DNA
ctgggtaccatggctatgtggaggtcctctgacagtatggtctacctgcctcctccatctgtggctaaggtggtgaacacagatgactatgtgaccaggacaggcatctactactatgctggctccagcagactgctgacagtgggacacccatacttcaaggtggggatgaatggaggcaggaagcaggacatcccaaaggtgtctgcctaccaatacagggtgttcagggtgaccctgcctgacccaaacaagttcagcatccctgatgcctccctctacaaccctgagacccagagactggtgtgggcttgtgtgggagtggaggtgggcaggggacaaccactgggagtgggcatctctggacacccactctacaacagacaggatgacacagagaacagcccattctccagcaccaccaacaaggacagcagggacaatgtgtctgtggactacaagcagacccaactttgtatcattggctgtgtgcctgccattggagaacactggggcaagggcaaggcttgtaagccaaacaatgtgagcacaggagactgtcctccattggaactggtgaacacaccaattgaggatggagatatgattgacacaggctatggagctatggactttggagccctccaagagaccaagtctgaggtgccactggacatctgtcagagcatctgtaaataccctgactacctccaaatgagtgctgatgtctatggagacagtatgttcttctgtctgaggagggaacaactttttgccagacacttctggaacaggggagggatggtgggagatgccatccctgcccaactctacatcaagggcacagacatcagggctaaccctggctcctctgtctactgtccaagcccatctggcagtatggtgacctctgacagccaacttttcaacaagccatactggctgcacaaggctcaaggacacaacaatggcatctgttggcacaaccaacttttcctgacagtggtggacaccaccaggagcaccaacttcaccctgagcaccagcattgagtccagcatcccaagcacctatgacccaagcaagttcaaggaatacaccaggcatgtggaggaatatgacctccaattcatcttccaactttgtactgtgaccctgaccacagatgtgatgagttacatccacacaatgaactccagcatcctggacaactggaactttgctgtggctcctcctccatctgcctccctggtggacacctacagatacctccaatctgctgccatcacttgtcagaaggatgcccctgcccctgagaagaaggacccatatgatggactgaagttctggaatgtggacctgagggagaagttctccttggaactggaccagtttccactgggcaggaagttcctgctccaagccagggtgaggaggagaccaaccattggaccaaggaagagacctgctgccagcacctcctcctcctctgccaccaaacacaagaggaagagggtgagcaagtaaactcgagctc

SEQ ID NO: 84 - DNA, 1546 bp, synthetic construct
  misc_feature 1..1546: note = 3906 Synthetic HPV59 L1 gene
  source 1..1546: mol_type = other DNA
ctgggtaccatggctctgtggaggtcctctgacaacaaggtctacctgcctcctccatctgtggctaaggtggtgagcacagatgaatatgtgaccaggaccagcatcttctaccatgctggctccagcagactgctgacagtgggacacccatacttcaaggtgccaaagggaggcaatggcagacaggatgtgccaaaggtgtctgcctaccaatacagggtgttcagggtgaaactgcctgacccaaacaagtttggactgcctgacaacacagtctatgacccaaacagccagagactggtgtgggcttgtgtgggagtggagattggcaggggacaaccactgggagtgggactgtctggacacccactctacaacaaactggatgacacagagaactctcatgtggcatctgctgtggacaccaaggacaccagggacaatgtgtctgtggactacaagcagacccaactttgtatcattggctgtgtgcctgccattggagaacactggaccaagggcacagcctgtaagccaaccacagtggtccagggagactgtcctccattggaactgataaacacaccaattgaggatggagatatggtggacacaggctatggagctatggacttcaaactgctccaagacaacaagtctgaggtgccactggacatctgtcagagcatctgtaaataccctgactacctccaaatgagtgctgatgcctatggagacagtatgttcttctgtctgaggagggaacaggtgtttgccagacacttctggaacaggtctggcacaatgggagaccaacttcctgagtccctctacatcaagggcacagacatcagggctaaccctggctcctacctctacagcccaagcccatctggctctgtggtgacctctgacagccaacttttcaacaagccatactggctgcacaaggctcaaggactgaacaatggcatctgttggcacaaccaacttttcctgacagtggtggacaccaccaggagcaccaacctgtctgtgtgtgccagcaccacctccagcatcccaaatgtctacacaccaacctccttcaaggaatatgccaggcatgtggaggagtttgacctccaattcatcttccaactttgtaagattaccctgaccacagaggtgatgagttacatccacaatatgaacaccaccatcttggaggactggaactttggagtgacacctcctccaacagcctccctggtggacacctacaggtttgtccagtctgctgctgtgacttgtcagaaggacacagcccctcctgtgaagcaggacccatatgacaaactgaagttctggcctgtggacctgaaagagaggttctctgctgacctggaccagtttccactgggcaggaagttcctgctccaacttggagccagaccaaagccaaccattggaccaaggaagagggctgcccctgccccaaccagcacaccaagcccaaagagggtgaagaggaggaagtccagcaggaagtaaactcgagctc

SEQ ID NO: 85 - DNA, 38 bp, synthetic construct
  misc_feature 1..38: note = 3907 HPV39L1 F1
  source 1..38: mol_type = other DNA
cttggtaccatggctatgtggaggtcctctgacagtat

SEQ ID NO: 86 - DNA, 38 bp, synthetic construct
  misc_feature 1..38: note = 3908 HPV39L1 R1
  source 1..38: mol_type = other DNA
tccaagttggagcaggaacttcctgcccagtggaaact

SEQ ID NO: 87 - DNA, 1428 bp, synthetic construct
  misc_feature 1..1428: note = 3909 HPV39L1 amplified sequence 1
  source 1..1428: mol_type = other DNA
cttggtaccatggctatgtggaggtcctctgacagtatggtctacctgcctcctccatctgtggctaaggtggtgaacacagatgactatgtgaccaggacaggcatctactactatgctggctccagcagactgctgacagtgggacacccatacttcaaggtggggatgaatggaggcaggaagcaggacatcccaaaggtgtctgcctaccaatacagggtgttcagggtgaccctgcctgacccaaacaagttcagcatccctgatgcctccctctacaaccctgagacccagagactggtgtgggcttgtgtgggagtggaggtgggcaggggacaaccactgggagtgggcatctctggacacccactctacaacagacaggatgacacagagaacagcccattctccagcaccaccaacaaggacagcagggacaatgtgtctgtggactacaagcagacccaactttgtatcattggctgtgtgcctgccattggagaacactggggcaagggcaaggcttgtaagccaaacaatgtgagcacaggagactgtcctccattggaactggtgaacacaccaattgaggatggagatatgattgacacaggctatggagctatggactttggagccctccaagagaccaagtctgaggtgccactggacatctgtcagagcatctgtaaataccctgactacctccaaatgagtgctgatgtctatggagacagtatgttcttctgtctgaggagggaacaactttttgccagacacttctggaacaggggagggatggtgggagatgccatccctgcccaactctacatcaagggcacagacatcagggctaaccctggctcctctgtctactgtccaagcccatctggcagtatggtgacctctgacagccaacttttcaacaagccatactggctgcacaaggctcaaggacacaacaatggcatctgttggcacaaccaacttttcctgacagtggtggacaccaccaggagcaccaacttcaccctgagcaccagcattgagtccagcatcccaagcacctatgacccaagcaagttcaaggaatacaccaggcatgtggaggaatatgacctccaattcatcttccaactttgtactgtgaccctgaccacagatgtgatgagttacatccacacaatgaactccagcatcctggacaactggaactttgctgtggctcctcctccatctgcctccctggtggacacctacagatacctccaatctgctgccatcacttgtcagaaggatgcccctgcccctgagaagaaggacccatatgatggactgaagttctggaatgtggacctgagggagaagttctccttggaactggaccagtttccactgggcaggaagttcctgctccaacttgga

SEQ ID NO: 88 - DNA, 37 bp, synthetic construct
  misc_feature 1..37: note = 3910 HPV39L1 F2
  source 1..37: mol_type = other DNA
aggaagttcctgctccaacttggagccagaccaaagc

SEQ ID NO: 89 - DNA, 40 bp, synthetic construct
  misc_feature 1..40: note = 3911 HPV39L1 R2
  source 1..40: mol_type = other DNA
ctgtctagatttacttcctgctggacttcctcctcttcac

SEQ ID NO: 90 - DNA, 139 bp, synthetic construct
  misc_feature 1..139: note = 3912 HPV39L1 amplified sequence 2
  source 1..139: mol_type = other DNA
aggaagttcctgctccaacttggagccagaccaaagccaaccattggaccaaggaagagggctgcccctgccccaaccagcacaccaagcccaaagagggtgaagaggaggaagtccagcaggaagtaaatctagacag

SEQ ID NO: 91 - protein, 26 aa, Homo sapiens
  REGION 1..26: note = misc_feature - 3913 Amino acid sequence of HPV type 33 L1 protein aa 474-499
KAKPKLKRAAPTSTRTSSAKRKKVKK

SEQ ID NO: 92 - protein, 478 aa, Homo sapiens
  REGION 1..478: note = misc_feature - 4501 Amino acid sequence of HPV type 45 L1 protein aa 1-478
MALWRPSDSTVYLPPPSVARVVNTDDYVSRTSIFYHAGSSRLLTVGNPYFRVVPSGAGNKQAVPKVSAYQYRVFRVALPDPNKFGLPDSTIYNPETQRLVWACVGMEIGRGQPLGIGLSGHPFYNKLDDTESAHAATAVITQDVRDNVSVDYKQTQLCILGCVPAIGEHWAKGTLCKPAQLQPGDCPPLELKNTIIEDGDMVDTGYGAMDFSTLQDTKCEVPLDICQSICKYPDYLQMSADPYGDSMFFCLRREQLFARHFWNRAGVMGDTVPTDLYIKGTSANMRETPGSCVYSPSPSGSITTSDSQLFNKPYWLHKAQGHNNGICWHNQLFVTVVDTTRSTNLTLCASTQNPVPNTYDPTKFKHYSRHVEEYDLQFIFQLCTITLTAEVMSYIHSMNSSILENWNFGVPPPPTTSLVDTYRFVQSVAVTCQKDTTPPEKQDPYDKLKFWTVDLKEKFSSDLDQYPLGRKFLVQAGL

SEQ ID NO: 93 - protein, 26 aa, Homo sapiens
  REGION 1..26: note = misc_feature - 4502 Amino acid sequence of HPV type 33 L1 protein aa 474-499
KAKPKLKRAAPTSTRTSSAKRKKVKK

SEQ ID NO: 94 - protein, 504 aa, synthetic construct
  REGION 1..504: note = 4503 Amino acid sequence of chimeric HPV type 45 L1 protein
MALWRPSDSTVYLPPPSVARVVNTDDYVSRTSIFYHAGSSRLLTVGNPYFRVVPSGAGNKQAVPKVSAYQYRVFRVALPDPNKFGLPDSTIYNPETQRLVWACVGMEIGRGQPLGIGLSGHPFYNKLDDTESAHAATAVITQDVRDNVSVDYKQTQLCILGCVPAIGEHWAKGTLCKPAQLQPGDCPPLELKNTIIEDGDMVDTGYGAMDFSTLQDTKCEVPLDICQSICKYPDYLQMSADPYGDSMFFCLRREQLFARHFWNRAGVMGDTVPTDLYIKGTSANMRETPGSCVYSPSPSGSITTSDSQLFNKPYWLHKAQGHNNGICWHNQLFVTVVDTTRSTNLTLCASTQNPVPNTYDPTKFKHYSRHVEEYDLQFIFQLCTITLTAEVMSYIHSMNSSILENWNFGVPPPPTTSLVDTYRFVQSVAVTCQKDTTPPEKQDPYDKLKFWTVDLKEKFSSDLDQYPLGRKFLVQAGLKAKPKLKRAAPTSTRTSSAKRKKVKK

SEQ ID NO: 95 - DNA, 1516 bp, synthetic construct
  misc_feature 1..1516: note = 4504 Nucleotide sequence of chimeric HPV type 45 L1 protein
  source 1..1516: mol_type = other DNA
atggctctgtggagaccatctgacagcacagtctacctgcctcctccatctgtggcaagggtggtgaacacagatgactatgtgagcaggaccagcatcttctaccatgctggctccagcagactgctgacagtgggcaacccatacttcagggtggtgccaagtggagcaggcaacaagcaggctgtgccaaaggtgtctgcctaccaatacagggtgttcagggtggctctgcctgacccaaacaagtttggactgcctgacagcaccatctacaaccctgagacccagagactggtgtgggcttgtgtggggatggagattggcaggggacaaccactgggcattggactgtctggacacccattctacaacaaactggatgacacagagtctgcccatgctgccacagcagtgattacccaggatgtgagggacaatgtgtctgtggactacaagcagacccaactttgtatcctgggctgtgtgcctgccattggagaacactgggctaagggcaccctgtgtaagcctgcccaactccaacctggagactgtcctccattggaactgaaaaacaccatcattgaggatggagatatggtggacacaggctatggagctatggacttcagcaccctccaagacaccaagtgtgaggtgccactggacatctgtcagagcatctgtaaataccctgactacctccaaatgagtgctgacccatatggagacagtatgttcttctgtctgaggagggaacaactttttgccagacacttctggaacagggctggagtgatgggagacacagtgccaacagacctctacatcaagggcacctctgccaatatgagggagacacctggctcctgtgtctacagcccaagcccatctggcagcatcaccacctctgacagccaacttttcaacaagccatactggctgcacaaggctcaaggacacaacaatggcatctgttggcacaaccaactttttgtgacagtggtggacaccaccaggagcaccaacctgaccctgtgtgccagcacccagaaccctgtgccaaacacctatgacccaaccaagttcaagcactacagcaggcatgtggaggaatatgacctccaattcatcttccaactttgtaccatcaccctgacagcagaggtgatgagttacatccacagtatgaactccagcatcttggagaactggaactttggagtgcctcctcctccaaccacctccctggtggacacctacaggtttgtccagtctgtggctgtgacttgtcagaaggacaccacacctcctgagaagcaggacccatatgacaaactgaagttctggacagtggacctgaaagagaagttctcctctgacctggaccaatacccactgggcaggaagttcctggtccaggctggactgaaagccaagccaaaactgaaaagggctgccccaaccagcaccaggacctcctctgccaagaggaagaaggtgaagaagtaaa

SEQ ID NO: 96 - DNA, 1552 bp, synthetic construct
  misc_feature 1..1552: note = 4505 Synthetic HPV45 L1 gene
  source 1..1552: mol_type = other DNA
ctgggtaccatggctctgtggagaccatctgacagcacagtctacctgcctcctccatctgtggcaagggtggtgaacacagatgactatgtgagcaggaccagcatcttctaccatgctggctccagcagactgctgacagtgggcaacccatacttcagggtggtgccaagtggagcaggcaacaagcaggctgtgccaaaggtgtctgcctaccaatacagggtgttcagggtggctctgcctgacccaaacaagtttggactgcctgacagcaccatctacaaccctgagacccagagactggtgtgggcttgtgtggggatggagattggcaggggacaaccactgggcattggactgtctggacacccattctacaacaaactggatgacacagagtctgcccatgctgccacagcagtgattacccaggatgtgagggacaatgtgtctgtggactacaagcagacccaactttgtatcctgggctgtgtgcctgccattggagaacactgggctaagggcaccctgtgtaagcctgcccaactccaacctggagactgtcctccattggaactgaaaaacaccatcattgaggatggagatatggtggacacaggctatggagctatggacttcagcaccctccaagacaccaagtgtgaggtgccactggacatctgtcagagcatctgtaaataccctgactacctccaaatgagtgctgacccatatggagacagtatgttcttctgtctgaggagggaacaactttttgccagacacttctggaacagggctggagtgatgggagacacagtgccaacagacctctacatcaagggcacctctgccaatatgagggagacacctggctcctgtgtctacagcccaagcccatctggcagcatcaccacctctgacagccaacttttcaacaagccatactggctgcacaaggctcaaggacacaacaatggcatctgttggcacaaccaactttttgtgacagtggtggacaccaccaggagcaccaacctgaccctgtgtgccagcacccagaaccctgtgccaaacacctatgacccaaccaagttcaagcactacagcaggcatgtggaggaatatgacctccaattcatcttccaactttgtaccatcaccctgacagcagaggtgatgagttacatccacagtatgaactccagcatcttggagaactggaactttggagtgcctcctcctccaaccacctccctggtggacacctacaggtttgtccagtctgtggctgtgacttgtcagaaggacaccacacctcctgagaagcaggacccatatgacaaactgaagttctggacagtggacctgaaagagaagttctcctctgacctggaccaatacccactgggcaggaagttcctggtccaggctggactgaggaggagaccaaccattggaccaaggaagagacctgctgccagcaccagcacagccagcagacctgccaagagggtgaggattaggagcaagaagtaaactcgagctc

SEQ ID NO: 97 - DNA, 1519 bp, synthetic construct; identical to SEQ ID NO: 6
  misc_feature 1..1519: note = 4506 Synthetic HPV33 L1 gene
  source 1..1519: mol_type = other DNA

SEQ ID NO: 98 - DNA, 33 bp, synthetic construct
  misc_feature 1..33: note = 4507 HPV45L1 F1
  source 1..33: mol_type = other DNA
cttggtaccatggctctgtggagaccatctgac

SEQ ID NO: 99 - DNA, 32 bp, synthetic construct
  misc_feature 1..32: note = 4508 HPV45L1 R1
  source 1..32: mol_type = other DNA
gcttggctttcagtccagcctggaccaggaac

SEQ ID NO: 100 - DNA, 1453 bp, synthetic construct
  misc_feature 1..1453: note = 4509 HPV45L1 amplified sequence 1
  source 1..1453: mol_type = other DNA
cttggtaccatggctctgtggagaccatctgacagcacagtctacctgcctcctccatctgtggcaagggtggtgaacacagatgactatgtgagcaggaccagcatcttctaccatgctggctccagcagactgctgacagtgggcaacccatacttcagggtggtgccaagtggagcaggcaacaagcaggctgtgccaaaggtgtctgcctaccaatacagggtgttcagggtggctctgcctgacccaaacaagtttggactgcctgacagcaccatctacaaccctgagacccagagactggtgtgggcttgtgtggggatggagattggcaggggacaaccactgggcattggactgtctggacacccattctacaacaaactggatgacacagagtctgcccatgctgccacagcagtgattacccaggatgtgagggacaatgtgtctgtggactacaagcagacccaactttgtatcctgggctgtgtgcctgccattggagaacactgggctaagggcaccctgtgtaagcctgcccaactccaacctggagactgtcctccattggaactgaaaaacaccatcattgaggatggagatatggtggacacaggctatggagctatggacttcagcaccctccaagacaccaagtgtgaggtgccactggacatctgtcagagcatctgtaaataccctgactacctccaaatgagtgctgacccatatggagacagtatgttcttctgtctgaggagggaacaactttttgccagacacttctggaacagggctggagtgatgggagacacagtgccaacagacctctacatcaagggcacctctgccaatatgagggagacacctggctcctgtgtctacagcccaagcccatctggcagcatcaccacctctgacagccaacttttcaacaagccatactggctgcacaaggctcaaggacacaacaatggcatctgttggcacaaccaactttttgtgacagtggtggacaccaccaggagcaccaacctgaccctgtgtgccagcacccagaaccctgtgccaaacacctatgacccaaccaagttcaagcactacagcaggcatgtggaggaatatgacctccaattcatcttccaactttgtaccatcaccctgacagcagaggtgatgagttacatccacagtatgaactccagcatcttggagaactggaactttggagtgcctcctcctccaaccacctccctggtggacacctacaggtttgtccagtctgtggctgtgacttgtcagaaggacaccacacctcctgagaagcaggacccatatgacaaactgaagttctggacagtggacctgaaagagaagttctcctctgacctggaccaatacccactgggcaggaagttcctggtccaggctggactgaaagccaagc

SEQ ID NO: 101 - DNA, 35 bp, synthetic construct
  misc_feature 1..35: note = 4510 HPV45L1 F2
  source 1..35: mol_type = other DNA
ggctggactgaaagccaagccaaaactgaaaaggg

SEQ ID NO: 102 - DNA, 37 bp, synthetic construct; identical to SEQ ID NO: 11
  misc_feature 1..37: note = 4511 HPV45L1 R2
  source 1..37: mol_type = other DNA

SEQ ID NO: 103 - DNA, 101 bp, synthetic construct
  misc_feature 1..101: note = 4512 HPV45L1 amplified sequence 2
  source 1..101: mol_type = other DNA
ggctggactgaaagccaagccaaaactgaaaagggctgccccaaccagcaccaggacctcctctgccaagaggaagaaggtgaagaagtaaatctagacag

SEQ ID NO: 104 - protein, 38 aa, Homo sapiens; identical to SEQ ID NO: 13
  REGION 1..38: note = misc_feature - 4513 Amino acid sequence of HPV type 59 L1 protein aa 471-508

SEQ ID NO: 105 - protein, 474 aa, Homo sapiens
  REGION 1..474: note = misc_feature - 5101 Amino acid sequence of HPV type 51 L1 protein aa 1-474
MALWRTNDSKVYLPPAPVSRIVNTEEYITRTGIYYYAGSSRLITLGHPYFPIPKTSTRAAIPKVSAFQYRVFRVQLPDPNKFGLPDPNLYNPDTDRLVWGCVGVEVGRGQPLGVGLSGHPLFNKYDDTENSRIANGNAQQDVRDNTSVDNKQTQLCIIGCAPPIGEHWGIGTTCKNTPVPPGDCPPLELVSSVIQDGDMIDTGFGAMDFAALQATKSDVPLDISQSVCKYPDYLKMSADTYGNSMFFHLRREQIFARHYYNKLVGVGEDIPNDYYIKGSGNGRDPIESYIYSATPSGSMITSDSQIFNKPYWLHRAQGHNNGICWNNQLFITCVDTTRSTNLTISTATAAVSPTFTPSNFKQYIRHGEEYELQFIFQLCKITLTTEVMAYLHTMDPTILEQWNFGLTLPPSASLEDAYRFVRNAATSCQKDTPPQAKPDPLAKYKFWDVDLKERFSLDLDQFALGRKFLLQVGV

SEQ ID NO: 106 - protein, 26 aa, Homo sapiens
  REGION 1..26: note = misc_feature - 5102 Amino acid sequence of HPV type 33 L1 protein aa 474-499
KAKPKLKRAAPTSTRTSSAKRKKVKK

SEQ ID NO: 107 - protein, 500 aa, synthetic construct
  REGION 1..500: note = 5103 Amino acid sequence of chimeric HPV type 51 L1 protein
MALWRTNDSKVYLPPAPVSRIVNTEEYITRTGIYYYAGSSRLITLGHPYFPIPKTSTRAAIPKVSAFQYRVFRVQLPDPNKFGLPDPNLYNPDTDRLVWGCVGVEVGRGQPLGVGLSGHPLFNKYDDTENSRIANGNAQQDVRDNTSVDNKQTQLCIIGCAPPIGEHWGIGTTCKNTPVPPGDCPPLELVSSVIQDGDMIDTGFGAMDFAALQATKSDVPLDISQSVCKYPDYLKMSADTYGNSMFFHLRREQIFARHYYNKLVGVGEDIPNDYYIKGSGNGRDPIESYIYSATPSGSMITSDSQIFNKPYWLHRAQGHNNGICWNNQLFITCVDTTRSTNLTISTATAAVSPTFTPSNFKQYIRHGEEYELQFIFQLCKITLTTEVMAYLHTMDPTILEQWNFGLTLPPSASLEDAYRFVRNAATSCQKDTPPQAKPDPLAKYKFWDVDLKERFSLDLDQFALGRKFLLQVGVKAKPKLKRAAPTSTRTSSAKRKKVKK

SEQ ID NO: 108 - DNA, 1504 bp, synthetic construct
  misc_feature 1..1504: note = 5104 Nucleotide sequence of chimeric HPV type 51 L1 protein
  source 1..1504: mol_type = other DNA
atggctctgtggaggaccaatgacagcaaggtctacctgcctcctgcccctgtgagcaggattgtgaacacagaggaatacatcaccaggacaggcatctactactatgctggctccagcagactgattaccctgggacacccatactttccaatcccaaagaccagcaccagggctgccatcccaaaggtgtctgccttccaatacagggtgttcagggtccaacttcctgacccaaacaagtttggactgcctgacccaaacctctacaaccctgacacagacagactggtgtggggctgtgtgggagtggaggtgggcaggggacaaccactgggagtgggactgtctggacacccactgttcaacaaatatgatgacacagagaacagcaggattgccaatggcaatgcccaacaggatgtgagggacaacacctctgtggacaacaagcagacccaactttgtatcattggctgtgcccctccaattggagaacactggggcattggcaccacttgtaagaacacacctgtgcctcctggagactgtcctccattggaactggtgtcctctgtgattcaggatggagatatgattgacacaggctttggagctatggactttgctgccctccaagccaccaagtctgatgtgccactggacatcagccagtctgtgtgtaaataccctgactacctgaaaatgagtgctgacacctatggcaacagtatgttcttccacctgaggagggaacagatttttgccagacactactacaacaaactggtgggagtgggagaggacatcccaaatgactactacatcaagggctctggcaatggcagggacccaattgagtcctacatctactctgccacaccatctggcagtatgattacctctgacagccagattttcaacaagccatactggctgcacagggctcaaggacacaacaatggcatctgttggaacaaccaacttttcatcacttgtgtggacaccaccaggagcaccaacctgaccatcagcacagccacagcagcagtgagcccaaccttcacaccaagcaacttcaagcaatacatcagacatggagaggaatatgaactccaattcatcttccaactttgtaagattaccctgaccacagaggtgatggcttacctgcacacaatggacccaaccatcttggaacagtggaactttggactgaccctgcctccatctgcctccttggaggatgcctacaggtttgtgaggaatgctgccacctcctgtcagaaggacacacctccacaggctaagcctgacccactggctaaatacaagttctgggatgtggacctgaaagagaggttctccctggacctggaccagtttgccctgggcaggaagttcctgctccaagtgggagtcaaagccaagccaaaactgaaaagggctgccccaaccagcaccaggacctcctctgccaagaggaagaaggtgaagaagtaaa

SEQ ID NO: 109 - DNA, 1534 bp, synthetic construct
  misc_feature 1..1534: note = 5105 Synthetic HPV51 L1 gene
  source 1..1534: mol_type = other DNA
ctgggtaccatggctctgtggaggaccaatgacagcaaggtctacctgcctcctgcccctgtgagcaggattgtgaacacagaggaatacatcaccaggacaggcatctactactatgctggctccagcagactgattaccctgggacacccatactttccaatcccaaagaccagcaccagggctgccatcccaaaggtgtctgccttccaatacagggtgttcagggtccaacttcctgacccaaacaagtttggactgcctgacccaaacctctacaaccctgacacagacagactggtgtggggctgtgtgggagtggaggtgggcaggggacaaccactgggagtgggactgtctggacacccactgttcaacaaatatgatgacacagagaacagcaggattgccaatggcaatgcccaacaggatgtgagggacaacacctctgtggacaacaagcagacccaactttgtatcattggctgtgcccctccaattggagaacactggggcattggcaccacttgtaagaacacacctgtgcctcctggagactgtcctccattggaactggtgtcctctgtgattcaggatggagatatgattgacacaggctttggagctatggactttgctgccctccaagccaccaagtctgatgtgccactggacatcagccagtctgtgtgtaaataccctgactacctgaaaatgagtgctgacacctatggcaacagtatgttcttccacctgaggagggaacagatttttgccagacactactacaacaaactggtgggagtgggagaggacatcccaaatgactactacatcaagggctctggcaatggcagggacccaattgagtcctacatctactctgccacaccatctggcagtatgattacctctgacagccagattttcaacaagccatactggctgcacagggctcaaggacacaacaatggcatctgttggaacaaccaacttttcatcacttgtgtggacaccaccaggagcaccaacctgaccatcagcacagccacagcagcagtgagcccaaccttcacaccaagcaacttcaagcaatacatcagacatggagaggaatatgaactccaattcatcttccaactttgtaagattaccctgaccacagaggtgatggcttacctgcacacaatggacccaaccatcttggaacagtggaactttggactgaccctgcctccatctgcctccttggaggatgcctacaggtttgtgaggaatgctgccacctcctgtcagaaggacacacctccacaggctaagcctgacccactggctaaatacaagttctgggatgtggacctgaaagagaggttctccctggacctggaccagtttgccctgggcaggaagttcctgctccaagtgggagtccagaggaagccaagacctggactgaaaagacctgcctcctctgcctcctcctcctcctcctcctctgccaagaggaagagggtgaagaagtaaactcgagctc

SEQ ID NO: 110 - DNA, 1519 bp, synthetic construct; identical to SEQ ID NO: 6
  misc_feature 1..1519: note = 5106 Synthetic HPV33 L1 gene
  source 1..1519: mol_type = other DNA

SEQ ID NO: 111 - DNA, 34 bp, synthetic construct
  misc_feature 1..34: note = 5107 HPV51L1 F1
  source 1..34: mol_type = other DNA
cttggtaccatggctctgtggaggaccaatgaca

SEQ ID NO: 112 - DNA, 32 bp, synthetic construct
  misc_feature 1..32: note = 5108 HPV51L1 R1
  source 1..32: mol_type = other DNA
gcttggctttgactcccacttggagcaggaac

SEQ ID NO: 113 - DNA, 1441 bp, synthetic construct
  misc_feature 1..1441: note = 5109 HPV51L1 amplified sequence 1
  source 1..1441: mol_type = other DNA
cttggtaccatggctctgtggaggaccaatgacagcaaggtctacctgcctcctgcccctgtgagcaggattgtgaacacagaggaatacatcaccaggacaggcatctactactatgctggctccagcagactgattaccctgggacacccatactttccaatcccaaagaccagcaccagggctgccatcccaaaggtgtctgccttccaatacagggtgttcagggtccaacttcctgacccaaacaagtttggactgcctgacccaaacctctacaaccctgacacagacagactggtgtggggctgtgtgggagtggaggtgggcaggggacaaccactgggagtgggactgtctggacacccactgttcaacaaatatgatgacacagagaacagcaggattgccaatggcaatgcccaacaggatgtgagggacaacacctctgtggacaacaagcagacccaactttgtatcattggctgtgcccctccaattggagaacactggggcattggcaccacttgtaagaacacacctgtgcctcctggagactgtcctccattggaactggtgtcctctgtgattcaggatggagatatgattgacacaggctttggagctatggactttgctgccctccaagccaccaagtctgatgtgccactggacatcagccagtctgtgtgtaaataccctgactacctgaaaatgagtgctgacacctatggcaacagtatgttcttccacctgaggagggaacagatttttgccagacactactacaacaaactggtgggagtgggagaggacatcccaaatgactactacatcaagggctctggcaatggcagggacccaattgagtcctacatctactctgccacaccatctggcagtatgattacctctgacagccagattttcaacaagccatactggctgcacagggctcaaggacacaacaatggcatctgttggaacaaccaacttttcatcacttgtgtggacaccaccaggagcaccaacctgaccatcagcacagccacagcagcagtgagcccaaccttcacaccaagcaacttcaagcaatacatcagacatggagaggaatatgaactccaattcatcttccaactttgtaagattaccctgaccacagaggtgatggcttacctgcacacaatggacccaaccatcttggaacagtggaactttggactgaccctgcctccatctgcctccttggaggatgcctacaggtttgtgaggaatgctgccacctcctgtcagaaggacacacctccacaggctaagcctgacccactggctaaatacaagttctgggatgtggacctgaaagagaggttctccctggacctggaccagtttgccctgggcaggaagttcctgctccaagtgggagtcaaagccaagc

SEQ ID NO: 114 - DNA, 35 bp, synthetic construct
  misc_feature 1..35: note = 5110 HPV51L1 F2
  source 1..35: mol_type = other DNA
agtgggagtcaaagccaagccaaaactgaaaaggg

SEQ ID NO: 115 - DNA, 36 bp, synthetic construct; identical to SEQ ID NO: 37
  misc_feature 1..36: note = 5111 HPV51L1 R2
  source 1..36: mol_type = other DNA

SEQ ID NO: 116 - DNA, 101 bp, synthetic construct
  misc_feature 1..101: note = 5112 HPV51L1 amplified sequence 2
  source 1..101: mol_type = other DNA
agtgggagtcaaagccaagccaaaactgaaaagggctgccccaaccagcaccaggacctcctctgccaagaggaagaaggtgaagaagtaaatctagacag

SEQ ID NO: 117 - protein, 38 aa, Homo sapiens; identical to SEQ ID NO: 13
  REGION 1..38: note = misc_feature - 5113 Amino acid sequence of HPV type 59 L1 protein aa 471-508

SEQ ID NO: 118 - protein, 478 aa, Homo sapiens
  REGION 1..478: note = misc_feature - 5201 Amino acid sequence of HPV type 52 L1 protein aa 1-478
MSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKDYMFWEVDLKEKFSADLDQFPLGRKFLLQAGL

SEQ ID NO: 119 - protein, 26 aa, Homo sapiens
  REGION 1..26: note = misc_feature - 5202 Amino acid sequence of HPV type 33 L1 protein aa 474-499
KAKPKLKRAAPTSTRTSSAKRKKVKK

SEQ ID NO: 120 - protein, 504 aa, synthetic construct
  REGION 1..504: note = 5203 Amino acid sequence of chimeric HPV type 52 L1 protein
MSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKDYMFWEVDLKEKFSADLDQFPLGRKFLLQAGLKAKPKLKRAAPTSTRTSSAKRKKVKK

SEQ ID NO: 121 - DNA, 1516 bp, synthetic construct
  misc_feature 1..1516: note = 5204 Nucleotide sequence of chimeric HPV type 52 L1 protein
  source 1..1516: mol_type = other DNA
atgagcgtgtggaggcccagcgaggccaccgtgtacctgccccccgtgcccgtgagcaaggtggtgagcaccgacgagtacgtgagcaggaccagcatctactactacgccggcagcagcaggctgctgaccgtgggccacccctacttcagcatcaagaacaccagcagcggcaacggcaagaaggtgctggtgcccaaggtgagcggcctgcagtacagggtgttcaggatcaagctgcccgaccccaacaagttcggcttccccgacaccagcttctacaaccccgagacccagaggctggtgtgggcctgcaccggcctggagatcggcaggggccagcccctgggcgtgggcatcagcggccaccccctgctgaacaagttcgacgacaccgagaccagcaacaagtacgccggcaagcccggcatcgacaacagggagtgcctgagcatggactacaagcagacccagctgtgcatcctgggctgcaagccccccatcggcgagcactggggcaagggcaccccctgcaacaacaacagcggcaaccccggcgactgcccccccctgcagctgatcaacagcgtgatccaggacggcgacatggtggacaccggcttcggctgcatggacttcaacaccctgcaggccagcaagagcgacgtgcccatcgacatctgcagcagcgtgtgcaagtaccccgactacctgcagatggccagcgagccctacggcgacagcctgttcttcttcctgaggagggagcagatgttcgtgaggcacttcttcaacagggccggcaccctgggcgaccccgtgcccggcgacctgtacatccagggcagcaacagcggcaacaccgccaccgtgcagagcagcgccttcttccccacccccagcggcagcatggtgaccagcgagagccagctgttcaacaagccctactggctgcagagggcccagggccacaacaacggcatctgctggggcaaccagctgttcgtgaccgtggtggacaccaccaggagcaccaacatgaccctgtgcgccgaggtgaagaaggagagcacctacaagaacgagaacttcaaggagtacctgaggcacggcgaggagttcgacctgcagttcatcttccagctgtgcaagatcaccctgaccgccgacgtgatgacctacatccacaagatggacgccaccatcctggaggactggcagttcggcctgacccccccccccagcgccagcctggaggacacctacaggttcgtgaccagcaccgccatcacctgccagaagaacaccccccccaagggcaaggaggaccccctgaaggactacatgttctgggaggtggacctgaaggagaagttcagcgccgacctggaccagttccccctgggcaggaagttcctgctgcaggccggcctgaaagccaagccaaaactgaaaagggctgccccaaccagcaccaggacctcctctgccaagaggaagaaggtgaagaagtaaa

SEQ ID NO: 122 - DNA, 1531 bp, synthetic construct
  misc_feature 1..1531: note = 5205 Synthetic HPV52 L1 gene
  source 1..1531: mol_type = other DNA
ctgggtaccatgagcgtgtggaggcccagcgaggccaccgtgtacctgccccccgtgcccgtgagcaaggtggtgagcaccgacgagtacgtgagcaggaccagcatctactactacgccggcagcagcaggctgctgaccgtgggccacccctacttcagcatcaagaacaccagcagcggcaacggcaagaaggtgctggtgcccaaggtgagcggcctgcagtacagggtgttcaggatcaagctgcccgaccccaacaagttcggcttccccgacaccagcttctacaaccccgagacccagaggctggtgtgggcctgcaccggcctggagatcggcaggggccagcccctgggcgtgggcatcagcggccaccccctgctgaacaagttcgacgacaccgagaccagcaacaagtacgccggcaagcccggcatcgacaacagggagtgcctgagcatggactacaagcagacccagctgtgcatcctgggctgcaagccccccatcggcgagcactggggcaagggcaccccctgcaacaacaacagcggcaaccccggcgactgcccccccctgcagctgatcaacagcgtgatccaggacggcgacatggtggacaccggcttcggctgcatggacttcaacaccctgcaggccagcaagagcgacgtgcccatcgacatctgcagcagcgtgtgcaagtaccccgactacctgcagatggccagcgagccctacggcgacagcctgttcttcttcctgaggagggagcagatgttcgtgaggcacttcttcaacagggccggcaccctgggcgaccccgtgcccggcgacctgtacatccagggcagcaacagcggcaacaccgccaccgtgcagagcagcgccttcttccccacccccagcggcagcatggtgaccagcgagagccagctgttcaacaagccctactggctgcagagggcccagggccacaacaacggcatctgctggggcaaccagctgttcgtgaccgtggtggacaccaccaggagcaccaacatgaccctgtgcgccgaggtgaagaaggagagcacctacaagaacgagaacttcaaggagtacctgaggcacggcgaggagttcgacctgcagttcatcttccagctgtgcaagatcaccctgaccgccgacgtgatgacctacatccacaagatggacgccaccatcctggaggactggcagttcggcctgacccccccccccagcgccagcctggaggacacctacaggttcgtgaccagcaccgccatcacctgccagaagaacaccccccccaagggcaaggaggaccccctgaaggactacatgttctgggaggtggacctgaaggagaagttcagcgccgacctggaccagttccccctgggcaggaagttcctgctgcaggccggcctgcaggccaggcccaagctgaagaggcccgccagcagcgcccccaggaccagcaccaagaagaagaaggtgaagaggtaaactcgagctc

SEQ ID NO: 123 - DNA, 1519 bp, synthetic construct; identical to SEQ ID NO: 6
  misc_feature 1..1519: note = 5206 Synthetic HPV33 L1 gene
  source 1..1519: mol_type = other DNA

SEQ ID NO: 124 - DNA, 34 bp, synthetic construct
  misc_feature 1..34: note = 5207 HPV52L1 F1
  source 1..34: mol_type = other DNA
cttggtaccatgagcgtgtggaggcccagcgagg

SEQ ID NO: 125 - DNA, 35 bp, synthetic construct
  misc_feature 1..35: note = 5208 HPV52L1 R1
  source 1..35: mol_type = other DNA
gcttggctttcaggccggcctgcagcaggaacttc

SEQ ID NO: 126 - DNA, 1453 bp, synthetic construct
  misc_feature 1..1453: note = 5209 HPV52L1 amplified sequence 1
  source 1..1453: mol_type = other DNA
cttggtaccatgagcgtgtggaggcccagcgaggccaccgtgtacctgccccccgtgcccgtgagcaaggtggtgagcaccgacgagtacgtgagcaggaccagcatctactactacgccggcagcagcaggctgctgaccgtgggccacccctacttcagcatcaagaacaccagcagcggcaacggcaagaaggtgctggtgcccaaggtgagcggcctgcagtacagggtgttcaggatcaagctgcccgaccccaacaagttcggcttccccgacaccagcttctacaaccccgagacccagaggctggtgtgggcctgcaccggcctggagatcggcaggggccagcccctgggcgtgggcatcagcggccaccccctgctgaacaagttcgacgacaccgagaccagcaacaagtacgccggcaagcccggcatcgacaacagggagtgcctgagcatggactacaagcagacccagctgtgcatcctgggctgcaagccccccatcggcgagcactggggcaagggcaccccctgcaacaacaacagcggcaaccccggcgactgcccccccctgcagctgatcaacagcgtgatccaggacggcgacatggtggacaccggcttcggctgcatggacttcaacaccctgcaggccagcaagagcgacgtgcccatcgacatctgcagcagcgtgtgcaagtaccccgactacctgcagatggccagcgagccctacggcgacagcctgttcttcttcctgaggagggagcagatgttcgtgaggcacttcttcaacagggccggcaccctgggcgaccccgtgcccggcgacctgtacatccagggcagcaacagcggcaacaccgccaccgtgcagagcagcgccttcttccccacccccagcggcagcatggtgaccagcgagagccagctgttcaacaagccctactggctgcagagggcccagggccacaacaacggcatctgctggggcaaccagctgttcgtgaccgtggtggacaccaccaggagcaccaacatgaccctgtgcgccgaggtgaagaaggagagcacctacaagaacgagaacttcaaggagtacctgaggcacggcgaggagttcgacctgcagttcatcttccagctgtgcaagatcaccctgaccgccgacgtgatgacctacatccacaagatggacgccaccatcctggaggactggcagttcggcctgacccccccccccagcgccagcctggaggacacctacaggttcgtgaccagcaccgccatcacctgccagaagaacaccccccccaagggcaaggaggaccccctgaaggactacatgttctgggaggtggacctgaaggagaagttcagcgccgacctggaccagttccccctgggcaggaagttcctgctgcaggccggcctgaaagccaagc

SEQ ID NO: 127 - DNA, 35 bp, synthetic construct
  misc_feature 1..35: note = 5210 HPV52L1 F2
  source 1..35: mol_type = other DNA
ggccggcctgaaagccaagccaaaactgaaaaggg

SEQ ID NO: 128 - DNA, 36 bp, synthetic construct; identical to SEQ ID NO: 37
  misc_feature 1..36: note = 5211 HPV52L1 R2
  source 1..36: mol_type = other DNA

SEQ ID NO: 129 - DNA, 101 bp, synthetic construct
  misc_feature 1..101: note = 5212 HPV52L1 amplified sequence 2
  source 1..101: mol_type = other DNA
ggccggcctgaaagccaagccaaaactgaaaagggctgccccaaccagcaccaggacctcctctgccaagaggaagaaggtgaagaagtaaatctagacag

SEQ ID NO: 130 - protein, 38 aa, Homo sapiens; identical to SEQ ID NO: 13
  REGION 1..38: note = misc_feature - 5213 Amino acid sequence of HPV type 59 L1 protein aa 471-508

SEQ ID NO: 131 - protein, 467 aa, Homo sapiens
  REGION 1..467: note = misc_feature - 5601 Amino acid sequence of HPV type 56 L1 protein aa 1-467
MATWRPSENKVYLPPTPVSKVVATDSYVKRTSIFYHAGSSRLLAVGHPYYSVTKDNTKTNIPKVSAYQYRVFRVRLPDPNKFGLPDTNIYNPDQERLVWACVGLEVGRGQPLGAGLSGHPLFNRLDDTESSNLANNNVIEDSRDNISVDGKQTQLCIVGCTPAMGEHWTKGAVCKSTQVTTGDCPPLALINTPIEDGDMIDTGFGAMDFKVLQESKAEVPLDIVQSTCKYPDYLKMSADAYGDSMWFYLRREQLFARHYFNRAGKVGETIPAELYLKGSNGREPPPSSVYVATPSGSMITSEAQLFNKPYWLQRAQGHNNGICWGNQLFVTVVDTTRSTNMTISTATEQLSKYDARKINQYLRHVEEYELQFVFQLCKITLSAEVMAYLHNMNANLLEDWNIGLSPPVATSLEDKYRYVRSTAITCQREQPPTEKQDPLAKYKFWDVNLQDSFSTDLDQFPLGRKFL

SEQ ID NO: 132 - protein, 31 aa, Homo sapiens
  REGION 1..31: note = misc_feature - 5602 Amino acid sequence of HPV type 33 L1 protein aa 469-499
LQAGLKAKPKLKRAAPTSTRTSSAKRKKVKK

SEQ ID NO: 133 - protein, 498 aa, synthetic construct
  REGION 1..498: note = 5603 Amino acid sequence of chimeric HPV type 56 L1 protein
MATWRPSENKVYLPPTPVSKVVATDSYVKRTSIFYHAGSSRLLAVGHPYYSVTKDNTKTNIPKVSAYQYRVFRVRLPDPNKFGLPDTNIYNPDQERLVWACVGLEVGRGQPLGAGLSGHPLFNRLDDTESSNLANNNVIEDSRDNISVDGKQTQLCIVGCTPAMGEHWTKGAVCKSTQVTTGDCPPLALINTPIEDGDMIDTGFGAMDFKVLQESKAEVPLDIVQSTCKYPDYLKMSADAYGDSMWFYLRREQLFARHYFNRAGKVGETIPAELYLKGSNGREPPPSSVYVATPSGSMITSEAQLFNKPYWLQRAQGHNNGICWGNQLFVTVVDTTRSTNMTISTATEQLSKYDARKINQYLRHVEEYELQFVFQLCKITLSAEVMAYLHNMNANLLEDWNIGLSPPVATSLEDKYRYVRSTAITCQREQPPTEKQDPLAKYKFWDVNLQDSFSTDLDQFPLGRKFLLQAGLKAKPKLKRAAPTSTRTSSAKRKKVKK

SEQ ID NO: 134 - DNA, 1498 bp, synthetic construct
  misc_feature 1..1498: note = 5604 Nucleotide sequence of chimeric HPV type 56 L1 protein
  source 1..1498: mol_type = other DNA
atggctacctggagaccatctgagaacaaggtctacctgcctccaacacctgtgagcaaggtggtggctacagactcctatgtgaagaggaccagcatcttctaccatgctggctccagcagactgctggctgtgggacacccatactactctgtgaccaaggacaacaccaagaccaacatcccaaaggtgtctgcctaccaatacagggtgttcagggtgagactgcctgacccaaacaagtttggactgcctgacaccaacatctacaaccctgaccaggagagactggtgtgggcttgtgtgggattggaggtgggcaggggacaaccactgggagcaggactgtctggacacccactgttcaacagactggatgacacagagtccagcaacctggctaacaacaatgtgattgaggacagcagggacaacatctctgtggatggcaagcagacccaactttgtattgtgggctgtactcctgctatgggagaacactggaccaagggagcagtgtgtaagagcacccaggtgaccacaggagactgtcctccactggctctgataaacacaccaattgaggatggagatatgattgacacaggctttggagctatggacttcaaggtgctccaagagagcaaggctgaggtgccactggacattgtccagagcacttgtaaataccctgactacctgaaaatgagtgctgatgcctatggagacagtatgtggttctacctgaggagggaacaactttttgccagacactacttcaacagggctggcaaggtgggagagaccatccctgctgaactctacctgaaaggcagcaatggcagggaacctcctccatcctctgtctatgtggctacaccatctggcagtatgattacctctgaggctcaacttttcaacaagccatactggctccaaagggctcaaggacacaacaatggcatctgttggggcaaccaactttttgtgacagtggtggacaccaccaggagcaccaatatgaccatcagcacagccacagaacaacttagcaaatatgatgccaggaagataaaccaatacctgaggcatgtggaggaatatgaactccaatttgtgttccaactttgtaagattaccctgtctgctgaggtgatggcttacctgcacaatatgaatgccaacctgttggaggactggaacattggactgagccctcctgtggctacctccttggaggacaaatacagatatgtgaggagcacagccatcacttgtcagagggaacaacctccaacagagaagcaggacccactggctaaatacaagttctgggatgtgaacctccaagactccttcagcacagacctggaccagtttccactgggcaggaagttcctgctccaagcaggactgaaagccaagccaaaactgaaaagggctgccccaaccagcaccaggacctcctctgccaagaggaagaaggtgaagaagtaaa

SEQ ID NO: 135 - DNA, 1519 bp, synthetic construct
  misc_feature 1..1519: note = 5605 Synthetic HPV56 L1 gene
  source 1..1519: mol_type = other DNA
ctgggtaccatggctacctggagaccatctgagaacaaggtctacctgcctccaacacctgtgagcaaggtggtggctacagactcctatgtgaagaggaccagcatcttctaccatgctggctccagcagactgctggctgtgggacacccatactactctgtgaccaaggacaacaccaagaccaacatcccaaaggtgtctgcctaccaatacagggtgttcagggtgagactgcctgacccaaacaagtttggactgcctgacaccaacatctacaaccctgaccaggagagactggtgtgggcttgtgtgggattggaggtgggcaggggacaaccactgggagcaggactgtctggacacccactgttcaacagactggatgacacagagtccagcaacctggctaacaacaatgtgattgaggacagcagggacaacatctctgtggatggcaagcagacccaactttgtattgtgggctgtactcctgctatgggagaacactggaccaagggagcagtgtgtaagagcacccaggtgaccacaggagactgtcctccactggctctgataaacacaccaattgaggatggagatatgattgacacaggctttggagctatggacttcaaggtgctccaagagagcaaggctgaggtgccactggacattgtccagagcacttgtaaataccctgactacctgaaaatgagtgctgatgcctatggagacagtatgtggttctacctgaggagggaacaactttttgccagacactacttcaacagggctggcaaggtgggagagaccatccctgctgaactctacctgaaaggcagcaatggcagggaacctcctccatcctctgtctatgtggctacaccatctggcagtatgattacctctgaggctcaacttttcaacaagccatactggctccaaagggctcaaggacacaacaatggcatctgttggggcaaccaactttttgtgacagtggtggacaccaccaggagcaccaatatgaccatcagcacagccacagaacaacttagcaaatatgatgccaggaagataaaccaatacctgaggcatgtggaggaatatgaactccaatttgtgttccaactttgtaagattaccctgtctgctgaggtgatggcttacctgcacaatatgaatgccaacctgttggaggactggaacattggactgagccctcctgtggctacctccttggaggacaaatacagatatgtgaggagcacagccatcacttgtcagagggaacaacctccaacagagaagcaggacccactggctaaatacaagttctgggatgtgaacctccaagactccttcagcacagacctggaccagtttccactgggcaggaagttcctgatgcaacttggcaccaggagcaagcctgctgtggctaccagcaagaagaggtctgccccaaccagcaccagcacacctgccaagaggaagaggaggtaaactcgagctc

SEQ ID NO: 136 - DNA, 1519 bp, synthetic construct; identical to SEQ ID NO: 6
  misc_feature 1..1519: note = 5606 Synthetic HPV33 L1 gene
  source 1..1519: mol_type = other DNA

SEQ ID NO: 137 - DNA, 33 bp, synthetic construct
  misc_feature 1..33: note = 5607 HPV56L1 F1
  source 1..33: mol_type = other DNA
cttggtaccatggctacctggagaccatctgag

SEQ ID NO: 138 - DNA, 31 bp, synthetic construct
  misc_feature 1..31: note = 5608 HPV56L1 R1
  source 1..31: mol_type = other DNA
ctgcttggagcaggaacttcctgcccagtgg

SEQ ID NO: 139 - DNA, 1420 bp, synthetic construct
  misc_feature 1..1420: note = 5609 HPV56L1 amplified sequence 1
  source 1..1420: mol_type = other DNA
cttggtaccatggctacctggagaccatctgagaacaaggtctacctgcctccaacacctgtgagcaaggtggtggctacagactcctatgtgaagaggaccagcatcttctaccatgctggctccagcagactgctggctgtgggacacccatactactctgtgaccaaggacaacaccaagaccaacatcccaaaggtgtctgcctaccaatacagggtgttcagggtgagactgcctgacccaaacaagtttggactgcctgacaccaacatctacaaccctgaccaggagagactggtgtgggcttgtgtgggattggaggtgggcaggggacaaccactgggagcaggactgtctggacacccactgttcaacagactggatgacacagagtccagcaacctggctaacaacaatgtgattgaggacagcagggacaacatctctgtggatggcaagcagacccaactttgtattgtgggctgtactcctgctatgggagaacactggaccaagggagcagtgtgtaagagcacccaggtgaccacaggagactgtcctccactggctctgataaacacaccaattgaggatggagatatgattgacacaggctttggagctatggacttcaaggtgctccaagagagcaaggctgaggtgccactggacattgtccagagcacttgtaaataccctgactacctgaaaatgagtgctgatgcctatggagacagtatgtggttctacctgaggagggaacaactttttgccagacactacttcaacagggctggcaaggtgggagagaccatccctgctgaactctacctgaaaggcagcaatggcagggaacctcctccatcctctgtctatgtggctacaccatctggcagtatgattacctctgaggctcaacttttcaacaagccatactggctccaaagggctcaaggacacaacaatggcatctgttggggcaaccaactttttgtgacagtggtggacaccaccaggagcaccaatatgaccatcagcacagccacagaacaacttagcaaatatgatgccaggaagataaaccaatacctgaggcatgtggaggaatatgaactccaatttgtgttccaactttgtaagattaccctgtctgctgaggtgatggcttacctgcacaatatgaatgccaacctgttggaggactggaacattggactgagccctcctgtggctacctccttggaggacaaatacagatatgtgaggagcacagccatcacttgtcagagggaacaacctccaacagagaagcaggacccactggctaaatacaagttctgggatgtgaacctccaagactccttcagcacagacctggaccagtttccactgggcaggaagttcctgctccaagcag

SEQ ID NO: 140 - DNA, 36 bp, synthetic construct
  misc_feature 1..36: note = 5610 HPV56L1 F2
  source 1..36: mol_type = other DNA
gaagttcctgctccaagcaggactgaaagccaagcc

SEQ ID NO: 141 - DNA, 36 bp, synthetic construct; identical to SEQ ID NO: 37
  misc_feature 1..36: note = 5611 HPV56L1 R2
  source 1..36: mol_type = other DNA

SEQ ID NO: 142 - DNA, 116 bp, synthetic construct
  misc_feature 1..116: note = 5612 HPV56L1 amplified sequence 2
  source 1..116: mol_type = other DNA
gaagttcctgctccaagcaggactgaaagccaagccaaaactgaaaagggctgccccaaccagcaccaggacctcctctgccaagaggaagaaggtgaagaagtaaatctagacag

SEQ ID NO: 143 - protein, 38 aa, Homo sapiens; identical to SEQ ID NO: 13
  REGION 1..38: note = misc_feature - 5613 Amino acid sequence of HPV type 59 L1 protein aa 471-508

SEQ ID NO: 144 - protein, 473 aa, Homo sapiens
  REGION 1..473: note = misc_feature - 5801 Amino acid sequence of HPV type 58 L1 protein aa 1-473
MSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKTAPPKEKEDPLNKYTFWEVNLKEKFSADLDQFPLGRKFLLQSGL

SEQ ID NO: 145 - protein, 26 aa, Homo sapiens
  REGION 1..26: note = misc_feature - 5802 Amino acid sequence of HPV type 33 L1 protein aa 474-499
KAKPKLKRAAPTSTRTSSAKRKKVKK

SEQ ID NO: 146 - protein, 499 aa, synthetic construct
  REGION 1..499: note = 5803 Amino acid sequence of chimeric HPV type 58 L1 protein
MSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKTAPPKEKEDPLNKYTFWEVNLKEKFSADLDQFPLGRKFLLQSGLKAKPKLKRAAPTSTRTSSAKRKKVKK

SEQ ID NO: 147 - DNA, 1501 bp, synthetic construct
  misc_feature 1..1501: note = 5804 Nucleotide sequence of chimeric HPV type 58 L1 protein
  source 1..1501: mol_type = other DNA
atgagcgtgtggaggcccagcgaggccaccgtgtacctgccccccgtgcccgtgagcaaggtggtgagcaccgacgagtacgtgagcaggaccagcatctactactacgccggcagcagcaggctgctggccgtgggcaacccctacttcagcatcaagagccccaacaacaacaagaaggtgctggtgcccaaggtgagcggcctgcagtacagggtgttcagggtgaggctgcccgaccccaacaagttcggcttccccgacaccagcttctacaaccccgacacccagaggctggtgtgggcctgcgtgggcctggagatcggcaggggccagcccctgggcgtgggcgtgagcggccacccctacctgaacaagttcgacgacaccgagaccagcaacaggtaccccgcccagcccggcagcgacaacagggagtgcctgagcatggactacaagcagacccagctgtgcctgatcggctgcaagccccccaccggcgagcactggggcaagggcgtggcctgcaacaacaacgccgccgccaccgactgcccccccctggagctgttcaacagcatcatcgaggacggcgacatggtggacaccggcttcggctgcatggacttcggcaccctgcaggccaacaagagcgacgtgcccatcgacatctgcaacagcacctgcaagtaccccgactacctgaagatggccagcgagccctacggcgacagcctgttcttcttcctgaggagggagcagatgttcgtgaggcacttcttcaacagggccggcaagctgggcgaggccgtgcccgacgacctgtacatcaagggcagcggcaacaccgccgtgatccagagcagcgccttcttccccacccccagcggcagcatcgtgaccagcgagagccagctgttcaacaagccctactggctgcagagggcccagggccacaacaacggcatctgctggggcaaccagctgttcgtgaccgtggtggacaccaccaggagcaccaacatgaccctgtgcaccgaggtgaccaaggagggcacctacaagaacgacaacttcaaggagtacgtgaggcacgtggaggagtacgacctgcagttcgtgttccagctgtgcaagatcaccctgaccgccgagatcatgacctacatccacaccatggacagcaacatcctggaggactggcagttcggcctgacccccccccccagcgccagcctgcaggacacctacaggttcgtgaccagccaggccatcacctgccagaagaccgccccccccaaggagaaggaggaccccctgaacaagtacaccttctgggaggtgaacctgaaggagaagttcagcgccgacctggaccagttccccctgggcaggaagttcctgctgcagagcggcctgaaagccaagccaaaactgaaaagggctgccccaaccagcaccaggacctcctctgccaagaggaagaaggtgaagaagtaaa

SEQ ID NO: 148 - DNA, 1516 bp, synthetic construct
  misc_feature 1..1516: note = 5805 Synthetic HPV58 L1 gene
  source 1..1516: mol_type = other DNA
ctgggtaccatgagcgtgtggaggcccagcgaggccaccgtgtacctgccccccgtgcccgtgagcaaggtggtgagcaccgacgagtacgtgagcaggaccagcatctactactacgccggcagcagcaggctgctggccgtgggcaacccctacttcagcatcaagagccccaacaacaacaagaaggtgctggtgcccaaggtgagcggcctgcagtacagggtgttcagggtgaggctgcccgaccccaacaagttcggcttccccgacaccagcttctacaaccccgacacccagaggctggtgtgggcctgcgtgggcctggagatcggcaggggccagcccctgggcgtgggcgtgagcggccacccctacctgaacaagttcgacgacaccgagaccagcaacaggtaccccgcccagcccggcagcgacaacagggagtgcctgagcatggactacaagcagacccagctgtgcctgatcggctgcaagccccccaccggcgagcactggggcaagggcgtggcctgcaacaacaacgccgccgccaccgactgcccccccctggagctgttcaacagcatcatcgaggacggcgacatggtggacaccggcttcggctgcatggacttcggcaccctgcaggccaacaagagcgacgtgcccatcgacatctgcaacagcacctgcaagtaccccgactacctgaagatggccagcgagccctacggcgacagcctgttcttcttcctgaggagggagcagatgttcgtgaggcacttcttcaacagggccggcaagctgggcgaggccgtgcccgacgacctgtacatcaagggcagcggcaacaccgccgtgatccagagcagcgccttcttccccacccccagcggcagcatcgtgaccagcgagagccagctgttcaacaagccctactggctgcagagggcccagggccacaacaacggcatctgctggggcaaccagctgttcgtgaccgtggtggacaccaccaggagcaccaacatgaccctgtgcaccgaggtgaccaaggagggcacctacaagaacgacaacttcaaggagtacgtgaggcacgtggaggagtacgacctgcagttcgtgttccagctgtgcaagatcaccctgaccgccgagatcatgacctacatccacaccatggacagcaacatcctggaggactggcagttcggcctgacccccccccccagcgccagcctgcaggacacctacaggttcgtgaccagccaggccatcacctgccagaagaccgccccccccaaggagaaggaggaccccctgaacaagtacaccttctgggaggtgaacctgaaggagaagttcagcgccgacctggaccagttccccctgggcaggaagttcctgctgcagagcggcctgaaggccaagcccaggctgaagaggagcgcccccaccaccagggcccccagcaccaagaggaagaaggtgaagaagtaaactcgagctc

SEQ ID NO: 149 - DNA, 1519 bp, synthetic construct; identical to SEQ ID NO: 6
  misc_feature 1..1519: note = 5806 Synthetic HPV33 L1 gene
  source 1..1519: mol_type = other DNA

SEQ ID NO: 150 - DNA, 34 bp, synthetic construct; identical to SEQ ID NO: 124
  misc_feature 1..34: note = 5807 HPV58L1 F1
  source 1..34: mol_type = other DNA

SEQ ID NO: 151 - DNA, 36 bp, synthetic construct
  misc_feature 1..36: note = 5808 HPV58L1 R1
  source 1..36: mol_type = other DNA
gcttggctttcaggccgctctgcagcaggaacttcc

SEQ ID NO: 152 - DNA, 1438 bp, synthetic construct
  misc_feature 1..1438: note = 5809 HPV58L1 amplified sequence 1
  source 1..1438: mol_type = other DNA
cttggtaccatgagcgtgtggaggcccagcgaggccaccgtgtacctgccccccgtgcccgtgagcaaggtggtgagcaccgacgagtacgtgagcaggaccagcatctactactacgccggcagcagcaggctgctggccgtgggcaacccctacttcagcatcaagagccccaacaacaacaagaaggtgctggtgcccaaggtgagcggcctgcagtacagggtgttcagggtgaggctgcccgaccccaacaagttcggcttccccgacaccagcttctacaaccccgacacccagaggctggtgtgggcctgcgtgggcctggagatcggcaggggccagcccctgggcgtgggcgtgagcggccacccctacctgaacaagttcgacgacaccgagaccagcaacaggtaccccgcccagcccggcagcgacaacagggagtgcctgagcatggactacaagcagacccagctgtgcctgatcggctgcaagccccccaccggcgagcactggggcaagggcgtggcctgcaacaacaacgccgccgccaccgactgcccccccctggagctgttcaacagcatcatcgaggacggcgacatggtggacaccggcttcggctgcatggacttcggcaccctgcaggccaacaagagcgacgtgcccatcgacatctgcaacagcacctgcaagtaccccgactacctgaagatggccagcgagccctacggcgacagcctgttcttcttcctgaggagggagcagatgttcgtgaggcacttcttcaacagggccggcaagctgggcgaggccgtgcccgacgacctgtacatcaagggcagcggcaacaccgccgtgatccagagcagcgccttcttccccacccccagcggcagcatcgtgaccagcgagagccagctgttcaacaagccctactggctgcagagggcccagggccacaacaacggcatctgctggggcaaccagctgttcgtgaccgtggtggacaccaccaggagcaccaacatgaccctgtgcaccgaggtgaccaaggagggcacctacaagaacgacaacttcaaggagtacgtgaggcacgtggaggagtacgacctgcagttcgtgttccagctgtgcaagatcaccctgaccgccgagatcatgacctacatccacaccatggacagcaacatcctggaggactggcagttcggcctgacccccccccccagcgccagcctgcaggacacctacaggttcgtgaccagccaggccatcacctgccagaagaccgccccccccaaggagaaggaggaccccctgaacaagtacaccttctgggaggtgaacctgaaggagaagttcagcgccgacctggaccagttccccctgggcaggaagttcctgctgcagagcggcctgaaagccaagc

SEQ ID NO: 153 - DNA, 35 bp, synthetic construct
  misc_feature 1..35: note = 5810 HPV58L1 F2
  source 1..35: mol_type = other DNA
gagcggcctgaaagccaagccaaaactgaaaaggg

SEQ ID NO: 154 - DNA, 36 bp, synthetic construct; identical to SEQ ID NO: 37
  misc_feature 1..36: note = 5811 HPV58L1 R2
  source 1..36: mol_type = other DNA

SEQ ID NO: 155 - DNA, 101 bp, synthetic construct
  misc_feature 1..101: note = 5812 HPV58L1 amplified sequence 2
  source 1..101: mol_type = other DNA
gagcggcctgaaagccaagccaaaactgaaaagggctgccccaaccagcaccaggacctcctctgccaagaggaagaaggtgaagaagtaaatctagacag

SEQ ID NO: 156 - protein, 38 aa, Homo sapiens; identical to SEQ ID NO: 13
  REGION 1..38: note = misc_feature - 5813 Amino acid sequence of HPV type 59 L1 protein aa 471-508

SEQ ID NO: 157 - protein, 4 aa, Human Papillomavirus
  REGION 1..4: note = misc_feature - Tetrapeptide in L1 protein of each HPV type
RKFL

SEQ ID NO: 158 - protein, 6 aa, Human Papillomavirus
  REGION 1..6: note = misc_feature - Hexapeptide in L1 protein of each HPV type
LGRKFL

SEQ ID NO: 159 - protein, 18 aa, Human Papillomavirus
  REGION 1..18: note = misc_feature - Epitope of the L1 protein
EEYDLQFIFQLCKITLTA

SEQ ID NO: 160 - protein, 21 aa, Human Papillomavirus
  REGION 1..21: note = misc_feature - Epitope of the L1 protein
RHGEEYDLQFIFQLCKITLTA

SEQ ID NO: 161 - protein, 7 aa, Human Papillomavirus
  REGION 1..7: note = misc_feature - Epitope of the L1 protein
LPDPNKF

SEQ ID NO: 162 - protein, 10 aa, Human Papillomavirus
  REGION 1..10: note = misc_feature - Epitope of the L1 protein
PETQRLVWAC

SEQ ID NO: 163 - protein, 8 aa, Human Papillomavirus
  REGION 1..8: note = misc_feature - Epitope of the L1 protein
PVPGQYDA

SEQ ID NO: 164 - protein, 12 aa, Human Papillomavirus
  REGION 1..12: note = misc_feature - Epitope of the L1 protein
YNPETQRLVWAC

SEQ ID NO: 165 - protein, 8 aa, Human Papillomavirus
  REGION 1..8: note = misc_feature - Epitope of the L1 protein
DTGYGAMD

SEQ ID NO: 166 - protein, 10 aa, Human Papillomavirus
  REGION 1..10: note = misc_feature - Epitope of the L1 protein
PVPGQYDATK

SEQ ID NO: 167 - protein, 17 aa, Human Papillomavirus
  REGION 1..17: note = misc_feature - Epitope of the L1 protein
KQDIPKVSAYQYRVFRV

SEQ ID NO: 168 - protein, 15 aa, Human Papillomavirus
  REGION 1..15: note = misc_feature - Epitope of the L1 protein
RDNVSVDYKQTQLCI

SEQ ID NO: 169 - protein, 14 aa, Human Papillomavirus
  REGION 1..14: note = misc_feature - Epitope of the L1 protein
YSRHVEEYDLQFIF

SEQ ID NO: 170 - protein, 505 aa, Human Papillomavirus
  REGION 1..505: note = misc_feature - Amino Acid Sequence of Native HPV16L1
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 171 - protein, 502 aa, Human Papillomavirus
  REGION 1..502: note = misc_feature - Amino Acid Sequence of Native HPV35L1
MSLWRSNEATVYLPPVSVSKVVSTDEYVTRTNIYYHAGSSRLLAVGHPYYAIKKQDSNKIAVPKVSGLQYRVFRVKLPDPNKFGFPDTSFYDPASQRLVWACTGVEVGRGQPLGVGISGHPLLNKLDDTENSNKYVGNSGTDNRECISMDYKQTQLCLIGCRPPIGEHWGKGTPCNANQVKAGECPPLELLNTVLQDGDMVDTGFGAMDFTTLQANKSDVPLDICSSICKYPDYLKMVSEPYGDMLFFYLRREQMFVRHLFNRAGTVGETVPADLYIKGTTGTLPSTSYFPTPSGSMVTSDAQIFNKPYWLQRAQGHNNGICWSNQLFVTVVDTTRSTNMSVCSAVSSSDSTYKNDNFKEYLRHGEEYDLQFIFQLCKITLTADVMTYIHSMNPSILEDWNFGLTPPPSGTLEDTYRYVTSQAVTCQKPSAPKPKDDPLKNYTFWEVDLKEKFSADLDQFPLGRKFLLQAGLKARPNFRLGKRAAPASTSKKSSTKRRKVKS

SEQ ID NO: 172 - protein, 501 aa, Human Papillomavirus
  REGION 1..501: note = misc_feature - Amino Acid Sequence of Native HPV11L1
MWRPSDSTVYVPPPNPVSKVVATDAYVKRTNIFYHASSSRLLAVGHPYYSIKKVNKTVVPKVSGYQYRVFKVVLPDPNKFALPDSSLFDPTTQRLVWACTGLEVGRGQPLGVGVSGHPLLNKYDDVENSGGYGGNPGQDNRVNVGMDYKQTQLCMVGCAPPLGEHWGKGTQCSNTSVQNGDCPPLELITSVIQDGDMVDTGFGAMNFADLQTNKSDVPLDICGTVCKYPDYLQMAADPYGDRLFFYLRKEQMFARHFFNRAGTVGEPVPDDLLVKGGNNRSSVASSIYVHTPSGSLVSSEAQLFNKPYWLQKAQGHNNGICWGNHLFVTVVDTTRSTNMTLCASVSKSATYTNSDYKEYMRHVEEFDLQFIFQLCSITLSAEVMAYIHTMNPSVLEDWNFGLSPPPNGTLEDTYRYVQSQAITCQKPTPEKEKQDPYKDMSFWEVNLKEKFSSELDQFPLGRKFLLQSGYRGRTSARTGIKRPAVSKPSTAPKRKRTKTKK

SEQ ID NO: 173 - protein, 507 aa, Human Papillomavirus
  REGION 1..507: note = misc_feature - Amino Acid Sequence of Native HPV18L1
MALWRPSDNTVYLPPPSVARVVNTDDYVTRTSIFYHAGSSRLLTVGNPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKRVRVRARK

SEQ ID NO: 174 - protein, 500 aa, Human Papillomavirus
  REGION 1..500: note = misc_feature - Amino Acid Sequence of Native HPV6L1
MWRPSDSTVYVPPPNPVSKVVATDAYVTRTNIFYHASSSRLLAVGHPYFSIKRANKTVVPKVSGYQYRVFKVVLPDPNKFALPDSSLFDPTTQRLVWACTGLEVGRGQPLGVGVSGHPFLNKYDDVENSGSGGNPGQDNRVNVGMDYKQTQLCMVGCAPPLGEHWGKGKQCTNTPVQAGDCPPLELITSVIQDGDMVDTGFGAMNFADLQTNKSDVPIDICGTTCKYPDYLQMAADPYGDRLFFFLRKEQMFARHFFNRAGEVGEPVPDTLIIKGSGNRTSVGSSIYVNTPSGSLVSSEAQLFNKPYWLQKAQGHNNGICWGNQLFVTVVDTTRSTNMTLCASVTTSSTYTNSDYKEYMRHVEEYDLQFIFQLCSITLSAEVMAYIHTMNPSVLEDWNFGLSPPPNGTLEDTYRYVQSQAITCQKPTPEKEKPDPYKNLSFWEVNLKEKFSSELDQYPLGRKFLLQSGYRGRSSIRTGVKRPAVSKASAAPKRKRAKTKR

SEQ ID NO: 175 - protein, 503 aa, Human Papillomavirus
  REGION 1..503: note = misc_feature - Amino Acid Sequence of Native HPV52L1
MSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKDYMFWEVDLKEKFSADLDQFPLGRKFLLQAGLQARPKLKRPASSAPRTSTKKKKVKR

SEQ ID NO: 176 - protein, 498 aa, Human Papillomavirus
  REGION 1..498: note = misc_feature - Amino Acid Sequence of Native HPV58L1
MSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKTAPPKEKEDPLNKYTFWEVNLKEKFSADLDQFPLGRKFLLQSGLKAKPRLKRSAPTTRAPSTKRKKVKK

SEQ ID NO: 177 - protein, 499 aa, Human Papillomavirus
  REGION 1..499: note = misc_feature - Amino Acid Sequence of Native HPV33L1
MSVWRPSEATVYLPPVPVSKVVSTDEYVSRTSIYYYAGSSRLLAVGHPYFSIKNPTNAKKLLVPKVSGLQYRVFRVRLPDPNKFGFPDTSFYNPDTQRLVWACVGLEIGRGQPLGVGISGHPLLNKFDDTETGNKYPGQPGADNRECLSMDYKQTQLCLLGCKPPTGEHWGKGVACTNAAPANDCPPLELINTIIEDGDMVDTGFGCMDFKTLQANKSDVPIDICGSTCKYPDYLKMTSEPYGDSLFFFLRREQMFVRHFFNRAGTLGEAVPDDLYIKGSGTTASIQSSAFFPTPSGSMVTSESQLFNKPYWLQRAQGHNNGICWGNQVFVTVVDTTRSTNMTLCTQVTSDSTYKNENFKEYIRHVEEYDLQFVFQLCKVTLTAEVMTYIHAMNPDILEDWQFGLTPPPSASLQDTYRFVTSQAITCQKTVPPKEKEDPLGKYTFWEVDLKEKFSADLDQFPLGRKFLLQAGLKAKPKLKRAAPTSTRTSSAKRKKVKK

SEQ ID NO: 178 - protein, 504 aa, Human Papillomavirus
  REGION 1..504: note = misc_feature - Amino Acid Sequence of Native HPV31L1
MSLWRPSEATVYLPPVPVSKVVSTDEYVTRTNIYYHAGSARLLTVGHPYYSIPKSDNPKKIVVPKVSGLQYRVFRVRLPDPNKFGFPDTSFYNPETQRLVWACVGLEVGRGQPLGVGISGHPLLNKFDDTENSNRYAGGPGTDNRECISMDYKQTQLCLLGCKPPIGEHWGKGSPCSNNAITPGDCPPLELKNSVIQDGDMVDTGFGAMDFTALQDTKSNVPLDICNSICKYPDYLKMVAEPYGDTLFFYLRREQMFVRHFFNRSGTVGESVPTDLYIKGSGSTATLANSTYFPTPSGSMVTSDAQIFNKPYWMQRAQGHNNGICWGNQLFVTVVDTTRSTNMSVCAAIANSDTTFKSSNFKEYLRHGEEFDLQFIFQLCKITLSADIMTYIHSMNPAILEDWNFGLTTPPSGSLEDTYRFVTSQAITCQKSAPQKPKEDPFKDYVFWEVNLKEKFSADLDQFPLGRKFLLQAGYRARPKFKAGKRSAPSASTTTPAKRKKTKK

SEQ ID NO: 179 - protein, 504 aa, Human Papillomavirus
  REGION 1..504: note = misc_feature - Amino Acid Sequence of Native HPV51L1
MALWRTNDSKVYLPPAPVSRIVNTEEYITRTGIYYYAGSSRLITLGHPYFPIPKTSTRAAIPKVSAFQYRVFRVQLPDPNKFGLPDPNLYNPDTDRLVWGCVGVEVGRGQPLGVGLSGHPLFNKYDDTENSRIANGNAQQDVRDNTSVDNKQTQLCIIGCAPPIGEHWGIGTTCKNTPVPPGDCPPLELVSSVIQDGDMIDTGFGAMDFAALQATKSDVPLDISQSVCKYPDYLKMSADTYGNSMFFHLRREQIFARHYYNKLVGVGEDIPNDYYIKGSGNGRDPIESYIYSATPSGSMITSDSQIFNKPYWLHRAQGHNNGICWNNQLFITCVDTTRSTNLTISTATAAVSPTFTPSNFKQYIRHGEEYELQFIFQLCKITLTTEVMAYLHTMDPTILEQWNFGLTLPPSASLEDAYRFVRNAATSCQKDTPPQAKPDPLAKYKFWDVDLKERFSLDLDQFALGRKFLLQVGVQRKPRPGLKRPASSASSSSSSSAKRKRVKK

SEQ ID NO: 180 - protein, 499 aa, Human Papillomavirus
  REGION 1..499: note = misc_feature - Amino Acid Sequence of Native HPV56L1
MATWRPSENKVYLPPTPVSKVVATDSYVKRTSIFYHAGSSRLLAVGHPYYSVTKDNTKTNIPKVSAYQYRVFRVRLPDPNKFGLPDTNIYNPDQERLVWACVGLEVGRGQPLGAGLSGHPLFNRLDDTESSNLANNNVIEDSRDNISVDGKQTQLCIVGCTPAMGEHWTKGAVCKSTQVTTGDCPPLALINTPIEDGDMIDTGFGAMDFKVLQESKAEVPLDIVQSTCKYPDYLKMSADAYGDSMWFYLRREQLFARHYFNRAGKVGETIPAELYLKGSNGREPPPSSVYVATPSGSMITSEAQLFNKPYWLQRAQGHNNGICWGNQLFVTVVDTTRSTNMTISTATEQLSKYDARKINQYLRHVEEYELQFVFQLCKITLSAEVMAYLHNMNANLLEDWNIGLSPPVATSLEDKYRYVRSTAITCQREQPPTEKQDPLAKYKFWDVNLQDSFSTDLDQFPLGRKFLMQLGTRSKPAVATSKKRSAPTSTSTPAKRKRR

SEQ ID NO: 181 - protein, 505 aa, Human Papillomavirus
  REGION 1..505: note = misc_feature - Amino Acid Sequence of Native HPV39L1
MAMWRSSDSMVYLPPPSVAKVVNTDDYVTRTGIYYYAGSSRLLTVGHPYFKVGMNGGRKQDIPKVSAYQYRVFRVTLPDPNKFSIPDASLYNPETQRLVWACVGVEVGRGQPLGVGISGHPLYNRQDDTENSPFSSTTNKDSRDNVSVDYKQTQLCIIGCVPAIGEHWGKGKACKPNNVSTGDCPPLELVNTPIEDGDMIDTGYGAMDFGALQETKSEVPLDICQSICKYPDYLQMSADVYGDSMFFCLRREQLFARHFWNRGGMVGDAIPAQLYIKGTDIRANPGSSVYCPSPSGSMVTSDSQLFNKPYWLHKAQGHNNGICWHNQLFLTVVDTTRSTNFTLSTSIESSIPSTYDPSKFKEYTRHVEEYDLQFIFQLCTVTLTTDVMSYIHTMNSSILDNWNFAVAPPPSASLVDTYRYLQSAAITCQKDAPAPEKKDPYDGLKFWNVDLREKFSLELDQFPLGRKFLLQARVRRRPTIGPRKRPAASTSSSSATKHKRKRVSK

SEQ ID NO: 182 - protein, 508 aa, Human Papillomavirus
  REGION 1..508: note = misc_feature - Amino Acid Sequence of Native HPV59L1
MALWRSSDNKVYLPPPSVAKVVSTDEYVTRTSIFYHAGSSRLLTVGHPYFKVPKGGNGRQDVPKVSAYQYRVFRVKLPDPNKFGLPDNTVYDPNSQRLVWACVGVEIGRGQPLGVGLSGHPLYNKLDDTENSHVASAVDTKDTRDNVSVDYKQTQLCIIGCVPAIGEHWTKGTACKPTTVVQGDCPPLELINTPIEDGDMVDTGYGAMDFKLLQDNKSEVPLDICQSICKYPDYLQMSADAYGDSMFFCLRREQVFARHFWNRSGTMGDQLPESLYIKGTDIRANPGSYLYSPSPSGSVVTSDSQLFNKPYWLHKAQGLNNGICWHNQLFLTVVDTTRSTNLSVCASTTSSIPNVYTPTSFKEYARHVEEFDLQFIFQLCKITLTTEVMSYIHNMNTTILEDWNFGVTPPPTASLVDTYRFVQSAAVTCQKDTAPPVKQDPYDKLKFWPVDLKERFSADLDQFPLGRKFLLQLGARPKPTIGPRKRAAPAPTSTPSPKRVKRRKSSRK

SEQ ID NO: 183 - protein, 510 aa, Human Papillomavirus
  REGION 1..510: note = misc_feature - Amino Acid Sequence of Native HPV45L1
MALWRPSDSTVYLPPPSVARVVNTDDYVSRTSIFYHAGSSRLLTVGNPYFRVVPSGAGNKQAVPKVSAYQYRVFRVALPDPNKFGLPDSTIYNPETQRLVWACVGMEIGRGQPLGIGLSGHPFYNKLDDTESAHAATAVITQDVRDNVSVDYKQTQLCILGCVPAIGEHWAKGTLCKPAQLQPGDCPPLELKNTIIEDGDMVDTGYGAMDFSTLQDTKCEVPLDICQSICKYPDYLQMSADPYGDSMFFCLRREQLFARHFWNRAGVMGDTVPTDLYIKGTSANMRETPGSCVYSPSPSGSITTSDSQLFNKPYWLHKAQGHNNGICWHNQLFVTVVDTTRSTNLTLCASTQNPVPNTYDPTKFKHYSRHVEEYDLQFIFQLCTITLTAEVMSYIHSMNSSILENWNFGVPPPPTTSLVDTYRFVQSVAVTCQKDTTPPEKQDPYDKLKFWTVDLKEKFSSDLDQYPLGRKFLVQAGLRRRPTIGPRKRPAASTSTASRPAKRVRIRSKK

SEQ ID NO: 184 - protein, 17 aa, Human Papillomavirus
  REGION 1..17: note = misc_feature - HPV6
ELDQYPLGRKFLLQSGY

SEQ ID NO: 185 - protein, 17 aa, Human Papillomavirus
  REGION 1..17: note = misc_feature - HPV11
ELDQFPLGRKFLLQSGY

SEQ ID NO: 186 - protein, 17 aa, Human Papillomavirus
  REGION 1..17: note = misc_feature - HPV16
DLDQFPLGRKFLLQAGL

SEQ ID NO: 187 - protein, 17 aa, Human Papillomavirus
  REGION 1..17: note = misc_feature - HPV18
DLDQYPLGRKFLVQAGL

SEQ ID NO: 188 - protein, 17 aa, Human Papillomavirus
  REGION 1..17: note = misc_feature - HPV31
DLDQFPLGRKFLLQAGY

SEQ ID NO: 189 - protein, 17 aa, Human Papillomavirus
  REGION 1..17: note = misc_feature - HPV35
DLDQFPLGRKFLLQAGL

SEQ ID NO: 190 - protein, 17 aa, Human Papillomavirus
  REGION 1..17: note = misc_feature - HPV39
ELDQFPLGRKFLLQARV

SEQ ID NO: 191 - protein, 17 aa, Human Papillomavirus
  REGION 1..17: note = misc_feature - HPV45
DLDQYPLGRKFLVQAGL

SEQ ID NO: 192 - protein, 17 aa, Human Papillomavirus
  REGION 1..17: note = misc_feature - HPV51
DLDQFALGRKFLLQVGV

SEQ ID NO: 193 - protein, 17 aa, Human Papillomavirus
  REGION 1..17: note = misc_feature - HPV52
DLDQFPLGRKFLLQAGL

SEQ ID NO: 194 - protein, 19 aa, Human Papillomavirus
  REGION 1..19: note = misc_feature - HPV56
DLDQFPLGRKFLMQLGTRS

SEQ ID NO: 195 - protein, 17 aa, Human Papillomavirus
  REGION 1..17: note = misc_feature - HPV58
DLDQFPLGRKFLLQSGL

SEQ ID NO: 196 - protein, 43 aa, Human Papillomavirus
  REGION 1..43: note = misc_feature - HPV33
DLDQFPLGRKFLLQAGLKAKPKLKRAAPTSTRTSSAKRKKVKK

SEQ ID NO: 197 - protein, 4 aa, Human Papillomavirus
  REGION 1..4: note = misc_feature - nuclear localization sequence
KRKK

SEQ ID NO: 198 - protein, 50 aa, Human Papillomavirus
  REGION 1..50: note = misc_feature - HPV59
DLDQFPLGRKFLLQLGARPKPTIGPRKRAAPAPTSTPSPKRVKRRKSSRK

SEQ ID NO: 199 - protein, 7 aa, Human Papillomavirus
  REGION 1..7: note = misc_feature - nuclear localization sequence
KRVKRRK

SEQ ID NO: 200 - protein, 4 aa, Human Papillomavirus
  REGION 1..4: note = misc_feature - nuclear localization sequence
KRKR

SEQ ID NO: 201 - protein, 5 aa, Human Papillomavirus
  REGION 1..5: note = misc_feature - nuclear localization sequence
KRKRK

SEQ ID NO: 202 - protein, 6 aa, Human Papillomavirus
  REGION 1..6: note = misc_feature - nuclear localization sequence
KRKKRK